(19) United States
(12) Patent Application Publication
(10) Pub. No.: US 2005/0276858 A1
(43) Pub. Date: Dec. 15, 2005

(54) Bifunctional-modified hydrogels

(76) Inventors: Weiyuan John Kao, Middleton, WI (US); Jeffrey M. Phillips, Neenah, WI (US); Jing Li, Blaine, MN (US); David Lok, Madison, WI (US); Rathna Gundloori, Maharastra (IN)

Correspondence Address: Joseph T. Leone;DeWitt Rosa & Stevens S.C., 8000 Excelsior Drive, Madison, WI, 53717, US

(21) Appl. No.: 11/093,531

(22) Filed: Mar. 30, 2005

Related U.S. Application Data
(63) Continuation-in-part of application No. 10/128,198, filed on Apr. 23, 2002.
(60) Provisional application No. 60/285,782, filed on Apr. 23, 2001.

(51) Int. Cl. A61K 9/70; A61K 9/14

(52) U.S. Cl. 424/487

(57) ABSTRACT

Disclosed are hydrogels wherein a polymer matrix is modified to contain a bifunctional poly(alkylene glycol) molecule covalently bonded to the polymer matrix. The hydrogels can be cross-linked using, for example, glutaraldehyde. The hydrogels may also be crosslinked via an interpenetrating network of a photopolymerizable acrylates. The hydrogels may also be modified to have pharmacologically-active agents covalently bonded to the poly(alkylene glycol) molecules or entrained within the hydrogel. Living cells may also be entrained within the hydrogels.

### CROSS-REFERENCE TO RELATED APPLICATIONS

This is a continuation-in-part of co-pending application Ser. No. 10/128,198, filed Apr. 23, 2002, which claims priority to provisional application Ser. No. 60/285,782, filed Apr. 23, 2001, both of which are incorporated herein by reference.

### FEDERAL FUNDING

This invention was made with United States government support awarded by the following agencies: NIH EB000290. The United States has certain rights in this invention.

### FIELD OF THE INVENTION

The invention is directed to hydrogels modified using bifunctional reagents, use of the hydrogels to deliver drugs or other biologically-active agents to a mammal in need thereof, compositions containing the hydrogels described herein, and devices, such as wound dressings, diapers, catamenial devices, etc., incorporating the hydrogels.

### INCORPORATION BY REFERENCE

All of the references listed in the “References” section are incorporated herein by reference.

### BACKGROUND

Biological systems, such as healing and embryonic development, operate under spatially- and temporally-controlled orchestration. A myriad of signals and cells all act, in space and time, to heal a cut, for example, or to surround and neutralize a foreign body. The efficacy of current materials used to construct biomedical devices is limited by a lack of multi-functional structures to complement the inherent dynamics of these biological systems.

For example, most wound dressings provide nothing more than a simple barrier to shield the wound and to prevent foreign objects from entering the would. Other newer types of dressings also include antibiotics to prevent sepsis at the wound site. However, these dressings do not address, for example, the exudation which occurs from a wound. Thus, these dressings must be changed often.

Certain biodegradable polymers have been used in burn dressings, hemostatic patches, and the like. These biodegradable polymers provide a barrier and possibly a tissue scaffold for regrowth. However, these types of dressings have no therapeutic effect. While such types of dressings provide effective barriers to physical disturbance of the wound site, scarring is still extensive.

Despite the extensive investigation of novel wound dressing materials, very few materials are in current clinical use. An ideal functional wound dressing should have the following properties: It should be non-toxic, biocompatible, and permeable to moisture and gases to absorb wound exudate and toxins as well to maintain appropriate humidity and oxygen levels. It should be porous to prevent swelling of the wound bed and accumulation of the fluid between the wound site and the material. It should be flexible and durable and minimize local inflammation and infection, thereby promoting new vascularization, re-epithelialization, and normal healing.

Hydrogels are three-dimensional networks capable of absorbing copious amounts of water. Hydrogels have been explored for many uses, including drug delivery devices, wound dressing materials, contact lenses, and cell transplantation matrices. Edible hydrogels, such as gelatin, find extensive use in various food-related applications, such as texture modification, gelling, clarification of beers and wines, and as medicine capsules.

### SUMMARY OF THE INVENTION

The invention is directed to hydrogels comprising a polymer matrix, preferably gelatin or a synthetic polymer (preferably a biodegradable polymer, although the polymer may also be non-biodegradable), modified to contain bifunctional poly(alkylene glycols) covalently bonded to the polymer matrix. Heterobifunctional, poly-C₁-C₆-poly(alkylene glycol) molecules, preferably poly(ethylene glycol) molecules (hPEGs), each having an α-terminus and an ω-terminus, are bonded to the polymer backbone via covalent bonds involving either of the α- or ω-termini. One or more biofunctional agents (i.e., pharmacologically-active agents) are then bonded to the other of the α- or ω-termini (i.e., the free termini) of the hPEGs, thereby yielding a modified, pharmacologically active, homogenous, and covalently-assembled hydrogel. A schematic representation of the preferred embodiment of the invention is shown in FIG. 4.

Any pharmacologically-active agent, without limitation, can be incorporated into the hydrogel, including (by way of illustration and not limitation) vulnerary agents, hemostatic agents, antibiotics, antithelmintics, anti-fungal agents, hormones, anti-inflammatory agents, proteins, polypeptides, oligonucleotides, cytokines, enzymes, etc.

The hydrogels of the present invention find many uses, the preferred of which is as a functional wound dressing. In this preferred embodiment, the hydrogel contains as a pharmacologically-active agent a vulnerary agent covalently bonded to a biodegradable polymer matrix via a differentially-modified, α- and ω-substituted PEG linker.

The hydrogels of the present invention may also be incorporated into bandages, surgical and dental wound packing material, diapers and catamenial devices, and the like.

The novel hydrogel constructs described herein are not physical blends, which are common in the formulation of current biomedical hydrogels; hence, the chemical and physical properties of the subject hydrogels are homogenous and can be tailored to suit a particular clinical end-point requirement. Furthermore, the hydrogel constructs are mechanically stable because the components are covalently bonded. In addition, the hydrophilicity and flexibility of the porous hydrogel accommodate the absorption of wound exudate and assist the final removal of the material from the wound site (if necessary or desired). The nature of gelatin and the porosity of the construct further facilitate the exchange of gases and allow healing. Most importantly, the presence of hPEG-conjugated bioactive compounds and the loading of other pharmaceutical compounds within the matrix allows for the temporally- and spatially-controlled delivery of bioactive signals to modulate and complement the dynamics of the host healing process.

The present invention offers several key commercial advantages over existing products. For example, despite the extensive investigation in the development of novel wound dressing materials, very few materials are used clinically due to the multiple requirements necessary for a functional wound dressing. Ideal functional wound dressings must be nontoxic, biocompatible, permeable to moisture and gases to absorb wound exudate and toxins, as well as to maintain humidity and oxygen levels. The dressings should be porous to prevent swelling of the wound bed and to prevent accumulation of fluid between the wound site and the material. They should be flexible and durable. They should be biocompatible and minimize local inflammation and infection. They should promote neovascularization, re-epithelialization, and normal healing. The novel multi-functional hydrogels described herein can be made to address all of the above requirements for a clinically viable wound dressing material.

Thus in a first embodiment, the invention is directed to a hydrogel that comprises a polymer matrix. The preferred polymer matrix contains reactive amino groups. The most preferred polymer matrices are gelatin and collagen. The polymer matrix is modified using a bifunctional modifier comprising a poly(alkylene glycol) molecule having a substituted or unsubstituted α-terminus and a substituted or unsubstituted ω-terminus. At least one of the α- or ω-termini is covalently bonded to the polymer matrix. The other terminus projects into the interior of the hydrogel mass and modifies its physico-chemical properties. By controlling the nature of the α- and ω-termini, the physical and chemical qualities of the resulting hydrogel can be altered.

Additionally, in the preferred embodiment, the α- and ω-termini are different, and thus are differentially reactive. This enables, for example, one or more pharmacologically-active agents to be covalently bonded to one of the α- or ω-termini that is not bonded to the polymer matrix. Alternatively (or simultaneously), one or more pharmacologically-active agents may also be entrained within the hydrogel.

The polymer matrix of the hydrogel may be cross-linked with a cross-linking reagent such as glutaraldehyde. Cross-linking alters the absorption characteristics and material strength of the resulting gel. Thus, cross-linking may be desirable where increased mechanical strength of the gel is required.

As noted above, the α- and/or ω-termini of the hydrogel may be substituted or unsubstituted. When substituted, it is preferred that the substitution is a moiety selected from the group consisting of halo, hydroxy, C₁-C₂₄-alkyl, C₁-C₂₄-alkenyl, C₁-C₂₄-alkynyl, C₁-C₂₄-alkoxy, C₁-C₂₄-heteroalkyl, C₁-C₂₄-heteroalkenyl, C₁-C₂₄-heteroalkynyl, cyano-C₁-C₂₄-alkyl, C₃-C₁₀-cycloalkyl, C₃-C₁₀-cycloalkenyl, C₃-C₁₀-cycloalkynyl, C₃-C₁₀-cycloheteroalkyl, C₃-C₁₀-cycloheteroalkenyl, C₃-C₁₀-cycloheteroalkynyl, acyl, acyl-C₁-C₂₄-alkyl, acyl-C₁-C₂₄-alkenyl, acyl-C₁-C₂₄-alkynyl, carboxy, C₁-C₂₄-alkylcarboxy, C₁-C₂₄-alkenylcarboxy, C₁-C₂₄-alkynylcarboxy, carboxy-C₁-C₂₄-alkyl, carboxy-C₁-C₂₄-alkenyl, carboxy-C₁-C₂₄-alkynyl, aryl, aryl-C₁-C₂₄-alkyl, aryl-C₁-C₂₄-alkenyl, aryl-C₁-C₂₄-alkynyl, heteroaryl, heteroaryl-C₁-C₂₄-alkyl, heteroaryl-C₁-C₂₄-alkenyl, heteroaryl-C₁-C₂₄-alkynyl, sulfonate, arylsulfonate, and heteroarylsulfonate.

Moreover, these moieties themselves may be further substituted. Thus, the moieties on the α-terminus and the ω-terminus when substituted bear a substituent selected from the group consisting of alkyl, aryl, acyl, halogen, hydroxy, amino, alkoxy, alkylamino, acylamino, thioamido, acyloxy, aryloxy, aryloxyalkyl, mercapto, thia, aza, oxo, saturated cyclic hydrocabon, unsaturated cyclic hydrocarbon, heterocycle, aryl, and heteroaryl.

More specifically, the invention is directed to a hydrogel comprising:

a polymer matrix containing reactive amino acid moieties; and

a bifunctional modifier comprising a compound of formula: 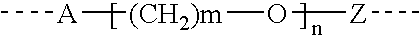
wherein at least one of the “A” or “Z” moieties is covalently bonded to the reactive amino moieties of the polymer matrix; and wherein “A” and “Z” are independently selected from the group consisting of hydrogen, halo, hydroxy, C₁-C₂₄-alkyl, C₁-C₂₄-alkenyl, C₁-C₂₄-alkynyl, C₁-C₂₄-alkoxy, C₁-C₂₄-heteroalkyl, C₁-C₂₄-heteroalkenyl, C₁-C₂₄-heteroalkynyl, cyano-C₁-C₂₄-alkyl, C₃-C₁₀-cycloalkyl, C₃-C₁₀-cycloalkenyl, C₃-C₁₀-cycloalkynyl, C₃-C₁₀-cycloheteroalkyl, C₃-C₁₀-cycloheteroalkenyl, C₃-C₁₀-cycloheteroalkynyl, acyl, acyl-C₁-C₂₄-alkyl, acyl-C₁-C₂₄-alkenyl, acyl-C₁-C₂₄-alkynyl, carboxy, C₁-C₂₄-alkylcarboxy, C₁-C₂₄-alkenylcarboxy, C₁-C₂₄-alkynylcarboxy, carboxy-C₁-C₂₄-alkyl, carboxy-C₁-C₂₄-alkenyl, carboxy-C₁-C₂₄-alkynyl, aryl, aryl-C₁-C₂₄-alkyl, aryl-C₁-C₂₄-alkenyl, aryl-C₁-C₂₄-alkynyl, heteroaryl, heteroaryl-C₁-C₂₄-alkyl, heteroaryl-C₁-C₂₄-alkenyl, heteroaryl-C₁-C₂₄-alkynyl, sulfonate, arylsulfonate, and heteroarylsulfonate; “m” is an integer of from 2 to 8; and n” is an integer equal to or greater than 100. In the preferred embodiment, “m” equals 2 and “n” is greater than 2,000.

A second embodiment of the invention is directed to a hydrogel as described above, with the further inclusion of a second polymer matrix. In this embodiment, the second polymer matrix interpenetrates with the first polymer matrix. Thus, the first polymer matrix, with its grafted modifier molecules, interpenetrates and is physically bound within a second, interpenetrating polymer matrix. In the preferred second embodiment, the second polymer matrix comprises a photopolymerized poly(acrylate), such as an α-acrylate-ω-acrylate-poly(alkylene glycol), trimethylolpropane triacrylate, acrylic acid, and/or acryloyl halide. The second polymer matrix may be a homo-polymer or co-polymer or two or more monomer types.

As in the first embodiment, the interpenetrating hydrogels may further comprise a pharmacologically-active agent covalently bonded to one of the α- or ω-termini that is not bonded to the first polymer matrix.

Likewise, all of the hydrogels according to the present invention may further comprise a pharmacologically-active agent or a living cell entrained within the hydrogel.

A third embodiment of the invention is directed to a method of making a hydrogel as described hereinabove. The method comprises reacting a polymer matrix with a bifunctional modifier comprising a poly(alkylene glycol) molecule having a substituted or unsubstituted α-terminus and a substituted or unsubstituted ω-terminus, whereby at least one of the α- or ω-termini is covalently bonded to the polymer matrix.

A fourth embodiment of the invention is directed to the method described in the previous paragraph, and further comprising contacting the first polymer matrix with a plurality of monomers and then polymerizing the monomers to yield a second polymer matrix, wherein the second polymer matrix interpenetrates with the first polymer matrix. This embodiment allows for the in situ formation of interpenetrating polymer networks

The hydrogels of the present invention can be used in any application where hydrogels are currently employed. Thus, the hydrogels of the present invention find use as wound dressings, diapers, catamenial devices, and the like. In one embodiment, the hydrogels are used to administer a pharmacologically-active agent to a patient in need of the pharmacologically-active agent. In this use, the pharmacologically-active agent either is covalently bonded within the gel or entrained within the gel. The gel is then administered to the patient, as by packing it into a surgical or traumatic wound.

The hydrogels of the present invention are also useful as scaffolds to support living cells. Thus, the hydrogels of the present invention can be used as biomechanical devices. The hydrogels will support living cells within the bulk of the gel, thereby providing a three-dimensional support network in which the cells can grow and proliferate. Hydrogels according to the present invention that contain cells can be implanted into a patient in need of such cells.

### BRIEF DESCRIPTION OF THE DRAWINGS

FIG. 1. A summary of the chemical reactions and structure of critical intermediates and final products of M-PEG, CN-PEG, COOH-PEG, or PT-PEG. (1) sodium/naphthalene, THF, room temperature; (2) ethyl bromoacetate, TEA, THF, room temperature; (3) sodium hydroxide solution, reflux; (4) AC, TEA, THF, 10 min room temperature; (5) sodium ethoxide (or sodium metal), CH₂Cl₂, room temperature; (6) acrylonitrile room temperature; (7) AC, TEA, THF, 10 min room temperature; (8) TEA, thionyl bromide, toluene, reflux; (9) p-toluenesulfonylchloride, TEA, CH₂Cl₂, room temperature; (10) AC, TEA, THF, 10 min room temperature; (11) potassium phthalimide, CH₂Cl₂, reflux; (12) AC, TEA, THF, 10 min room temperature

FIGS. 2A and 2B. HPLC chromatogram of (2A) evaporative light scattering detector signals and (2B) UV signals at 254 nm for PEGdiols and various XPEGmA. Samples were analyzed with a reverse phase HPLC system (10% to 100% acetonitrile at a flow rate of 1 ml/min in 30 min with Jordi 500 A column on a Gilson system) coupled to UV/Vis (200 nm and 254 nm), photodiode array, and evaporative light scattering detectors.

FIGS. 3A and 3B. Surface hydrophilicity of the XPEGmA-co-Ac-co-TMPTA network containing XPEGmA of various concentration, terminal moiety, and molecular weight. (3A) 2 KDa XPEGmA and (3B) 5 KDa XPEGmA. Legend: ♦=M-PEG; ▪=CN-PEG; ▴=COOH-PEG; and ●=PT-PEG.

FIG. 4. A schematic representation of hydrogels according to the present invention.

FIG. 5. Graph depicting representative swelling/degradation kinetics. Time in hours is shown on the X-axis; swelling ratio is shown on the Y-axis. Key: G, 0.01% glutaraldehyde cross-linked=♦; 10% PG, 0.01% glutaraldehyde cross-linked=▪; 40% EG, 0.01% glutaraldehyde cross-linked=▴.

FIG. 6. A schematic representation of an interpenetrating network hydrogel according to the present invention.

FIG. 7. A schematic illustration of how the TMPTA networks having COOH groups as grafting sites for peptides are fabricated.

### DETAILED DESCRIPTION OF THE INVENTION

Poly(alkylene glycols), such as poly(ethylene glycol) (PEG), are employed extensively in a number of medical and pharmaceutical fields due to their low toxicity, good biocompatibility, and excellent solubility⁽¹⁻⁵⁾. For sake of expository brevity, the following description shall be limited to gels modified by bifunctional poly(ethylene glycol) molecules. The invention, however, will function with equal success using any poly(alkylene glycol).

Thus, it is preferred that the bifunctional modifier comprise a poly(alkylene glycol) of the formula: 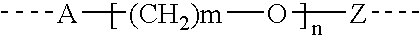
where “m” is an integer of from 2 to 8; “n” is an integer equal to or greater than 100; and “A” and “Z” are as described above. In the preferred embodiment, “m” equals 2 and “n” is sufficiently large to yield a PEG molecule having a molecular weight of roughly 100,000 Da. Thus, it is preferred that “n” is greater than 2,000. The “n” substituent may also be sufficiently large to yield a PEG molecule having a molecular weight greater than 1×10⁶ Da, in which case “n” is greater than roughly 20,000.

While having good biocompatibility and solubility, the hydroxyl groups in PEG-diols or monomethoxy-PEGs have very limited chemical activity. The present invention thus is drawn to novel hydrogels that use bifunctional PEGs and hetero-bifunctional PEGs (“hPEGs”) as covalent grafts to modify the physical and biological properties of hydrogels. These bifunctional PEGS having improved reactivity and physicochemical properties can thus be used to modify polymer matrices in general, and proteinaceous matrices in particular, to yield novel hydrogels. These novel hydrogels are useful in wide array of biomaterial and biopharmaceutical compositions and devices that include a hydrogel component, including time-release vehicles, wound dressings and packing, bandages, burn dressings, catamenial devices, diapers, etc.

Currently, the synthesis of hPEGs is classified into two general categories: 1) statistical terminal modification of PEG precursors; and 2) ethylene oxide polymerization methods using special initiators.⁽⁶⁻⁹⁾ Although various hPEGs are currently available commercially (e.g., from Shearwater Corporation, Huntsville, Alabama), their high cost and limited quantity greatly restricts the extensive utilization of such materials by laboratories in developing novel biomaterials for various applications. In developing the present invention, a number of synthetic schemes were developed to produce a library of hPEG compounds based on the statistical terminal modification method.

A distinct benefit of the various reaction schemes is that they use as a starting material commercially-available PEG-diols. PEG-diol is available in a host of different molecular weights, and from a large number of international suppliers (including Shearwater Corporation).

Moreover, the synthetic strategy is streamlined so modifications to various intermediates results in the formulation of different hPEG products.

Using the hPEGs of the present invention, polymer networks having diverse physicochemical and surface properties were developed. These networks can be used to study cell-material interaction.⁽¹⁰⁻¹³⁾

In the Examples that follow, hPEGs were utilized to modify a polymer matrix to yield novel hydrogels. The effect of hPEG concentration, molecular weight, and terminal chemical functionality on the surface hydrophobicity and cell interaction with the hydrogels was investigated and is presented in the Examples. Multiple heterogeneous PEG modifications (e.g., carboxylic acids of the poly-acrylic acid backbone and the functional group at the dangling terminus of hPEG grafted at the pendent chain configuration) can be employed to bind several distinct types of biofunctional molecules such as peptides and pharmaceutics to the hydrogel.

These components therefore are highly useful as functional wound dressings. In the preferred embodiment, the polymer matrix is a modified gelatin. The use of gelatin is not incidental. Gelatin is a well-characterized, FDA approved, biodegradable biomaterial. Thus, hydrogels made from modified gelatin are likely to pass regulatory muster due to the known safety of gelatin.

The hydrophilicity and porosity of gelatin was modified using ampholytic moieties such as ethylenediaminetetracetic dianhydride (EDTAD). The resulting polymer backbone can be cross-linked with small amounts of glutaraldehyde and subsequently loaded with pharmaceutical agents such as antibiotic drugs. The water-uptake, swelling, degradation, and drug release kinetics of the resulting hydrogel can be controlled by varying the amount of cross-linking and the extent of EDTAD modification.

To improve its biocompatibility and mechanical properties, the hydrogel was then grafted with various hPEGs, as described hereinbelow.

To investigate the functional properties of these novel biomaterials, the interaction of hPEGs, hPEG-modified gelatin hydrogels, and synthetic polymer networks containing human white blood cells and fibroblasts were examined, both in vitro and in vivo. The terminal group of the hPEGs has also been used to link bioactive peptides to the hydrogel matrix, thereby to control the interaction of host cells such as white blood cells and to enhance favorable biological interactions. It has been demonstrated in the Examples that the molecular interaction of several bioactive oligopeptides in modulating white blood cell behavior and host interaction in vitro and in vivo can thus be modified.

The resulting hydrogels can be used a functional wound dressings, bandages, and the like. These functional wound dressings are suitable for use both internally and externally. The gelatin-hPEG hydrogels of the present invention have been tested in a subcutaneous caged implant model.

One notable aspect of the hydrogels of the present invention is that the polymer constructs are not physical blends. The present hydrogels are chemically and physically homogenous and can be tailored to suit a particular clinical endpoint requirement. The hydrogel is mechanically stable because the components are covalently bonded together. Additionally, the hydrophilicity and flexibility of the porous hydrogel accommodates the absorption of wound exudate, blood, etc., and assists in the final removal of material from the wound site.

The nature of gelatin and the porosity of the construct also facilitates the exchange of gases and promotes rapid healing. Most importantly, the presence of hPEG-conjugated bioactive compounds within the hydrogel matrix itself adds qualitative value and control to the wound healing process.

As described hereinbelow, a synthetic scheme was developed to created a library of heterobifunctional PEGs (hPEGs) having two distinct terminal moieties. The hPEGs were then used to make modified polymer hydrogels having various surface and physicochemical properties. Extensive NMR and HPLC analyses confirmed the chemical structure of hPEG. The hydrophilicity of the polymer network was predominantly dependent on the hPEG concentration, with the molecular weight of the starting, unmodified PEG and the terminal functional groups also playing roles. Adherent human fibroblast density on the hydrogels remained constant with increasing hPEG concentration in the gel formulation but decreased rapidly on hydrogels containing 0.8 to 1.25 g/ml of hPEGs. This trend was independent of the hPEG terminal moiety and molecular weight. No adherent cells were observed on all sample gels containing 2.5 g/ml or more of hPEGs.

Abbreviations and Definitions:

“Ac”=acrylic acid

“AC”=acryloyl chloride (CAS No. 814-68-6)

“CHD”=chlorhexidine digluconate

“CN-PEG”=α-cyanoethyl-ω-acrylate-PEG

“COOH-PEG”=α-carboxyl-ω-acrylate-PEG

“EDTAD”=ethylene diaminetetracetic dianhydride

“hPEG”=heterobifunctional PEG

“IPN”=interpenetrating network hydrogels

“mPmA”=α-methyl-ω-aldehyde-PEG

“mPEG”=α-methoxy-PEG

“M-PEG”=α-methyl-ω-acrylate-PEG

“PEG” and “PEG diol”=polyethylene glycol

“PEGdA”=PEG-diacrylate

“PEG dial”=α-aldehyde-ω-aldehyde-PEG

“PT-PEG”=α-phthalimide-ω-acrylate-PEG

“TEA”=triethylamine

“THF”=tetrahydrofuran

“TMPTA”=trimethylolpropane triacrylate (i.e., 2-ethyl-2-(hydroxymethyl)-1,3-propanediol triacrylate, CAS No. 15625-89-5)

“XPEGmA”=hPEG with acrylate ω-terminal and α-terminal of different moiety

The term “alkyl,” by itself or as part of another substituent, means, unless otherwise stated, a fully saturated, straight, branched chain, or cyclic hydrocarbon radical, or combination thereof, and can include di- and multi-valent radicals, having the number of carbon atoms designated (e.g., C₁-C₁₀ means from one to ten carbon atoms, inclusive). Examples of alkyl groups include, without limitation, methyl, ethyl, n-propyl, isopropyl, n-butyl, t-butyl, isobutyl, sec-butyl, cyclohexyl, (cyclohexyl)ethyl, cyclopropylmethyl, and homologs and isomers thereof, for example, n-pentyl, n-hexyl, n-heptyl, n-octyl, and the like. The term “alkyl,” unless otherwise noted, also includes those derivatives of alkyl defined in more detail below as “heteroalkyl” and “cycloalkyl.”

The term “alkenyl” means an alkyl group as defined above containing one or more double bonds. Examples of alkenyl groups include vinyl, 2-propenyl, crotyl, 2-isopentenyl, 2-(butadienyl), 2,4-pentadienyl, 3-(1,4-pentadienyl), etc., and the higher homologs and isomers.

The term “alkynyl” means an alkyl or alkenyl group as defined above containing one or more triple bonds. Examples of alkynyl groups include ethynyl, 1- and 3-propynyl, 3-butynyl, and the like, including the higher homologs and isomers.

The terms “alkylene,” “alkenylene,” and “alkynylene,” alone or as part of another substituent means a divalent radical derived from an alkyl, alkenyl, or alkynyl group, respectively, as exemplified by —CH₂CH₂CH₂CH₂—.

Typically, alkyl, alkenyl, alkynyl, alkylene, alkenylene, and alkynylene groups will have from 1 to 24 carbon atoms. Those groups having 10 or fewer carbon atoms are preferred in the present invention. The term “lower” when applied to any of these groups, as in “lower alkyl” or “lower alkylene,” designates a group having eight or fewer carbon atoms.

“Substituted” refers to a chemical group as described herein that further includes one or more substituents, such as lower alkyl, aryl, acyl, halogen (e.g., alkylhalo such as CF₃), hydroxy, amino, alkoxy, alkylamino, acylamino, thioamido, acyloxy, aryloxy, aryloxyalkyl, mercapto, thia, aza, oxo, both saturated and unsaturated cyclic hydrocarbons, heterocycles and the like. These groups may be attached to any carbon or substituent of the alkyl, alkenyl, alkynyl, alkylene, alkenylene, and alkynylene moieties. Additionally, these groups may be pendent from, or integral to, the carbon chain itself.

The term “heteroalkyl,” by itself or in combination with another term, means, unless otherwise stated, a stable, saturated or unsaturated, straight, branched chain, or cyclic hydrocarbon radical, or combinations thereof, consisting of the stated number of carbon atoms and from one to three heteroatoms selected from the group consisting of O, N, Si, and S, and wherein the nitrogen and sulfur atoms may optionally be oxidized and the nitrogen heteroatom(s) may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as in —CH₂—NH—O—CH₃ and —CH₂—O—Si(CH₂)₃. Explicitly included within the term “heteroalkyl” are those radicals that could also be described as “heteroalkylene” (i.e., a divalent radical, see next paragraph), and “heterocycloalkyl” (i.e., containing a cyclic group). The term “heteroalkyl” also explicitly includes unsaturated groups (i.e., heteroalkenyls and heteroalkynyls).

The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

The term “aryl” is used herein to refer to an aromatic substituent, which may be a single aromatic ring or multiple aromatic rings which are fused together, linked covalently, or linked to a common group such as a diazo, methylene or ethylene moiety. The common linking group may also be a carbonyl as in benzophenone. The aromatic ring(s) may include, for example phenyl, naphthyl, biphenyl, diphenylmethyl and benzophenone, among others. The term “aryl” encompasses “arylalkyl” and “substituted aryl.” For phenyl groups, the aryl ring may be mono-, di-, tri-, tetra-, or penta-substituted. Larger rings may be unsubstituted or bear one or more substituents.

“Substituted aryl” refers to aryl as just described including one or more functional groups such as lower alkyl, acyl, halogen, alkylhalo (e.g., CF₃), hydroxy, amino, alkoxy, alkylamino, acylamino, acyloxy, phenoxy, mercapto, and both saturated and unsaturated cyclic hydrocarbons which are fused to the aromatic ring(s), linked covalently or linked to a common group such as a diazo, methylene, or ethylene moiety. The linking group may also be a carbonyl such as in cyclohexyl phenyl ketone. The term “substituted aryl” encompasses “substituted arylalkyl.” The term “acyl” is used to describe a ketone substituent, —C(O)R, where R is substituted or unsubstituted alkyl, alkenyl, alkynyl, or aryl as defined herein. The term “carbonyl” is used to describe an aldehyde substituent. The term “carboxy” refers to an ester substituent or carboxylic acid, i.e., —C(O)O— or —C(O)—OH.

The term “halogen” or “halo” is used herein to refer to fluorine, bromine, chlorine and iodine atoms.

The term “hydroxy” is used herein to refer to the group —OH.

The term “amino” is used to designate NRR′, wherein R and R are independently H, alkyl, alkenyl, alkynyl, aryl or substituted analogs thereof. “Amino” encompasses “alkylamino,” denoting secondary and tertiary amines, and “acylamino” describing the group RC(O)NR′.

The term “alkoxy” is used herein to refer to the —OR group, where R is alkyl, alkenyl, or alkynyl, or a substituted analog thereof. Suitable alkoxy radicals include, for example, methoxy, ethoxy, t-butoxy, etc. The term “alkoxyalkyl” refers to ether substituents, monovalent or divalent, e.g. —CH₂—O—CH₃ and —CH₂—O—CH₂—.

The term “gelatin” as used herein means any and all kinds of gelatin, of any type (e.g., Type A from pork, with an isoelectric point between about 7.0 and 9.0, and Type B from beef with an isoelectric point of approximately 5.0), from any source, of any bloom value, acid- or alkaline-treated, etc., without limitation. The “bloom strength” of a gelatin is defined as the force required for a plunger of defined shape and size to make a 4 mm depression in a gel that has been prepared at 6.67% w/w concentration and chilled at 10° C. in a bloom jar for 16-18 hours. The force is recorded in grams. Commercially, gelatin is available from a host of commercial suppliers. At commodity amounts and prices, gelatin is generally available with bloom strengths ranging from about 50-300 bloom. Such gelatins are available from, for example, Leiner Davis Gelatin, a wholly-owned subsidiary of Goodman Fielder Ingredients of Sydney, Australia. Gelatins having bloom values outside this range are also available as specialty chemicals and are included within the scope of the term “gelatin.” For example a zero bloom (non-gelling) gelatin is available from Great Lakes Gelatin Co., Grayslake, Ill.

Likewise, the term “collagen” as used herein means any and all kinds of collagen, of any type, from any source, without limitation. Cross-linked collagen, esterified collagen, and chemically-modified collagen, such as that taught by U.S. Pat. No. 4,390,519, are included with the term “collagen.”

The term “polymer matrix” encompasses any type of polymer matrix that can function as a hydrogel, including, without limitation, gelatin, calcium alginate, calcium/sodium alginate, collagen, oxidized regenerated cellulose, carboxymethylcellulose, amino-modified celluloses, such as 6-deoxy-6-(4-aminophenyl)-amino-2(3)-O-tosylcellulose, whey protein gels, and the like.

The term “photopolymerizable acrylate” refers to any acrylate-containing molecule capable of being photopolymerized, without limitation. Expressly included within this definition are bis-diacrylate-PEGs, i.e., poly(alkylene glycol) molecules having an α-acrylate moiety and an ω-acrylate moiety. TMPTA is also a photopolymerizable acrylate.

Modified PEGs:

Commercial PEG-diols can be purchased essentially as a commodity item, in large amounts and at relatively inexpensive prices. The first step in modifying the α- and ω-termini of the PEG-diols is to convert them in aldehyde groups. This is very easily accomplished by treating the PEG-diol with acetic anhydride: 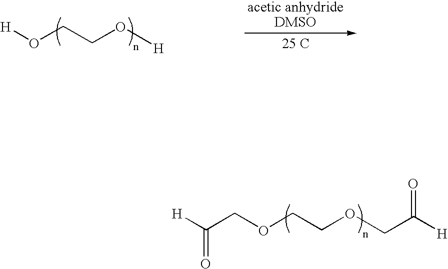
The reaction is very facile and quantitative.

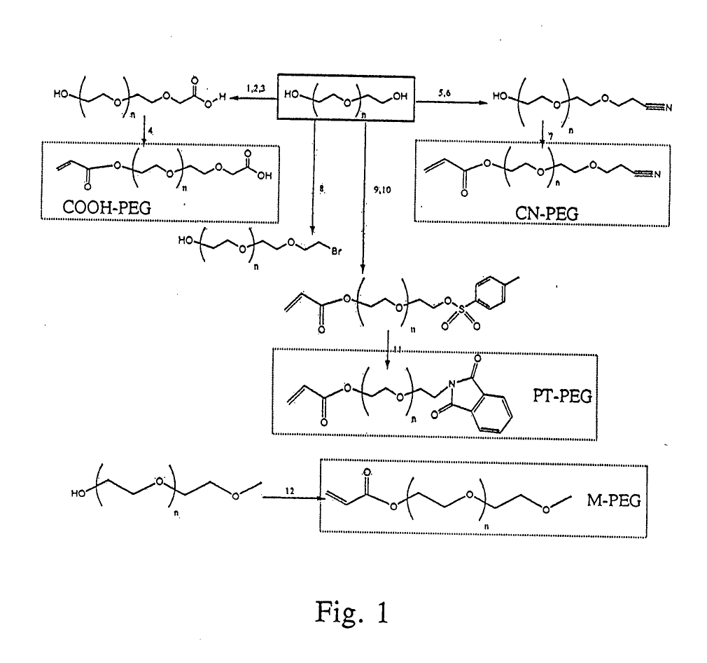

With the PEG-dialdehyde in hand, the molecule can be further modified using any of the routes shown in FIG. 1, among many others. For example, as shown in FIG. 1, the PEG-diol can be converted into an α-hydroxy-ω-carboxy-PEG, which can then be converted into an α-acrylate-ω-carboxy-PEG. Or the PEG-diol can be converted into a α-hydroxy-ω-cyanoethyl-PEG, which can then, in turn, be converted into a α-acrylate-ω-cyanoethyl-PEG.

The PEG-diol can be directly converted, by simple halogenation of the hydroxy group to α-hydroxy-ω-halo-PEG. The PEG diol can also be tosylated and acrylated to thereby yield α-acrylate-ω-tosylated-PEG. The tosyl group can be exchanged for a succinimidyl or phthalimidyl or other nitrogen-containing heterocycle group. α-Hydroxy-ω-methoxy-PEG can be converted directly into α-acrylate-ω-methoxy-PEG. See FIG. 1. (See also Hem & Hubbell,(1998) J. Biomed. Mater. Res. 39:266-276; Morpurgo et al. (1996) App. Biochem. Biotech. 56:59-72; and Abuchowski et al. (1984) Cancer Biochem. Biophys. 7:175-186.)

Thus, for example, α-hydroxy-ω-glutarate-PEG can be synthesized by treating a PEG-diol with glutaric anhydride and glutaric acid in THF with gentle heating:

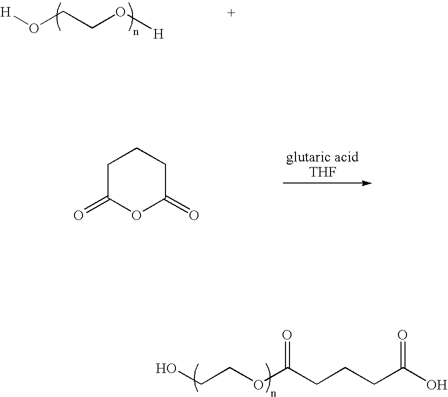

The glutaric anhydride and the glutaric acid are added and the solution gently heated to 55° C. The solution is maintained at that temperature, with stirring, for one day. The solution is then cooled to room temperature and filtered. The filtrate is then precipitated in cold hexane, the resulting precipitate is then removed by filtration, and dried in a vacuum to yield the desired product, generally a mixture of PEG-bis-glutarate and α-hydroxy-ω-glutarate-PEG. The two can be separated chromatographically (see the Examples).

The glutarate group can be further reacted to add a nitrogen-containing heterocycle, such as a succinimidyl group by reacting the α-hydroxy-ω-glutarate-PEG with N-hydroxy-succinimide in the presence of a water-soluble carbodiimide:

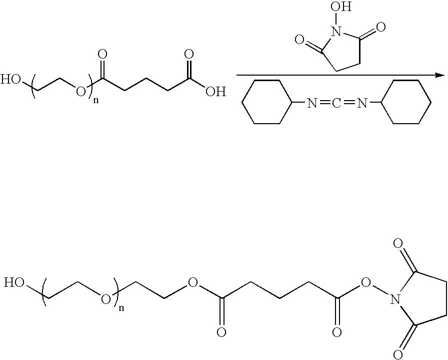

The N-hydroxy-succinimide is added and the solution cooled to 0° C. The dicyclohexylcarbodiimide (DCC) is added dropwise and the solution stirred for one day and filtered. The filtrate is precipitated by adding cold hexane. The resulting precipitate is filtered and dried in a vacuum. This yields the desired product, generally a mixture of PEG-bis-N-succinimidylglutarate and α-glutarate-ω-succinimidylglutarate-PEG (or α-hydroxy-ω-succinimidylglutarate-PEG, depending upon the starting material chosen). The two can be separated chromatographically (see the Examples).

The α-hydroxy-ω-succinimidylglutarate can be further reacted to yield α-acrylate-ω-succinimidylglutarates by reacting the α-hydroxy-ω-succinimidylglutarate with acrylic acid in the presence of TEA.

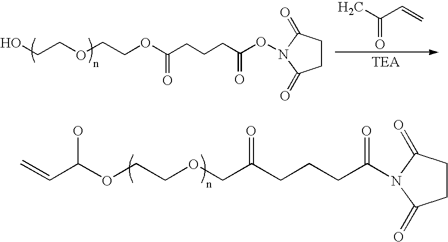

The PEG molecules may also be modified to introduce other amide bonds into the molecule. The formation of an amide bond is, of course, extremely useful in modifying the PEG molecule to contain an amino acid, peptide, or protein terminus. Thus, for example α-succinimidylglutarate-ω-tryptophanylglutarate PEG can be synthesized by dissolving the peptide or amino acid in 0.1 M 2-(N-morpholino)-ethanesulfonic acid (MES) at 0° C. α,ω-Bis-N-succinimidylglutarate-PEG is added dropwise to the solution with constant stirring. The reaction is allowed to continue at 0° C. for 1 hour and then allowed to come to room temperature with constant stirring for 4 hours. The reaction solution is then dialyzed against 50 volumes of deionized water and the resulting solution lyophilized. This yields the desired α-N-succinimidylglutarate-ω-tryptophanylglutarate in rougly 40% yield.

The modified PEGs can be attached to a polymer matrix containing amino-reactive groups using the same procedure as in the previous paragraph, thereby grafting the modified PEG to the amino-reactive groups of the polymer matrix. See also the Examples. In short, the mono- or dialdehyde-PEG is first dissolved in water. A separate aqueous solution of NaCNBH₃ is also prepared. The two solutions are then added simultaneously to a dilute (5%) solution of gelatin in water. The reaction is allowed to proceed overnight with gentle heating (50 to 60° C.). The modified gelatin is then separated by filtration.

Using these various synthetic schemes, the following modified PEG molecules have been made and used to modify gelatin to yield novel hydrogels that fall within the scope of the present invention:

Series 1: Alpha-Methoxy Heterobifunctional

PEG Derivatives: 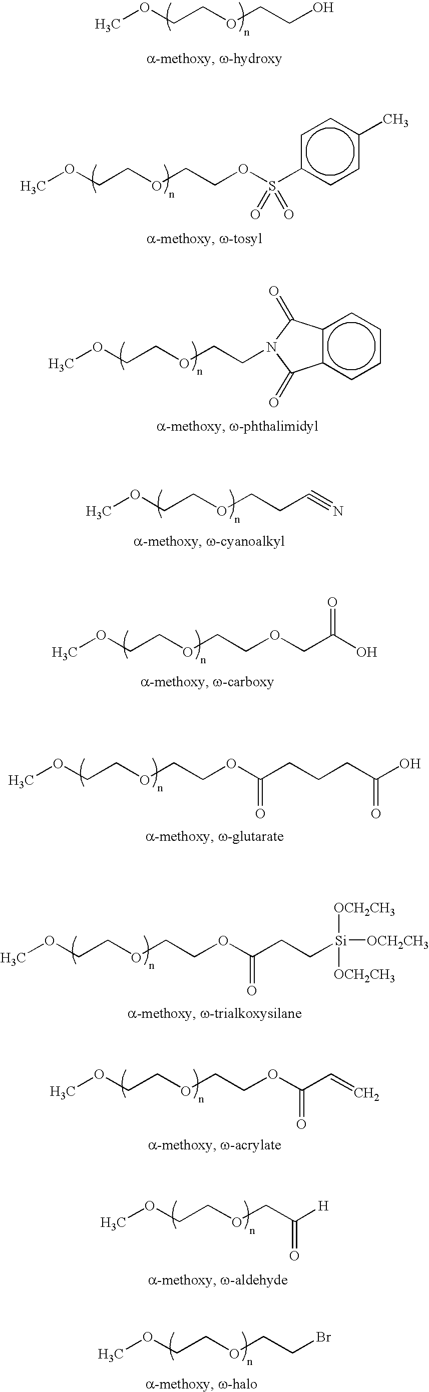 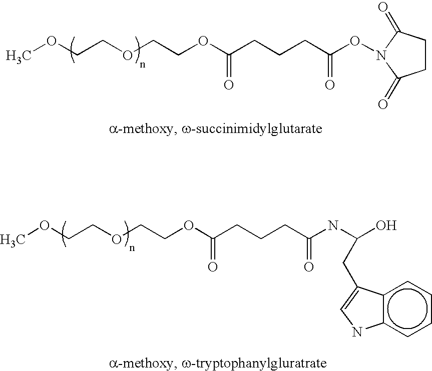
Series 1 Chemistry:

1.1. α-Methoxy, ω-hydroxy-PEG is commercially available (Shearwater).

1.2. To synthesize α-methoxy, ω-tosyl PEG, PEG (1 eq.) was dissolved in dry methylene chloride MC followed by addition of p-toluenesulfonylchloride (1 eq.) and triethylamine (1 eq.). The solution was stirred at room temperature for 48 hr, precipitated in cold hexane, collected by filtration and dried in vacuum oven for 24 hr.

1.3. To synthesize α-methoxy, ω-phthalimidyl PEG, α-methoxy, ω-tosyl PEG (1 eq.) from series 1-2 and potassium phthalimide (1.2 eq.) were dissolved in toluene and stirred at 50° C. for 20 hr. The solution was cooled down, filtered and the filtrate was precipitated in cold hexane, collected by filtration and dried in vacuum oven for 24 hr.

1.4. To synthesize α-methoxy, ω-cyanoalkyl PEGs, PEG (1 eq.) was dissolved in dry MC solution followed by the addition of fine sodium metal (1.5 eq.) and stirred for 12 hr at room temperature. Excess amount of acrylonitrile was added to the solution, stirred for 12 hr, filtered, and dried by rotary evaporation.

1.5. To synthesize α-methoxy, ω-carboxy PEG, PEG (1 mol) was dissolved in dry THF. Sodium (1.2 eq.) and naphthalene (1.2 eq.) were dissolved in dry THF and stirred under argon for 1 hr. The sodium/naphthalene solution was added dropwise to the PEG solution and the solution was stirred under argon for 4 hr. Ethyl bromoacetate (1.2 eq.) was then added and the solution was stirred under argon for 12 hr. The solution was filtered and the filtrate was precipitated in cold hexane, collected by filtration and dried in vacuum oven for 24 hr. The dried substance was dissolved in deionized water followed by addition of sodium hydroxide (1 eq.). The solution was stirred at 40° C. for two hr, extracted by MC or two times and evaporated by rotary evaporation.

1.6. To synthesize α-methoxy, ω-glutarate PEG, PEG (1 eq.) was dissolved in dry THF followed by addition of glutaric anhydride (1.5 eq.) and glutaric acid (0.001 eq.). The solution was stirred at 70° C. for 48 hr, cooled down, precipitated in cold hexane, collected by filtration and dried in vacuum oven for 24 hr.

1.7. To synthesize α-methoxy, ω-triethoxysilane PEG, α-methoxy, ω-acrylate PEG (1 eq.) from series 2-8 was dissolved in dry THF followed by addition of triehyoxysilane (5 eq.) and chloroplatinic acid (a grain). The solution was stirred at 60° C. for 48 hr, cooled down, precipitated in cold hexane, collected by filtration and dried in vacuum oven for 24 hr.

1.8. To synthesize α-methoxy, ω-acrylate PEG, PEG (1 eq.) was dissolved in dry THF followed by the addition of acryloyl chloride (2 eq.) and triethylamine (2.2 eq.). The solution was stirred at room temperature for 2 hr, filtered and the filtrate was precipitated in cold hexane, collected by filtration and dried in vacuum oven for 24 hr.

1.9. To synthesize α-methoxy, ω-aldehyde PEG, PEG (1 eq.) was dissolved in DMSO and the solution was added dropwise to the acetic anhydride (20 eq.) and stirred at room temperature for 2 hr. Ether was then added to the solution and stirred for 5 min at room temperature and placed in the −20° C. freezer for 5 minutes to precipitate. The precipitate was collected by filtration and then dissolved in minimal amounts of methylene chloride and reprecipitated likewise twice by ether. The precipitate was dried in vacuum oven for 24 hr.

1.10. To synthesize α-methoxy, ω-halo PEG, PEG (1 eq.) was dissolved in toluene followed by addition of triethylamine (1.2 eq.). The solution was stirred at 60° C. for 30 min followed by addition of thionyl bromide (1.2 eq.) and stirred at 60° C. for 1 hr. The hot solution was filtered through celite and the filtrate was kept in refrigerator at −4° C. for 24 hr. The precipitate was collected by filtration and dried in vacuum oven for 24 hr.

1.11. To synthesize α-methoxy, ω-succinimidylglutarate-PEG, α-methoxy, ω-glutarate PEG (1 eq.) from series 1.6 and dicyclohexylcarbodiimide (DCC 1.2 eq.) was dissolved in dry THF respectively. N-hydroxy succinimide (1.2 eq.) was added to the PEG solution followed by dropwise addition of DCC solution. The mixture solution was stirred at room temperature for 6 hr, filtered and the filtrate was precipitated in cold hexane, collected by filtration and dried in a vacuum oven for 3 days and then stored under argon at −4° C. in the refrigerator.

1.12. To synthesize α-methoxy, ω-succinimidylglutrate PEG, α-methoxy, ω-succinimidylglutrate PEG (1 eq.) from series 1.11 was dissolved in DMF followed by addition of tryptophan (1.5 eq.). The solution was stirred under argon for 24 hrs, dialyzed in deionized water and dried by lyophilizer for 3 days.
Series 2: Alpha-Hydroxy Heterobifunctional PEG Derivatives: 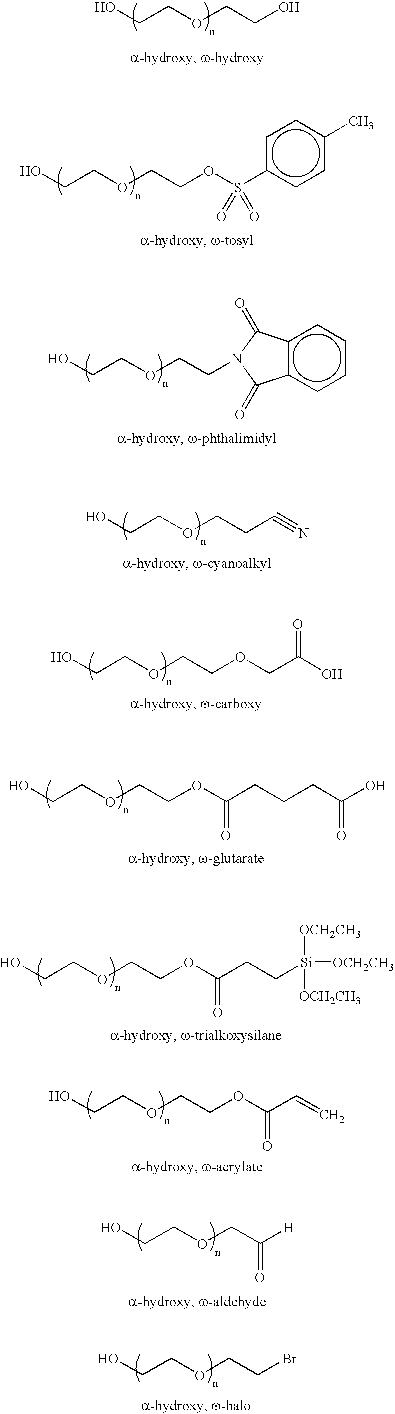 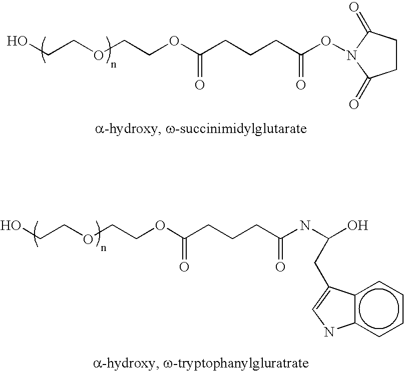
Series 2 Chemistry:

2.1. PEG is commercially available.

2.2. To synthesize α-hydroxy, ω-tosyl PEG, PEG (1 eq.) was dissolved in dry methylene chloride followed by addition of p-toluenesulfonylchloride (1 eq.) and triethylamine (1 eq.). The solution was stirred at room temperature for 48 hr, precipitated in cold hexane, collected by filtration and dried in vacuum oven for 24 hr.

2.3. To synthesize α-hydroxy, ω-phthalimidyl PEG, α-hydroxy, ω-tosyl PEG (1 eq.) from series 2.2 and potassium phthalimide (1.2 eq.) were dissolved in toluene and stirred at 50° C. for 20 hr. The solution was cooled down, filtered and the filtrate was precipitated in cold hexane, collected by filtration and dried in a vacuum oven for 24 hr.

2.4. To synthesize α-hydroxy, ω-cyanoalkyl PEGs, PEG (1 eq.) was dissolved in dry methylene chloride solution followed by the addition of fine sodium metal (1.2 eq.) and stirred for 12 hr at room temperature. Excess amount of acrylonitrile was added to the solution, stirred for 12 hr, filtered, and dried by rotary evaporation.

2.5. To synthesize α-hydroxy, ω-carboxy PEG, PEG (1 mol) was dissolved in dry THF, sodium (1.2 eq.) and naphthalene (1.2 eq.) were dissolved in dry THF and stirred under argon for 1 hr. The sodium/naphthalene solution was added dropwise to the PEG solution, the solution was stirred under argon for 4 hr. Ethyl bromoacetate (1.2 eq.) was then added and the solution was stirred under argon for 12 hr. The solution was filtered and the filtrate was precipitated in cold hexane, collected by filtration and dried in vacuum oven for 24 hr. The dried substance was dissolved in deionized water followed by addition of sodium hydroxide (1 eq.). The solution was stirred at 40° C. for two hr, extracted by methylene chloride for two times and evaporated by rotary evaporation.

2.6. To synthesize α-hydroxy, ω-glutarate PEG, PEG (1 eq.) was dissolved in dry THF followed by addition of glutaric anhydride (1.5 eq.) and glutaric acid (0.001 eq.). The solution was stirred at 70° C. for 48 hr, cooled down, precipitated in cold hexane, collected by filtration and dried in vacuum oven for 24 hr.

2.7. To synthesize α-hydroxy, ω-triethoxysilane PEG, α-hydroxy, ω-acrylate PEG (1 eq.) from series 2.8 was dissolved in dry THF followed by addition of triehyoxysilane (5 eq.) and chloroplatinic acid (a grain). The solution was stirred at 60° C. for 48 hr, cooled down, precipitated in cold hexane, collected by filtration and dried in vacuum oven for 24 hr.

2.8. To synthesize α-hydroxy, ω-acrylate PEG, PEG (1 eq.) was dissolved in dry THF followed by the addition of acryloyl chloride (1.5 eq.) and triethylamine (1.7 eq.). The solution was stirred at room temperature for 2 hr, filtered and the filtrate was precipitated in cold hexane, collected by filtration and dried in vacuum oven for 24 hr.

2.9. To synthesize α-hydroxy, ω-aldehyde PEG, PEG (1 eq.) was dissolved in DMSO and the solution was added dropwise to the acetic anhydride (20 eq.) and stirred at room temperature for 2 hr. Ether was then added to the solution and stirred for 5 min at room temperature and placed in the −20° C. freezer for 5 minutes to precipitate. The precipitate was collected by filtration and then dissolved in minimal amounts of methylene chloride and reprecipitated likewise twice by ether. The precipitate was dried in vacuum oven for 24 hr.

2.10. To synthesize α-hydroxy, (ω-halo PEG, PEG (1 eq.) was dissolved in toluene followed by addition of triethylamine (1.2 eq.). The solution was stirred at 60° C. for 30 min followed by addition of thionyl bromide (1.2 eq.) and stirred at 60° C. for 1 hr. The hot solution was filtered through celite and the filtrate was kept in refrigerator at −4° C. for 24 hr. The precipitate was collected by filtration and dried in vacuum oven for 24 hr.

2.11. To synthesize α-hydroxy, ω-succinimidylglutrate PEG, α-hydroxy, ω-glutarate PEG (1 eq.) resulted from series 2.6 and dicyclohexylcarbodiimide (DCC 1.2 eq.) were dissolved in dry THF respectively. N-hydroxy succinimide (1.2 eq.) was added to the PEG solution followed by dropwise addition of DCC solution. The mixture solution was stirred at room temperature for 6 hr, filtered and the filtrate was precipitated in cold hexane, collected by filtration and dried in vacuum oven for 3 days and then stored under argon at −4° C. in the refrigerator.

2.12. To synthesize α-hydroxy, ω-tryptophanylglutrate PEG, a-hydroxy, w-succinimidylglutrate PEG (1 eq.) from series 2.11 was dissolved in DMF followed by addition of tryptophan (1.5 eq.). The solution was stirred under argon for 24 hrs, dialyzed in deionized water and dried by lyophilizer for 3 days.
Series 3: Alpha-Acrylate Heterobifunctional PEG Derivatives: 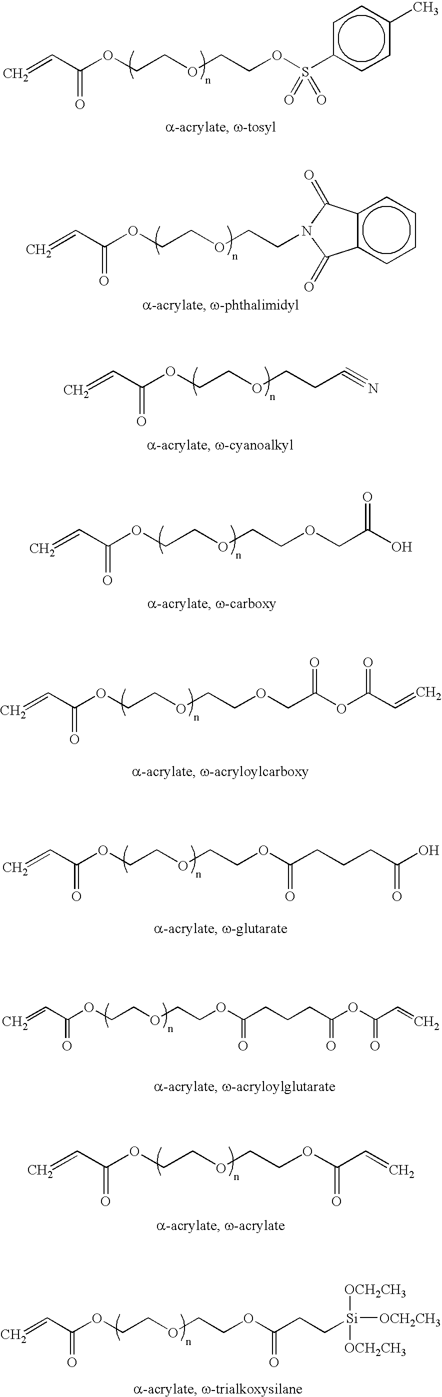 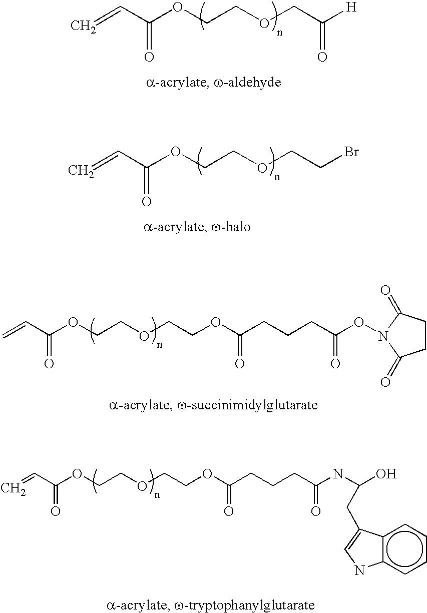
Series 3 Chemistry:

3.1. To synthesize α-acylate, ω-tosyl PEG, α-hydroxy, ω-tosyl PEG (1 eq.) from series 2.2 was dissolved in dry THF followed by addition of acryloyl chloride (2 eq.) and triethylamine (2.2 eq.). The solution was stirred at room temperature for 2 hr, filtered and the filtrate was precipitated in cold hexane, collected by filtration and dried in vacuum oven for 24 hr.

3.2. To synthesize α-acylate, ω-phthalimidyl PEG, α-acylate, ω-tosyl PEG (1 eq.) from series 3.1 and potassium phthalimide (2 eq.) were dissolved in toluene and stirred at 50° C. for 20 hr. The solution was cooled down, filtered and the filtrate was precipitated in cold hexane, collected by filtration and dried in vacuum oven for 24 hr.

3.3. To synthesize α-acylate, ω-cyanoalkyl PEGs, α-hydroxy, ω-acrylate PEG (1 eq.) from series 2.8 was dissolved in dry methylene chloride solution followed by the addition of fine sodium metal (1.5 eq.) and stirred for 12 hr at room temperature. Excess amount of acrylonitrile was added to the solution, stirred for 12 hr, filtered, and dried by rotary evaporation.

3.4. To synthesize α-acylate, ω-carboxy PEG, α-hydroxy, ω-carboxy PEG (1 eq.) from series 2.5 was dissolved in dry THF followed by addition of acryloyl chloride (1.2 eq.) and triethylamine (1.5 eq.). The solution was stirred at room temperature for 2 hr, filtered and the filtrate was precipitated in cold hexane, collected by filtration and dried in vacuum oven for 24 hr.

3.5. To synthesize α-acylate, ω-acryloylcarboxy PEG, ω-carboxy PEG (1 eq.) from series 2.5 was dissolved in dry THF followed by addition of acryloyl chloride (3 eq.) and triethylamine (3.5 eq.). The solution was stirred at room temperature for 2 hr, filtered and the filtrate was precipitated in cold hexane, collected by filtration and dried in vacuum oven for 24 hr.

3.6. To synthesize α-acylate, (ω-glutarate PEG, α-hydroxy, ω-glutarate PEG (1 eq.) from series 2.6 was dissolved in dry THF followed by addition of acryloyl chloride (1.2 eq.) and triethylamine (1.5 eq.). The solution was stirred at room temperature for 2 hr, filtered and the filtrate was precipitated in cold hexane, collected by filtration and dried in vacuum oven for 24 hr.

3.7. To synthesize α-acylate, ω-acryloylglutarate PEG, α-hydroxy, ω-glutarate PEG (1 eq.) from series 2.6 was dissolved in dry THF followed by addition of acryloyl chloride (3 eq.) and triethylamine (3.5 eq.). The solution was stirred at room temperature for 2 hr, filtered and the filtrate was precipitated in cold hexane, collected by filtration and dried in vacuum oven for 24 hr.

3.8. To synthesize α-acrylate, ω-acrylate PEG, PEG (1 eq.) was dissolved in dry THF followed by the addition of acryloyl chloride (3 eq.) and triethylamine (3.5 eq.). The solution was stirred at room temperature for 2 hr, filtered and the filtrate was precipitated in cold hexane, collected by filtration and dried in vacuum oven for 24 hr.

3.9. To synthesize α-acylate, ω-triethoxysilane PEG, α-hydroxy, ω-triethoxysilane PEG (1 eq.) from series 2.7 was dissolved in dry THF followed by the addition of acryloyl chloride (1.2 eq.) and triethylamine (1.5 eq.). The solution was stirred at room temperature for 2 hr, filtered and the filtrate was precipitated in cold hexane, collected by filtration and dried in vacuum oven for 24 hr.

3.10. To synthesize α-acylate, ω-aldehyde PEG, α-hydroxy, ω-aldehyde PEG (1 eq.) from series 2.9 was dissolved in dry THF followed by the addition of acryloyl chloride (1.2 eq.) and triethylamine (1.5 eq.). The solution was stirred at room temperature for 2 hr, filtered and the filtrate was precipitated in cold hexane, collected by filtration and dried in vacuum oven for 24 hr.

3.11. To synthesize α-acylate, ω-halo PEG, α-hydroxy, ω-halo PEG (1 eq.) from series 2.10 was dissolved in dry THF followed by the addition of acryloyl chloride (1.2 eq.) and triethylamine (1.5 eq.). The solution was stirred at room temperature for 2 hr, filtered and the filtrate was precipitated in cold hexane, collected by filtration and dried in vacuum oven for 24 hr.

3.12. To synthesize α-acylate, ω-succinimidylglutrate PEG, α-hydroxy, ω-succinimidylglutrate PEG (1 eq.) from series 2.11 was dissolved in dry THF followed by the addition of acryloyl chloride (1.2 eq.) and triethylamine (1.5 eq.). The solution was stirred at room temperature for 2 hr, filtered and the filtrate was precipitated in cold hexane, collected by filtration and dried in vacuum oven for 24 hr.

3.13. To synthesize α-acylate, ω-succinimidylglutrate PEG, α-hydroxy, ω-tryptophanylglutrate PEG (1 eq.) from series 2.12 was dissolved in dry THF followed by the addition of acryloyl chloride (1.2 eq.) and triethylamine (1.5 eq.). The solution was stirred at room temperature for 2 hr, filtered and the filtrate was precipitated in cold hexane, collected by filtration and dried in vacuum oven for 24 hr.

Series 4: Homo-Bifunctional PEG Derivatives:

Modified PEGS wherein the α and the ω termini have the same functional groups can also be fabricated using the same approach. Thus, using the chemistries described herein, bis-acrylate, bis-tosylate, bis-phthalimidyl, bis-cyanoalkyl, bis-carboxylate, bis-acryloylcarboxylate, bis-glutarate, bis-acryloylglutarate, bis-trialkoxysilane, bis-aldehyde, bis-N-succinimidyl, and bis-tryptophanylglutarate derivatives can be fabricated.

Thus, according to the present invention, a polymer matrix, preferably gelatin, is modified to contain one or more of the modified PEG molecules disclosed herein. The PEG molecule may be bis-modified, using the same type of moiety. Or, the α-terminus of the PEG may have a different moiety than the ω-terminus. Both versions of the modified PEG molecules, as incorporated into a hydrogel, fall within the scope of the present invention.

Interpenetrating Network Hydrogels (IPNs):

The above described PEG-modified hydrogels can also be used as a first polymer matrix in an interpenetrating network of two distinct polymer matrices. In this aspect of the invention, the PEG-modified hydrogels as described above are admixed with a polymerizable mixture of monomers. A polymerization reaction is then initiated, whereby the mixture of monomers polymerizes in situ, thereby forming a second polymer matrix that interpenetrates with the first polymer matrix.

It is much preferred that the plurality of monomers that forms the second polymer matrix is polymerizable by a means other than chemical initiation. Chemically polymerizable monomers are, however, within the scope of the invention. In the preferred embodiment, the monomers are photopolymerizable. Thus, the monomers are admixed with the first polymer matrix. The mixture is then exposed to a suitable wavelength of radiation (e.g., infrared, visible, or ultraviolet) that will result in a photo-initiated polymerization reaction. The source for the radiation can be any source that generates radiation of the required wavelength, such as lamps (incandescent, fluorescent, ion discharge, etc.), lasers (CO₂, Ne—Ne, etc.), and light-emitting diodes.

The preferred photopolymerizable monomers are acrylates, diacrylates, and poly(acrylates) (including PEG-acrylates, PEG-diacrylates, and TMPTA), acrylic acid, and acryloyl halides, such as acryloyl chloride, and mixtures thereof. When a plurality of different monomers is admixed with the first polymer matrix, the polymerization reaction will, of course, result in the second polymer matrix being a co-polymer. Thus, the second polymer matrix may comprise a homo-polymer matrix or a co-polymer matrix of any description (e.g., alternating, block, or graft co-polymers).

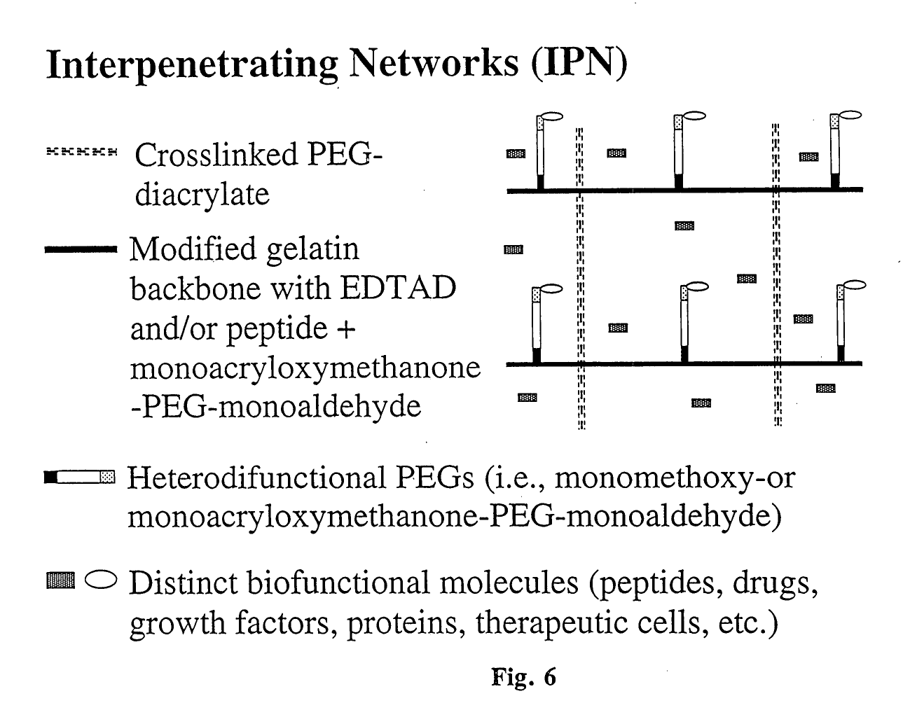

FIG. 6 is a schematic representation of interpenetrating network hydrogels according to the present invention. The gels can contain living cells or pharmcalogically-active agents, or both.

### EXAMPLES

The following Examples are included herein solely to provide a more complete and consistent understanding of the invention disclosed and claimed herein. The Examples do not limit the scope of the invention in any fashion.

### Example 1

### Synthesis and Characterization of Heterobifunctional PEGs

All reagents were purchased from Sigma-Aldrich (St. Louis, Missouri) unless stated otherwise. A summary of the chemical reactions and structure of critical intermediates and final products is presented in FIG. 1.

To synthesize α-methyl-ω-acrylate PEGs (M-PEG), monomethoxy PEGs (2 kDa or 5 kDa, purchased from Fluka, a division of Sigma-Aldrich) were dissolved in dry tetrahydrofuran (THF) solution followed by the addition of triethylamine (TEA, 2 eq.) and acryloyl chloride (AC, 4 eq.)⁽¹⁴⁾ at room temperature under Ar for 10 min, filtered, dried by rotary evaporation, re-dissolved in CH₂Cl₂, and precipitated in cold hexane. The final product was filtered, dried, and stored in vacuo at room temperature.

To synthesize α-cyanoethyl-ω-acrylate-PEGs (CN-PEG), PEG-diols (2 kDa or 5 kDa) (1 eq.) were dissolved in dry CH₂Cl₂ solution followed by the addition of fine sodium metal (2 eq.) stirred for 12 hr at room temperature. An excess amount of acrylonitrile was added into the solution^{(15, 16)}, stirred for 12 hr, filtered, and dried by rotary evaporation. The product thus formed (i.e., α-nitrile-ω-hydroxy-PEG) was dissolved in dry THF, followed by the addition of TEA (2 eq.) and AC (4 eq.). The solution was stirred under Ar for 10 min at room temperature. Triethylammonium chloride was removed by filtration and the solvent was removed by rotary evaporation. The final product was re-dissolved into CH₂Cl₂, precipitated in cold hexane, filtered, and stored in vacuo at room temperature.

To synthesize α-carboxyl-ω-acrylate-PEGs (COOH-PEG), sodium (3.5 eq) in mineral oil was dried, dissolved in dried THF with naphthalene (3.5 eq), and stirred for 1 hr under Ar at room temperature.⁽¹⁷⁾ The sodium/naphthalene solution thus formed was added drop wise into PEG-diols (2 kDa or 5 kDa) (1 eq.) dissolved in dried THF under Ar for 4 hr. Ethyl bromoacetate (4 eq) was added to the ionized PEG solution, stirred for 12 hr, filtered, precipitated in cold hexane, and re-dissolved in distilled water (1 eq) with sodium hydroxide (3 eq)⁽¹⁸⁾, followed by reflux for 24 hr at room temperature. Solvent was removed by rotary evaporation and the solid was re-dissolved in CH₂Cl₂, filtered, precipitated in cold hexane, dried in vacuo. The solid of mainly α-carboxyl-ω-hydroxyl-PEGs (1 eq.) was dissolved in dried THF followed by the addition of TEA (2 eq.) and AC (4 eq), stirred at room temperature for 10 min under Ar, filtered, precipitated in cold hexane, filtered, dried, and stored in vacuo at room temperature.

To synthesize α-phthalimide-ω-acrylate-PEGs (PT-PEG), PEG-diols (2 kDa or 5 kDa) (1 eq.) were dissolved in dry CH₂Cl₂ solution followed by the addition of TEA (4 eq.) and p-toluenesulfonyl chloride (2 eq.)⁽¹⁹⁾ and stirred under Ar for 8 hr at room temperature. Solvent was removed by rotary evaporation to obtain yellowish white solids. This mixture of PEG-diols, α-hydroxyl-ω-tosyl-PEGs, and bis-tosyl-PEG (1 eq.) was dissolved in dry THF, followed by the addition of TEA (2 eq.) and AC (4 eq.), stirred at room temperature under Ar for 10 min, filtered to remove triethylammonium chloride, dried via rotary evaporation to remove solvents, re-dissolved into CH₂Cl₂, and precipitated in cold hexane. The solid product (mainly α-tosyl-ω-acrylate-PEG) was filtered, dried in vacuo, dissolved (1 eq.) in CH₂Cl₂, followed by the addition of potassium phthalimide (3 eq.)⁽²⁰⁾ and refluxed for 18 hr. The solution was filtered, dried via rotary evaporation to remove solvents, re-dissolved into CH₂Cl₂, precipitated in cold hexane, filtered, dried, and stored in vacuo at room temperature.

All intermediates and final products were analyzed with ¹H- and ¹³C-NMR with samples dissolved in CDCl₃ and with a reverse-phase HPLC system (10% to 100% acetonitrile at a flow rate of 1 ml/min in 60 min with a Jordi 500 Å column on a Gilson system) coupled to an automated multi-sample sampler-fraction collector. Detectors included UV/Vis (200 and 254 nm), photodiode array, and evaporative light scattering detectors.

The above-described heterobifunctional PEGs (hPEGs) were employed as a component in the formation of hydrogels to investigate the influence of hPEG concentration, molecular weight, and terminal moiety on the surface hydrophilicity and cell interaction. The hPEGs were utilized in the hydrogel formulation following procedures described hereinabove. See also references (10-13). The network thus formed is a random copolymer of Ac, TMPTA, and hPEG, with, for example, an acrylate ω-terminal and an α-terminal of a different chemical moiety (XPEGmA).

Specifically, XPEGmAs were grafted to a gelatin polymer matrix with various dangling terminal functional groups and incorporated throughout the polymer matrix by copolymerizing the acrylate terminal into a randomly polymerized network of Ac and TMPTA.⁽¹⁰⁻¹³⁾ This type of polymer network containing M-PEG is nonionic, low swelling, glassy when dry, optically transparent, and colorless.⁽¹⁰⁻¹³⁾ In spite of the relatively high mass fraction of M-PEGs present, minimal swelling was observed for the polymer due to the highly cross-linked and hydrophobic nature of the TMPTA network. Differential scanning calorimetry analysis showed that these materials are completely amorphous and the M-PEG component is completely phase-mixed in the cross-linked TMPTA matrix.⁽¹⁰⁾

The surface hydrophilicity of XPEGmA-co-Ac-co-TMPTA networks was quantified with an underwater air bubble captive system. The hydrogel was completely suspended in water that was maintained at a physiologically-relevant temperature of 37.5° C. An air bubble was placed at the down side of the gel and the contact angle was measured using a modified computer-assisted video contact angle system (AST Inc). Measurement was made at six randomly selected areas, averaged, and repeated three times on three different polymer samples (n=3). Because the air bubble contact angle was measured through the aqueous phase and performed under water, the value obtained is essentially the water-receding contact angle; furthermore, the higher the contact angle, the higher is the hydrophilicity of the film.

The gels so formed were then evaluated for their interaction with cultured cells. Human neonatal dermal fibroblasts at a concentration of 75,000 per 1 ml of Fibroblast Basal Medium with human fibroblast growth factor-b (0.5 mg/ml), insulin (0.5 mg/ml), and 5% fetal bovine serum (Clonetics, San Diego, California) were incubated with the XPEGmA-co-Ac-co-TMPTA network. At 2, 24, and 48 hr thereafter, adherent cell morphology and density were manually quantified using a computer-assisted video analysis system coupled to an inverted light microscope.

All experimental results are expressed in mean±standard deviation (S.D.). Each sample was independently repeated three times (n=3). Comparative analyses were performed with Statview® 4.5 using analysis of variance and Fisher's protected least significant difference test at 95% confidence level (p<0.05).

¹³C-NMR chemical shifts for M-PEG, CN-PEG, COOH-PEG, and PT-PEG intermediates and final products synthesized from 2 kDa PEG precursors are listed in Table 1.

TABLE 1
¹³C-NMR chemical shifts (ppm) far M-PEG, CN-PEG, COOH-PEG, and PT-PEG
critical intermediates and final products synthesized from 2K Da PEG-diol precursors

| Chemical Shifts of Designated Carbon (In superscript) with | | | | | | | | | | | | |
|---|---|---|---|---|---|---|---|---|---|---|---|---|
| Compounds with the Following General Structure | | | | | | | | | | | | Chemical Group or |
| n-CH₂ | C^{α1} | C^{β1} | C^{α2} | C^{β2} | C⁽¹⁾ | C⁽²⁾ | C⁽³⁾ | C⁽⁴⁾ | C⁽⁵⁾ | C⁽⁶⁾ | C⁽⁷⁾ | Terminal Y |
| Y—C^{(α1)}H₂C^{(β1)}H₂O(CH₂CH₂O)ₙC^{(β2)}H₂C^{(α2)}H₂OH | | | | | | | | | | | | |
| 70.4 | 61.3 | 72.4 | 61.3 | 72.4 | — | — | — | — | — | — | — | —OH |
| 70.5 | 30.3 | 71.2 | 62.9 | 72.3 | — | — | — | — | — | — | — | —Br |
| 70.4 | 19.8 | 66.2 | 62.3 | 72.4 | 119.0 | — | — | — | — | — | — | —C⁽¹⁾N |
| 70.4 | 68.2 | 70.2 | 61.3 | 72.4 | 53.7 | 171.2 | — | — | — | — | — | —OC⁽¹⁾H₂C⁽²⁾OOH |
| 70.4 | 64.4 | 71.8 | 61.6 | 69.7 | 58.2 | — | — | — | — | — | — | —OC⁽¹⁾H₃ |
| 70.5 | 68.6 | 69.2 | 61.6 | 72.5 | 163.6 | 114.7 | 131.6 | 130.0 | 21.8 | — | — | 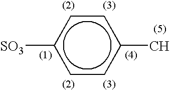 |
| Y—C^{(α1)}H₂C^{(β1)}H₂O(CH₂CH₂O)ₙC^{(β2)}H₂C^{(α2)}H₂OC⁽¹⁾OC⁽²⁾HC⁽³⁾H₂ | | | | | | | | | | | | |
| 70.5 | 64.6 | 71.1 | 63.9 | 68.2 | 165.3 | 130.6 | 128.2 | 58.2 | — | — | — | —OC⁽⁴⁾H₃ |
| | | | | | 169.5 | | | | | | | |
| 70.4 | 18.6 | 66.5 | 64.3 | 68.7 | 165.9 | 130.6 | 128.0 | 117.7 | — | — | — | —C⁽⁴⁾N |
| | | | | | 169.2 | | | | | | | |
| 70.5 | 68.5 | 70.8 | 64.6 | 68.4 | 166.0 | 130.9 | 128.2 | 53.6 | 170.3 | — | — | —OC⁽⁴⁾H₂C⁽⁵⁾OOH |
| | | | | | 169.5 | | | | | | | |
| 70.5 | 37.2 | 67.8 170.6 | 63.8 | 68.8 | 167.8 | 130.9 | 128.2 | 168.0 | 132.1 | 123.1 | 133.9 | 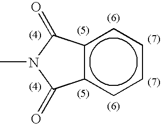 |

For all samples, the methyl stretch and the “b” carbon of the PEG chains were observed at approximately 68 to 72 ppm; whereas, the “a” carbon shift was highly dependent on the terminal group (Y). For compounds with a general structure of HOCH₂CH₂O(CH₂CH₂O)ₙCH₂CH₂—Y, where Y is —OH, —Br, —CN, —OCH₂COOH, —OCH₃, or tosyl group, the assigned carbon showed signals at the corresponding chemical shift. For the final acrylated product with a general chemical structure of X—CH₂CH₂O(CH₂CH₂O)ₙCH₂CH₂OCOCHCH₂, where X is: —OCH₃, —CN, —OCH₂COOH, or phthalimide, three unique chemical shifts were observed that correspond to the three carbons of the acrylate group. Specifically, the chemical shifts for —COO— stretch and —CHCH₂ stretch were observed at 165.3 to 170.6 and 128.0 to 130.9 ppm, respectively. In addition, appropriate chemical shifts were observed for the assigned carbon for each terminal group (Y). Similar NMR results were obtained when 5 kDa PEGs were utilized in lieu of 2 kDa PEGs as precursors in the synthesis scheme for all compounds shown in Table 1.

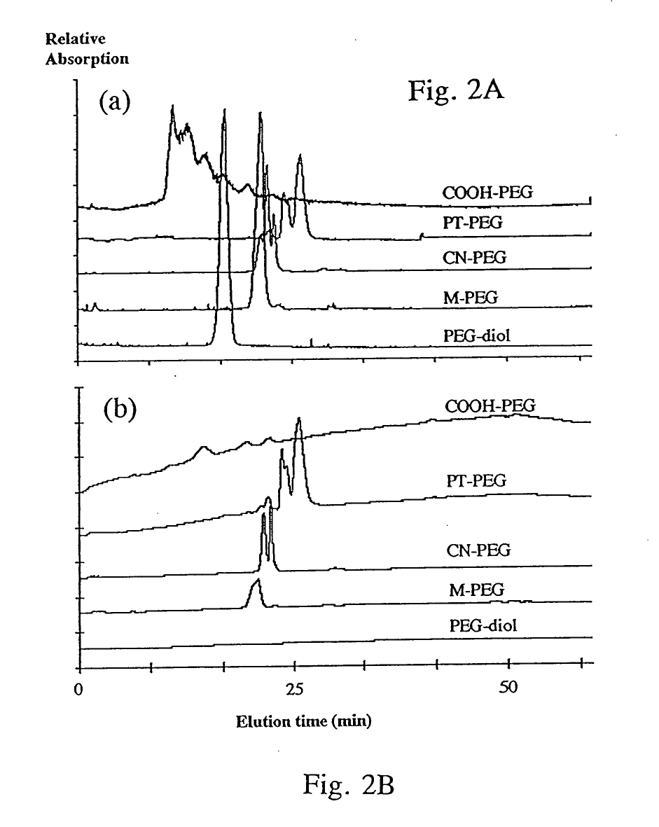

To determine percent conversions of the final product of M-PEG, CN-PEG, COOH-PEG, and PT-PEG, HPLC analyses performed (Table 2 and FIGS. 2A and 2B) from various HPLC detectors were utilized to elucidate the chemical structure of each individual peak of a given chromatogram. In addition, each fraction was collected with an automated fraction collector and re-analyzed using ¹H and ¹³C NMR to ascertain further the chemical composition(s) of each collected fraction. Results showed 100% conversion for M-PEG from the PEG starting material. CN-PEG showed approximately 65% conversion with no other acrylated side products. PT-PEG showed an approximate 65% conversion with about 5% of the final product containing another acrylate side-product (e.g., α-tosyl-ω-acrylate-PEG). COOH-PEG showed an approximate 60% conversion with an additional 10% of other acrylated side-products (e.g., α-hydroxyl-ω-acrylate-PEG and bis-acrylate-PEG).

These results validate the synthesis of the XPEGmAs that were employed as a main component in the hydrogel synthesis. Based on the gel synthesis scheme, hPEG containing one or more acrylate groups will be covalently incorporated into the network; whereas, those without any acrylate groups will be removed from the network after the equilibration step in water as a part of the network formation procedure. Although the final product of each XPEGmA was not further purified prior to the polymer synthesis, the low concentration of other acrylated side products plays a minimal role in the network composition.

TABLE 2
Comparison of HPLC retention time, normalized peak area, and percent
conversion for M-PEG, CN-PEG, COOH-PEG, and PT-PEG synthesized 2K Da PEG-diol
precursors

| PRC | Retention | Normalized | Conversion | UV | PEG Derivative |
|---|---|---|---|---|---|
| Product | Time (min) | Peak Area | Factor (%) | Signal | Identification |
| PEG-diol | 16 | 1.0 | 1 | no | α-methyl-ω-hydroxyl |
| M-PEG | 21 | 1.0 | 100 | strong | α-methyl-ω-acrylate |
| CN-PBG | 21 | 1.1 | 13 | strong | bis-ethylcyano |
| | 23 | 5.2 | 63 | strong | α-ethylcyano-ω-acrylate |
| | 24 | 2.0 | 24 | no | α-nitrile-ω-hydroxy |
| COOH-PEG | 11 | 2.5 | 14 | no | bix-carboxyl |
| | 13 | 2.3 | 13 | no | α-carboxyl-ω-hydroxyl |
| | 15 | 10.1 | 57 | weak | α-carboxyl-ω-acrylate |
| | 16 | 1 | 6 | no | bis-hydroxyl |
| | 19 | 1.2 | 7 | weak | α-hydroxyl-ω-acrylate |
| | 23 | 0.6 | 3 | weak | bis-acrylate |
| PT-PEG | 22 | 2.0 | 7 | weak | α-tosyl-ω-acrylate |
| | 24 | 8.1 | 26 | strong | bis-phthalimide |
| | 26 | 19.5 | 64 | strong | α-phthalimide-ω-acrylate |

These heterobifunctional intermediates and final products of XPEGmA are stable under storage in vacuo at room temperature and can be modified further by a broad range of chemical methods for various applications. For example, the phthalimide group is a good protecting group that can be hydrolyzed to form primary amines.

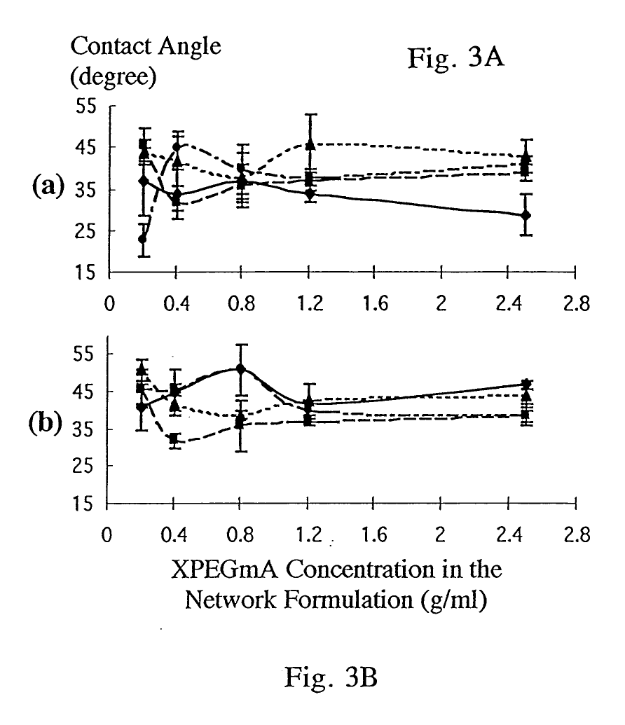

A previously developed polymer network formulation was adopted to elucidate the effect of the PEG chemistry on the surface characteristics of the resulting hydrogels. Polymer networks containing various XPEGmAs at several concentrations and different molecular weights were transparent or translucent. The network surface hydrophilicity was quantified using an under water contact angle system and was found to be dependent of three factors: the molecular weight of the starting material PEG, the dangling terminal functional group, and the concentration of the XPEGmA in the network (see Table 3 and FIGS. 3A and 3B).

TABLE 3
Surface hydrophilicity of the XPEGmA-co Ac-co TMPTA network
containing XPEGmA of various concentration, molecular weight,
and terminal moiety

| | XPEGmA concentration in | | | | |
|---|---|---|---|---|---|
| XPEGmA | the network formulation (g/ml) | | | | |
| type | 0.2 | 0.4 | 0.8 | 1.25 | 2.5 |
| 2K (Da) | | | | | |
| M-PEG | 37 ± 8 | 34 ± 6 | 37 ± 4 | 34 ± 2 | 29 ± 5 |
| CN-PEG | 46 ± 4 | 32 ± 2 [symbol] | 36 ± 5† | 37 ± 2† | 39 ± 2†§ |
| COOH-PEG | 44 ± 3 | 42 ± 6 | 38 ± 6† | 46 ± 7§ | 43 ± 4§ |
| PT-PEG | 23 ± 4§ | 45 ± 4†§ | 40 ± 6† | 38 ± 2† | 41 ± 2†§ |
| 5K (Da) | | | | | |
| M-PEG | 41 ± 6 | 45 ± 6‡ | 51 ± 7‡ | 42 ± 5‡ | 47 ± 1‡ |
| CN-PEG | 46 ± 5 | 32 ± 2†§ | 36 ± 7 [symbol] § | 37 ± 1†§ | 39 ± 3‡§ |
| COOH-PEG | 51 ± 3 [symbol] § | 42 ± 2† | 39 ± 1†§ | 43 ± 4† | 44 ± 4 [symbol] |
| PT-PEG | 46 ± 4 [symbol] | 46 ± 1 | 51 ± 7 | 40 ± 3† | 39 ± 2 [symbol] § |

First, when a given concentration of XPEGmA containing a given dangling terminal group in the network was considered, an increase in the molecular weight of the terminal group significantly lowered the hydrophilicity of networks containing M-PEG (0.4 to 2.5 g/ml), COOH-PEG (0.2 g/ml), or PT-PEG (0.2 g/ml). For other networks, the molecular weight of XPEGmA did not significantly affect the hydrophilicity.

Second, the different terminal moiety of XPEGmA showed a variable effect on the surface hydrophilicity when compared with that of M-PEG of given molecular weight and concentration.

Third, the XPEGmA concentration in the network formulation showed various correlations with hydrophilicity. Because XPEGmA was employed in the network formation without further purification, the potential effect of differential percent conversion of acrylated hPEG on surface hydrophilicity must be addressed. M-PEG showed a 100% conversion and the network containing M-PEG demonstrated no changes in surface hydrophilicity with increasing M-PEG concentration. Whereas for other XPEGmAs, various correlations among hydrophilicity and the type, percent conversion (ca. 60 to 100%), and concentration were observed. Hence, it was concluded that the percent conversion of XPEGmA within 60 to 100% did not affect the dependency of XPEGmA concentrations on hydrophilicity. These analyses determined that the network surface hydrophilicity was predominately influenced by the XPEGmA concentration in the network formulation with the molecular weight and the terminal moiety playing lesser roles.

Next, XPEGmA-co-Ac-co-TMPTA networks containing various XPEGmAs at several concentrations were employed to determine the effect of surface characteristics of the gel on human fibroblast adhesion. All adherent cells showed extensive pseudopodial extension and cytoplasmic spreading, with some cells exhibiting polar cell body morphology. The results (see Table 4) showed that adherent cell density was primarily dependent on the XPEGmA concentration in the network formulation. Specifically, adherent cell density decreased with increasing XPEGmA concentration at all culture time. No adherent cell was observed on networks containing XPEGmA concentration between 1.25 to 2.5 g/ml at all culture times. These trends were independent of the XPEGmA molecular weight and terminal moiety. No direct mechanistic correlation can be made between network surface hydrophilicity and adherent cell density because several interrelated complex parameters (e.g., XPEGmA chemicophysical properties, adsorption of serum adhesion-mediating proteins, etc.) contribute to these two phenomena. However, the adherent cell density decreased with increasing XPEGmA concentration for all samples.

TABLE 4
Adherent human dermal fibroblast density on the XPEGmA-co-Ac-co-TMPTA
network containing XPEGmA of various concentration, molecular weight, and terminal moiety

| | Culture Time (hr) | | | | | | | | | | | | | | |
|---|---|---|---|---|---|---|---|---|---|---|---|---|---|---|---|
| | 2 hr | | | | | 24 hr | | | | | 48 hr | | | | |
| XPEGmA | XPEGmA concentration in the network formulation (g/ml) | | | | | | | | | | | | | | |
| type | 0.2 | 0.4 | 0.8 | 1.25 | 2.5 | 0.2 | 0.4 | 0.8 | 1.25 | 2.5 | 0.2 | 0.4 | 0.8 | 1.25 | 2.5 |
| 2K (Da) | | | | | | | | | | | | | | | |
| M-PEG | 3 ± 2 | 3 ± 2 | 0 | 0 | 0 | 5 ± 2 | 2 ± 1 | 0 | 0 | 0 | 3 ± 2 | 4 ± 2 | 1 ± 1 | 0 | 0 |
| CN-PEG | 5 ± 4 | 2 ± 1 | 1 ± 1 | 0 | 0 | 3 ± 2 | 4 ± 3 | 1 ± 1 | 1 ± 1 | 0 | 5 ± 34 | 4 ± 3 | 3 ± 1 | 5 ± 2 | 0 |
| COOH-PEG | 2 ± 1 | 1 ± 0 | 1 ± 1 | 0 | 0 | 3 ± 1 | 2 ± 1 | 6 ± 4 | 0 ± 0 | 0 | 3 ± 2 | 1 ± 1 | 1 ± 1 | 1 ± 1 | 0 |
| PT-PEG | 2 ± 1 | 2 ± 1 | 1 ± 1 | 1 ± 0 | 0 | 1 ± 0 | 3 ± 2 | 3 ± 2 | 0 | 0 | 3 ± 1 | 2 ± 1 | 3 ± 3 | 0 ± 0 | 0 |
| 5K (Da) | | | | | | | | | | | | | | | |
| M-PEG | 3 ± 1 | 3 ± 2 | 0 | 0 | 0 | 3 ± 3 | 3 ± 2 | 1 ± 1 | 0 | 0 | 2 ± 1 | 3 ± 2 | 1 ± 1 | 0 | 0 |
| CN-PEG | 2 ± 2 | 1 ± 1 | 1 ± 0 | 0 | 0 | 4 ± 3 | 5 ± 4 | 0 ± 0 | 2 ± 1 | 0 | 3 ± 2 | 3 ± 2 | 0 ± 0 | 2 ± 2 | 0 |
| PT-PEG | 3 ± 1 | 1 ± 0 | 0 ± 0 | 0 | 0 | 2 ± 1 | 2 ± 2 | 0 ± 0 | 0 | 0 | 3 ± 1 | 3 ± 2 | 1 ± 1 | 0 | 0 |
| COOH-PEG | 4 ± 0 | 1 ± 1 | 1 ± 0 | 0 | 0 | 4 ± 2 | 2 ± 1 | 1 ± 0 | 1 ± 0 | 0 | 2 ± 1 | 3 ± 1 | 1 ± 0 | 0 ± 0 | 0 |

All values are expressed in × 100 cells/mm² (rounded-off for clarity, mean ± S.D., n = 3).

The results of this Example show that the presence of two distinct chemical moieties (i.e., carboxylic acids of the poly-acrylic acid backbone and the distinct functional group at the dangling terminus of XPEGmA grafted at the pendent chain configuration) within the hydrogels can be employed to bind (covalently) two or more distinct types of biofunctional molecules such as peptides and pharmaceutics by employing distinct chemical methodologies. Furthermore, the high content of PEGs in this system reduced protein adsorption and effectively eliminated nonspecific cell adhesion that would occur as a result, thus permitting the modulating of cellular function mediated uniquely by the multiple immobilized biofunctional agent (10-13). The invention thus provides multi-functional hydrogels that can be used, for example, to study complex biological systems and to deliver therapeutic agents locally and systemically.

### Example 2

### Drug Release Kinetics

This Example explores the swelling and drug release kinetics of various gelatin-based hydrogels. The hydrogels were cross-linked by various means, and contained various modifications of the gelatin backbone. The effect of pH on the drug release kinetics of these gels was also investigated.

As noted above, cross-linking gelatin produces a hydrogel of high molecular weight and reduces or prevents gelatin dissolution. The cross-linking agents used in this Example were: 0. 1%, 0.01%, and 0.001% (v/v) glutaraldehyde aqueous solutions, and self-cross-linking via liquid nitrogen immersion followed by baking. The backbone modifications to the gelatin were the addition of polyethylene glycol (PEG) or ethylenediaminetetraacetic dianhydride (EDTAD) or both. PEG has low immunogenicity and cytotoxicity. EDTAD has low toxicity and the lysyl residues of gelatin can be modified with EDTAD in a relatively fast reaction following facile procedures. See Hwang & Damodaran (1996) J. Agric. Food. Chem. 44:751-758. Also, modifying gelatin with EDTAD introduces polyanionic molecules into the gelatin chain, thereby improving the swelling capability of the gelatin hydrogels. The pHs investigated in this Example were pH 4.5, pH 7.0 and pH 7.4. Based on the swelling/degradation and drug release kinetics of these hydrogels under the stated conditions and in vivo analysis, these hydrogels are suitable as support matrices for the regeneration of rat neutral stem cells and as a drug carrier in mediating inflammation in vivo.

PEG diol (Aldrich, Mₙ 2 kD) was converted to PEG dialdehyde (PEGdial) by reacting PEG with acetic anhydride in DMSO in a molar ratio of 1:80:140 for 4 hours at 25° C. The composition of PEG dialdehyde was confirmed using the reverse-phase HPLC system and parameters as described in Example 1. This reaction produces a mixed product of PEG monoaldehyde and PEG dialdehyde. PEG dialdehyde had an elution time of approximately 11.5 min. and was approximately 80 wt % of the final product.

The lysyl amino groups of gelatin samples (Sigma, St. Louis, Mo.; Type A, from porcine skin, 300 bloom, cell culture tested) were modified by PEGdial to form PEG-modified gelatin (PG). Gelatin samples were also modified using EDTAD (Aldrich) to form EDTAD-modified gelatin (EG). Still further gelatin samples were modified with PEGdial and EDTAD to yield PEG-modified-EDTAD-modified gelatin (P/EG). PG or P/EG was created by adding PEGdial dissolved in 10 ml of H₂O (Milli-Q synthesis, 18.2 MΩ-cm, Millipore) and NaCNBH₃ dissolved in 10 ml of H₂O separately and simultaneously to a 5% (w.v) gelatin or EG solution at 50 to 60° C. for 24 hours in a wt ratio of gelatin/EG: PEGdial: NaCNBH₃ of (1:0.66:0.186). The theoretical maximum percent modification using this method is 100% modification of gelatin lysyl residues, based on an average 300 bloom gelatin molecular weight and average lysine content of the gelatin. See, e.g., Merck Index, 12^{th} Ed. (1996) #4388, p. 742. EG was created by adding EDTAD to a 1% (w/v) gelatin solution at pH 10, 40° C. for 3 hours in a wt ratio of gelatin:EDTAD of 1:0.034. The theoretical maximum percent modification of gelatin lysyl residues using this method is 38%. Thus, modifications larger than this indicate that both functional groups of the added EDTAD have bonded to lysyl residues in the gelatin, thereby cross-linking the gelatin chains. The level of gelatin modification was quantified using the 2,4,6-trinitrobenzene sulfonic acid spectrophotometric method. See Hwang & Damodaran, supra, and Offner & Bubnis (1996) Pharm. Res. 13:1821-1827.

To make the hydrogels, 10% (w/v in H₂O) solutions of gelatin (G), 10% PG, 40% EG and 60% P/EG were heated to approximately 70° C. and poured into petri dishes (60×15 mm, Cole-Parmer) to a thickness of 6 mm and allowed to set at room temperature overnight. Hydrogels were cut into 1 cm diameter circular discs or into 0.5×0.5 cm squares, and cross-linked with 0.1, 0.01 or 0.001% (v/v in H₂O) gluteraldehyde (Electron Microscopy Sciences, EM grade, 10% (v/v) aqueous solution) for 6 hours with gentle shaking. Cross-linked hydrogels were washed with H₂O ten times for 3-5 min. Washed hydrogels were left overnight in H₂O for continued leaching of the gluteraldehyde. Hydrogels were then dried at room temperature in ambient air for 48 hours and weighed. Separately, hydrogels of 10% by wt gelatin were dried in ambient air for 48 hours, frozen in liquid nitrogen for 30 seconds to 1 minute and then baked at 130-135° C. for 8.5 hours (self-cross linked; LN₂-baked G). Not all hydrogel formulations withstood the cross-linking, washing and drying steps, mainly due to hydrolysis. The hydrogel formulations that were included in the swelling/degradation and in vitro drug release studies were the 0.1% glutaraldehyde cross-linked G, PG, EG, and P/EG gels; the 0.01% glutaraldehyde cross-linked G, PG, and EG gels; the 0.001% glutaraldehyde cross-linked G and PG gels; and the self-cross linked LN₂-baked G. Swelling study results for P/EG hydrogels and in vitro and in vivo drug release studies are ongoing and results are not included here.

For in vitro drug release studies, each hydrogel was loaded with chlorhexidine digluconate (CHD; Sigma, 20% (w/v) aqueous solution) using the same drug loading density used for dexamethasone in the in vivo studies (150 μg/kg/day, dosage of 21 d). Assuming a rat weight of 0.2 kg, this loading density is equivalent to 630 μg/hydrogel. Based on the maximum swelling weight ratios from the swelling studies, each hydrogel was loaded with 35 μL of CHD (18 mg/ml), a volume well below the maximum volume the hydrogel could absorb. Hydrogels (0.5×0.5×0.6 cm) were placed into individual wells in a 48-well tissue culture plate. CHD was added to each well, and the hydrogels were allowed to absorb the drug solution overnight (approximately 15 hours) with gentle shaking.

To evaluate swelling and degradation kinetics, dried hydrogels were placed in 5 ml of aqueous solutions of pH 4.5, pH 7.0 or pH 7.4 in a water bath at 37° C. Aqueous solutions were created by adjusting the pH of H₂O with dilute HCl and NaOH. Hydrogels were transferred to fresh aqueous solutions at approximately 3 and 6 wks. Swollen hydrogels were weighed at 2, 4, and 6 hours, 1, 2, 3, 4, and 5 days, and 1, 2, 3, 4, 5, 6, 7, and 8 weeks to characterize the swelling/degradation kinetics. Extreme care was taken to preserve the integrity of the hydrogels at every step in the weighing process. The swelling weight ratio at each time point for each hydrogel was calculated as: (Wₛ−W_{d})/W_{d}, where Wₛ is the weight of the swollen gel and W_{d} is the weight of the dry gel (in grams). The maximum swelling weight ratio that occurred over 8 weeks and the time it occurred was also calculated (Rₘₐₓ & Tₘₐₓ, respectively). The last attainable swelling weight ratio (due to hydrogel dissolution) and the time it occurred was also calculated (R_{fail} & T_{fail}, respectively). Statistical analysis was performed using ANOVA and Tukey multiple comparisons tests (p<0.05). Individual sample solutions from the swelling study were collected for ongoing GPC analysis of degradation products (results not shown) (20% (v/v) acetonitrile: 0.1 M NaNO₃ at a flow rate of 0.7 ml/min, 60 min., using three Ultrahydrogel colunms in series, Ultrahydrogel 250, 1000 and Linear, on a Waters system).

For in vivo studies, unmodified gelatin cross-linked in 0.1% and 0.01% gluteraldehyde were tested in vivo, following the established cage implant system. See Kao & Anderson (1999) “Handbook of Biomaterials Evaluation 2^{nd} ed., Taylor & Frances Publishing, Philadelphia, Pa., pp. 659-671. Samples were placed inside a cylindrical cage (3.5 cm long×1 cm diameter) constructed from medical grade stainless steel wire mesh. Empty cages were implanted as controls. All cages were implanted subcutaneously in the back of 3-month-old female Sprague-Dawley rats. At 4, 7, 14 and 21 days post-implantation, the inflammatory exudates that collected in the cages were withdrawn and analyzed for the quantitative evaluation of cellular and humoral response to implantation using standard hematology techniques. The distributions of lymphocyte, monocyte, and polymorphonuclear leukocyte (PMN) subpopulations in the exudates were determined. Concurrently, the implanted materials were retrieved for analysis of changes in the sample physiochemical composition (e.g., percent mass loss).

Percent modification of the lysyl residues in gelatin by PEG and/or EDTAD was quantified using the TNBS method: The PG was found to be 10% modified, the EG 40% modified, and the P/EG 60% modified. All results reported here incorporate materials from the same batch of modified gelatin (i.e. 8% PG, 42% EG).

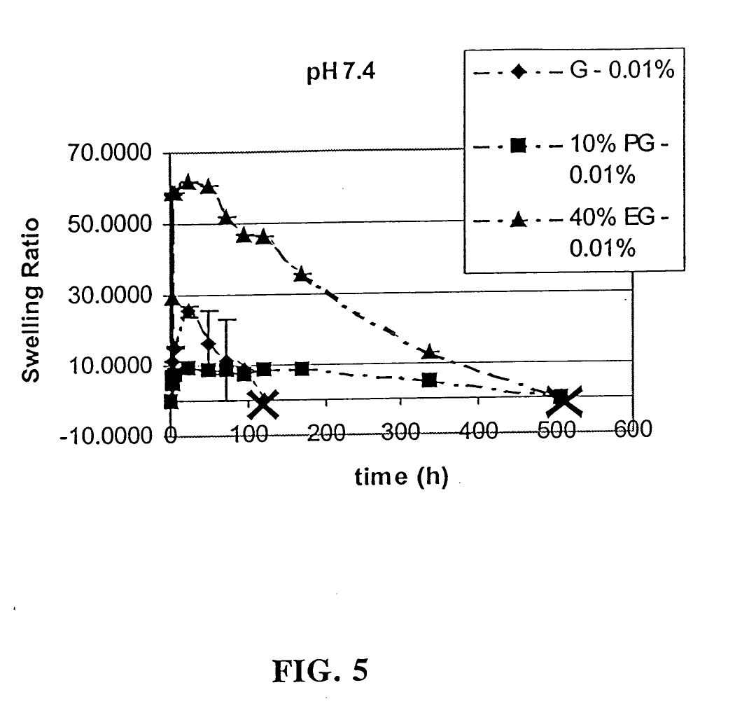

FIG. 5 is a graph depicting representative swelling/degradation kinetics. Time in hours is shown on the X-axis; swelling ratio is shown on the Y-axis. Key: G, 0.01% glutaraldehyde cross-linked=♦; 10% PG, 0.01% glutaraldehyde cross-linked=▪; 40% EG, 0.01% glutaraldehyde cross-linked=▴. Swelling/degradation studies showed that G modified with PEG significantly increased Tₘₐₓ and T_{fail}, whereas G modified with EDTAD significantly increased Tₘₐₓ. Hydrogels cross-linked in 0.01% or 0.001% gluteraldehyde showed a significant difference in Tₘₐₓ and T_{fail} over gels cross-linked in 0. 1% gluteraldehyde. The level of pH did not significantly affect Rₘₐₓ, Tₘₐₓ, R_{fail} and T_{fail}. Table 5 shows Rₘₐₓ, Tₘₐₓ, R_{fail} and T_{fail} for all levels of gluteraldehyde concentration, pH and gelatin backbone modification.

TABLE 5
R_{MAX}, T_{MAX}, R_{FAIL}, AND T_{FAIL} FOR ALL LEVELS OF
GLUTERALDEHYDE/HEAT TREATMENT, PH AND GELATIN
BACKBONE MODIFICATION

| % | | | | | | |
|---|---|---|---|---|---|---|
| gluteraldehyde | | | | | | |
| fixation/heat | | G | | | | |
| treatment | pH | Mod^{c} | R-max | T-max | R-fail | T-fail |
| 0.1% | 4.5 | G | 6.30 | 108 | 4.11 | >1344 |
| | | PG | 6.98 | 1344^{b} | 6.98 | >1344 |
| | | EG | 8.77 | 720 | 7.71 | >1344 |
| | 7.0 | G | 5.94 | 108 | 2.88 | >1344 |
| | | PG | 6.64 | 1092 | 4.55 | >1344 |
| | | EG | 12.04 | 1008 | 6.24 | >1344 |
| | 7.4 | G | 4.68 | 96 | 1.45 | 1092 |
| | | PG | 6.60 | 1092 | 5.35 | >1344 |
| | | EG | 894.17 | 924 | 892.52 | >1344 |
| 0.01% | 4.5 | G | 35.48 | 36 | 7.25 | 132 |
| | | PG | 11.54^{b} | 24 | 5.80 | 420 |
| | | EG | 31.53 | 2 | 14.07 | 84 |
| | 7.0 | G | 40.23 | 48 | 8.49 | 84 |
| | | PG | 10.63 | 96 | 8.36 | 336 |
| | | EG | 26.96 | 2 | 7.71 | 168 |
| | 7.4 | G | 26.29 | 36 | 8.21 | 72 |
| | | PG | 10.48 | 96 | 5.06 | 336 |
| | | EG | 30.88 | 12 | 6.47 | 168 |
| 0.001% | 4.5 | G | 0.10 | 1 | −0.01 | 2 |
| | | PG | 0 | 0 | 0 | 0 |
| | | EG | — | — | — | — |
| | 7.0 | G | 0.33 | 1 | 0.17 | 2 |
| | | PG | 0 | 0 | 0 | 0 |
| | | EG | — | — | — | — |
| | 7.4 | G | 0.36 | 1 | 0.36 | 1 |
| | | PG | 0 | 0 | 0 | 0 |
| | | EG | — | — | — | — |
| LN₂-baked G | 4.5 | G | 3.96 | 24 | 2.06 | 2.52^{b} |
| | 7.0 | G | 4.76 | 24 | 0.40 | 72 |
| | 7.4 | G | 4.05 | 15 | 72 | 96 |

^{a}All values expressed in mean (n = 2-3) with s.e.m. omitted for clarity.
^{b}significantly different from G under same experimental conditions; paired t-tests, p < 0.05.
^{c}10% PG or 40% EG

In vivo studies following the cage implant system allowed the duration and magnitude of the host foreign body reaction to the implanted gelatin-based hydrogels (0.1% G and 0.01% G) to be evaluated. The presence of a high concentration (relative to control) of polymorphonuclear leukocytes (PMNs) in the exudates indicates an acute inflammatory response, which occurs at the onset of implantation and attenuates with time. The presence of a high concentration (relative in control) of monocytes and lymphocytes in the exudates is indicative of the chronic inflammatory response. Thus, 0.1% G hydrogels elicited a slightly enhanced chronic inflammatory response at 7 days and an enhanced chronic inflammatory response at 14 days vs. the control and that of 0.01% G. 0.01% G elicited a slightly enhanced chronic inflammatory response at 7 days vs. the control (see Table 6). By day 21, all samples showed a comparable level of chronic inflammation vs. the controls the proceeded toward resolution. Percent mass loss of samples increased with increasing implantation time and was further increased with decreasing percentage of gluteraldehyde fixation (results not shown).

TABLE 6
TOTAL AND DIFFERENTIAL LEUCOCYTE CONCENTRATION IN
THE INFLAMMATORY EXUDATES OF GELATIN HYDROGELS
CROSS-LINKED IN 0.1 OR 0.01% GLUTERALDEHYDE

| | |
|---|---|
| Implan- | |
| tation | Cell concentration (×cells/μL)^{a} |

| Sample | time (day) | Total | Lymphocyte | Monocyte | PMN |
|---|---|---|---|---|---|
| Empty | 4 | 184 ± 25 | 168 ± 23 | 16 ± 7 | 1 ± 1 |
| cage | 7 | 57 ± 12^{c} | 49 ± 10^{c} | 7 ± 2 | 0 ± 0 |
| (no | 14 | 55 ± 7 | 36 ± 3 | 12 ± 4 | 7 ± 5 |
| sample) | 21 | 91 ± 69 | 98 ± 54 | 20 ± 16 | 0 ± 0 |
| 0.1% | 4 | 597 ± 392 | 255 ± 116 | 126 ± 113 | 217 ± 212 |
| | 7 | 183 ± 129 | 78 ± 40 | 26 ± 14 | 79 ± 74^{b} |
| | 14 | 235 ± 65^{b} | 118 ± 30^{b} | 40 ± 16 | 77 ± 75 |
| | 21 | 200 | 167 | 33 | 0 |
| 0.01% | 4 | 477 ± 195 | 412 ± 172 | 57 ± 28 | 8 ± 5 |
| | 7 | 178 ± 78^{b} | 157 ± 80 | 17 ± 1^{b} | 4 ± 3 |
| | 14 | 72 ± 36 | 60 ± 29 | 10 ± 7 | 2 ± 1 |
| | 21 | 93 ± 3 | 72 ± 5 | 9 ± 4 | 12 ± 11 |

^{a}All values expressed in mean ± s.e.m. (n = 3-7).
^{b}Represents p < 0.01 vs. respective values of “empty cage” controls.
^{c}Represents p < 0.01 vs. respective values at day 4 of the same sample type.

This Example shows that gelatin backbone modifications and cross-linking agent selection affect the swelling/degradative kinetics of modified gelatin-based hyrdogels. By modulating these material properties and monitoring how these changes affect drug release kinetics, a nonimmunogenic, bioresorbable cell/drug carrier matrix can be made that will have desirable release characteristics based on such consideraions as the drug being used in the formulation, the length of the treatment, and the condition being treated, and the location of the implanted matrix.

### Example 3

### In Vivo Modulation of Host Response Using Gels Grafted with Fibronectin-Derived Biomimetic Oligopeptides

The host inflammatory reaction is a normal response to injury and the presence of foreign objects. The magnitude and duration of the inflammatory process have a direct impact on biomaterial biostability and biocompatability. Thus, this Example investigates the performance of gels fabricated according to the present invention that include fibronectin-derived biomimetic oligopeptides. Fibronectin in known to adsorb on a variety of biomaterials and play an important role in the host-foreign body reaction. The RGD (SEQ. ID. NO: 1) and PHSRN (SEQ. ID. NO: 2) amino acid sequences are particularly interesting because these sequences are present on adjacent loops of two connecting FIII modules and bind synergistically to a host of integrins.

Oligopeptides were designed based on the primary and tertiary structure of human plasma fibronectin to study the structure-functional relationship of RGD and PHSRN regions of fibronectin in regulating the host inflammatory response and macrophage behavior in vivo. Peptides included RGD and PHSRN sequences alone or in combination. The tertiary structure of fibronectin was utilized as a guide in the formulation of peptides. The distance between the PHSRN sequence and the RGD sequence within the natural fibronectin molecule in solution was approximated using the structural coordinates archived in the SwissProt Database® (sequence FINC_HUMAN P02751). Based on the measurement, a hexamer of glycine (G₆) of approximately the same length was used to link the two bioactive sequences in both possible orientations. A terminal trimeric glycine domain (G₃) was employed as a spacer in all peptides. Oligopeptides were synthesized using solid-resin methods on an automated peptide synthesizer (Millipore) using conventional 9-fluorenylmethyloxycarbonyl chemistry without further purification and with a final coupling efficiency of approximately≧85% purity. Peptides were characterized and analyzed using mass spectroscopy and reverse phase HPLC coupled to photodiode array, evaporative light scatter, and UV/Vis detectors. The following oligopeptides were synthesized: G₃RGDG (SEQ. ID. NO: 3), G₃PHSRNG (SEQ. ID. NO: 4), G₃RGDG₆PHSRNG (SEQ. ID. NO: 5), G₃PHSRNG₆RGDG (SEQ. ID. NO: 6), and G₃RDGG (SEQ. ID. NO: 7) as a nonspecific control. Peptides were covalently grafted onto hydrogels as described in Example 1 to investigate the influence of peptides on the host response and macrophage behavior in vivo.

The gels used in this Example were random co-polymers of monomethoxy polyethyeneglycol monoacrylate (mPEGmA), acrylic acid (Ac), trimethylolpropane triacrylate (TMPTA). As noted above, these gels are hydrophilic, nonionic, low swelling, glassy, optically transparent, and colorless. Differential scanning calorimetry analysis showed that these materials are completely amorphous and the mPEGmA component is completely phase-mixed in the cross-linked TMPTA matrix. The bioactive oligopeptides were grafted onto mPEGmA-co-Ac-co-TMPTA hydrogels and the resulting gels mediated cell adhesion in a receptor-peptide specific manner. The peptide surface density was found to be dependent on the number of amino acids per peptide. For example pentapeptides were grafted at 66±6 pmol/cm² surface density; whereas, peptides containing 30 residues were grafted at approximately one-fifth of that surface density. In this Example, oligopeptides containing one bioreactive region (i.e., G₃RGDG, G3PHSRNG, and G₃RDGG) were grafted at about twice the density of oligopeptides containing two bioreactive regions (i.e., G₃RGDG₆PHSRNG and G₃PHSRNG₆RGDG).

The well-established subcutaneous cage-implant system was utilized to study the effect of implanted materials on the host foreign body reaction. Briefly, mPEGmA-co-Ac-co-TMPTA networks grafted with or without fibronectin-derived peptides were placed in sterile water for at least 48 hours to remove low molecular weight leachable residual molecules from the polymerization process and to achieve hydration equilibrium. The polymer samples were then inserted under sterile conditions into an autoclaved cylindrical cage measured 3.5 cm long, 1 cm in diameter, and constructed from medical grade stainless steel wire mesh. Cages containing various polymer samples were subcutaneously implanted at the back of 3-month old female Sprague-Dawley rats. Empty cages were employed and implanted as controls. The inflammatory exudate that collects in the cage was withdrawn at 4, 7, 10, 14, and 21 days post-implantation and analyzed for the quantitative evaluation of cellular and humoral response to the test material using standard and conventional hematology techniques. Specifically, the distribution of lymphocyte, monocyte, and PMN subpopulations in the exudate was determined. The presence of a high concentration of PMNs in the inflammatory exudate indicates an acute inflammatory response, which occurs from the onset of implantation and attenuates with time. This is followed by the chronic inflammatory response, which is characterized by the presence of a high concentration of monocytes and lymphocytes in the exudate. Hence, the cage implant system allows the host inflammatory reaction to the test sample to be observed as a function of time and material property. A drop of each exudate sample was also cultured on brain-heart infusion agar plates to check for incidence of infection. No infection was observed at any retrieval time for any sample. At 4, 7, 14, 21, 35, and 70 days post-implantation, test polymer samples were retrieved and the adherent cell morphology and density were quantified using a video analysis system coupled to a light microscope.

A previously developed mathematical model describing the in vivo kinetics of macrophage fusion on various biomaterials was employed to provide insights into the effect of materials and peptides on foreign body giant cell (FBGC) formation. The model was formulated based on Flory's most-probable molecular weight distribution of polymer chains. In the analysis, each adherent macrophage is analogous to a monomer and the process of cell fusion is analogous to the polymerization process. Two initial premises are necessary: (1) the FBGC size is directly proportional to the number of nuclei in a given FBGC; and (2) the ability for each cell to fuse is constant and independent of the cell size. The FBGC size-distribution equation Nₓ=p^{ax-3}(1−p) was applied to the measured FBGC size-distribution result of each sample at each retrieval time. N, is the cell size number-fraction of FBGCs with area x; p is the probability of cell fusion or the ratio of the number of cell fusion to the initial adherent macrophage density; a is a constant relating to the number of nuclei per FBGC to the cell area (FBGC/mm²) and has been found to be constant for various clinically relevant biomaterials under different mechanical stress conditions. See Kao et al. (1994) J. Biomed. Mater. Res. 28:73-79; Kao et al. (1995) J. Biomed. Mater. Res. 29(10);1267-75; and Kao et al. (1994) J. Biomed. Mater. Res. 2:819:829. Values for p and a were obtained through a curve-fit iteration until r²>0.98. The resulting values of p for each sample at each retrieval time were utilized to calculate two kinetic parameters that characterize the process of cell fusion: the density of adherent macrophages that participate in the FBGC formation (d₀=d_{f}/[2(1−p)] and the rate constant of cell fusion (1/(1−p)=d₀tk+1), where d₀ is the calculated density of adherent macrophages that participate in the FBGC formation process (macrophages/mm²), d_{f} the measured adherent macrophage density at 4 days post-implantation (macrophages ¹mm⁻²), t the implantation time (week), and k the inverse rate constant of cell fusion (mm²cell⁻¹ week⁻¹).

All experimental results are expressed in mean±standard error of the mean. Each sample was independently repeated 3 times (n=3). Comparative analyses were performed with Statview® 4.5 using analysis of variance and Fisher's protective t-test at 95% confidence level (p<0.05).

Total and differential leukocyte analysis was performed at several post-implantation periods (Table 7). No PMNs were observed at any time point for all samples, indicating that the presence of empty cages and networks grafted with or without fibronectin-derived biomimetic oligopeptides elicited a rapid acute inflammatory response that was resolved within 4 days of implantation. For the empty cage control, total leukocyte and lymphocyte concentrations decreased rapidly between 4 and 7 days post-implantation and remained steady thereafter up to 21 days. Monocyte concentration remained constant from 4 to 21 days post-implantation. These results indicate that the presence of the empty cage elicited a rapidly decreasing chronic inflammatory response by 7 days post-implantation that turned toward resolution with increasing implantation time. The presence of mPEGmA-co-Ac-co-TMPTA gels within the cage showed a constant total leukocyte concentration from 4 to 21 days of implantation. However, the presence of the gels increased monocyte concentration and lowered lymphocyte concentration at days 4 and 7 when compared with that of empty cage controls, suggesting a comparable level of chronic inflammatory response that turned toward resolution but with an altered leukocyte sob-population distribution. When comparing the trends between mPEGmA-co-AC-co-TMPTA networks and empty cage controls, the presence of immobilized peptides on the polymer network did not significantly affect the total and differential leukocyte concentrations up to 14 days post-implantation, except that the decreased lymphocyte concentration was not observed for G₃RGDG-grafted networks at days 4 and 7 and for other peptide-grafted surfaces at day 7 of implantation.

These results indicate that the presence of polymer networks with or without immobilized peptides did not significantly modify the host acute and chronic inflammatory reactions up to 14 days of implantation. By 21 days of implantation, the presence of grafted G₃RGDG or G₃RDGG slightly decreased the total and lymphocyte concentrations when compared with respective values of “no grafted peptides” and “empty cage” controls. This trend was not observed for surfaces grafted with G₃PHSRNG₆RGDG. Conversely, the presence of grafted G₃PHSRNG or G₃RGDG₆PHSRNG slightly increased the total and lymphocyte concentrations when compared with respective values of “no grafted peptides” controls (p<0.05). At 21 days post-implantation and thereafter, extensive fibrous encapsulation at the exterior of all implanted cages and the absence of the inflammatory exudate inside the cage were observed for all samples, indicating the progression of tissue healing. These data suggest that the identity of grafted peptides did not significantly alter the temporal variation and intensity of the host acute and chronic inflammatory reaction.

Adherent macrophage density on implanted mPEGmA-co-AC-co-TMPTA networks grafted with or without fibronectin-derived oligopeptides was quantified at different retrieval times. In general, adherent macrophages on all surfaces decreased with increasing implantation time (see Table 8). Adherent macrophage densities for all samples were comparable and were higher than respective values of G₃RDGG or “no grafted peptide” controls at each retrieval time up to 14 days post-implantation. Adherent macrophage density on all samples was comparable from 21 to 70 days post-implantation. Adherent macrophages on all surfaces showed an extensive spread morphology with pseudopodial extension. These results indicate that peptides containing RGD and/or PHSRN motifs do not affect adherent macrophage density.

At each retrieval time up to 70 days post-implantation, no surface cracking, pitting, nor other evidence of physical degradation were observed under polarized light microscope at 40× magnification on any polymer sample with or without grafted peptides.

The morphology of FBGCs on all samples was that of foreign-body type, i.e., random arrangements of nuclei numbered more than three nuclei per cell with widely variable, extensive cytoplasmic forms. In general, FBGC density increased with increasing implantation time for all samples except that on surfaces grafted with G₃RGDG or G₃PHSRNG₆RGDG at which the adherent FBGC density remained constant with increasing implantation time (data not shown). In addition, the average FBGC size increased with increasing implantation time for all samples (data not shown).

These results showed that hydrogels grafted with fibronectin-derived peptides mediated extensive FBGC coverage that increased with increasing implantation time. Specifically, surfaces grafted with G₃RGDG₆PHSRNG showed the highest FBGC coverage at about 90% of the total sample area when compared with other sample types and controls at 70 days post-implantation. These in vivo findings indicate that the RGD motif, specifically in the configuration of G₃RGDG or G₃PHSRNG₆RGDG, but not G3RGDG₆PHSRNG, modulates a rapid macrophage fusion to form FBGCs. This phenomenon is observed at the early stage of implantation (i.e., within 4 days of implantation).

A previously developed mathematical model describing the in vivo kinetics of macrophage fusion to form FBGCs on biomaterials was employed to provide insights into the effect of peptide identity on the kinetics of FBGC formation. FBGC cell size distributions on all samples were measured at 4, 7, 14, and 21 days post-implantation. The FBGC cell size-distribution equation was fitted to the measured results of each sample at each retrieval time to obtain values for p and 1/a. Values for p increased with increasing implantation time for all samples except for that of the “no grafted peptide” controls. Thus, these results indicate that the probability of cell fusion increased with increasing implantation time. The calculations also showed that the density of adherent macrophages that participate in the FBGC formation was significantly higher for mPEGmA-co-Ac-co-TMPTA gels grafted with G₃RGDG, G₃PHSRNG, and G₃PHSRNG₆RGDG than that for gels grafted with G₃RDGG nonspecific controls and gels without peptide grafting.

This Example shows that the hydrogels of the present invention can be used to support peptide, proteins, and the like, within a modified, three-dimensional hydrogel matrix.

TABLE 7
Total and different leukocyte concentration in the inflammatory exudate
of mPEGmA-co-AC-co-TMPTA networks grafted with various
fibronectin-derived oligopeptides.^{a}

| | Implantation | Cell concentration (×10 cells/μl) | | | |
|---|---|---|---|---|---|
| Peptide | (days) | Total | Lymphocyte | Monocyte | PMN |
| G₃RGDG | 4 | 127 ± 25 | 71 ± 22 | 56 ± 5^{b} | 0 ± 0 |
| | 7 | 67 ± 13 | 24 ± 4^{c} | 43 ± 9^{b} | 0 ± 0 |
| | 14 | 74 ± 18 | 21 ± 4 | 53 ± 25 | 0 ± 0 |
| | 21 | 31 ± 8^{c,d,b} | 27 ± 8^{c,b} | 5 ± 1^{d} | 0 ± 0 |
| G₃PHSRNG | 4 | 63 ± 32 | 25 ± 22^{b} | 38 ± 17^{b} | 0 ± 0 |
| | 7 | 61 ± 9 | 25 ± 6 | 36 ± 3^{b} | 0 ± 0 |
| | 14 | 56 ± 19 | 33 ± 15 | 24 ± 4 | 0 ± 0 |
| | 21 | 77 ± 2^{c} | 69 ± 2^{c,d} | 7 ± 3 | 0 ± 0 |
| G₃RGDG₆PHSRNG | 4 | 129 ± 52 | 29 ± 10^{b} | 99 ± 62^{b} | 1 ± 1 |
| | 7 | 68 ± 23 | 24 ± 6 | 44 ± 17^{b} | 0 ± 0 |
| | 14 | 57 ± 12 | 21 ± 9 | 36 ± 10 | 0 ± 0 |
| | 21 | 74 ± 2^{c} | 67 ± 3^{c,d} | 7 ± 3 | 0 ± 0 |
| G₃PHSRNG₆RGDG | 4 | 109 ± 16 | 53 ± 14^{b} | 56 ± 5^{b} | 0 ± 0 |
| | 7 | 49 ± 11^{d} | 21 ± 8 | 28 ± 3^{b,d} | 0 ± 0 |
| | 14 | 87 ± 23 | 38 ± 12 | 49 ± 29 | 0 ± 0 |
| | 21 | 60 ± 11 | 55 ± 8 | 5 ± 3^{d} | 0 ± 0 |
| G₃RDGG | 4 | 91 ± 11 | 51 ± 1^{b} | 40 ± 10^{b} | 0 ± 0 |
| | 7 | 66 ± 16 | 30 ± 11 | 36 ± 6^{b} | 0 ± 0 |
| | 14 | 48 ± 9^{d} | 23 ± 6^{d} | 25 ± 6 | 0 ± 0 |
| | 21 | 35 ± 10^{c,d,b} | 32 ± 9^{c,b} | 4 ± 2^{d} | 0 ± 0 |
| No grafted | 4 | 94 ± 32 | 42 ± 27^{b} | 52 ± 16^{b} | 0 ± 0 |
| peptide | 7 | 41 ± 10 | 11 ± 2^{b} | 30 ± 6^{b} | 0 ± 0 |
| | 14 | 89 ± 21 | 56 ± 18 | 33 ± 15 | 0 ± 0 |
| | 21 | 63 ± 4 | 55 ± 4 | 7 ± 2^{d} | 0 ± 0 |
| Empty cage | 4 | 135 ± 22 | 129 ± 22 | 6 ± 1 | 0 ± 0 |
| | 7 | 42 ± 8^{d} | 38 ± 8^{d} | 4 ± 1 | 0 ± 0 |
| | 14 | 51 ± 10^{d} | 35 ± 6^{d} | 15 ± 10 | 0 ± 0 |
| | 21 | 82 ± 22^{d} | 80 ± 25^{d} | 2 ± 2 | 0 ± 0 |

^{a}All values expressed in (mean ± s.e.m., (n = 3).
^{b}Represents p < 0.05 vs. respective values of “empty cage” controls.
^{c}Represents p < 0.05 vs. respective values of “no grafted peptide” controls.
^{d}Represents p < 0.05 vs. respective values at day 4 of the same sample type.

TABLE 8
Adherent macrophage density on cage-implanted mPEGmA-co-AC-co-
TMPTA networks grafted with various fibronectin-derived oligopeptides^{a}

| | Adherent macrophage density (×10 macrophages/mm²) at | | | | | |
|---|---|---|---|---|---|---|
| | various post-implantation time (days) | | | | | |
| Peptide | 4 | 7 | 14 | 21 | 35 | 70 |
| G₃RGDG | 138 ± 22^{b} | 85 ± 12^{b,c} | 33 ± 12^{b,c} | 15 ± 3^{c} | 14 ± 2^{c} | 4 ± 2^{c} |
| G₃PHSRNG | 124 ± 12^{b} | 57 ± 10^{b,c} | 31 ± 11^{b,c} | 10 ± 0^{c} | 9 ± 1^{c} | 4 ± 2^{c} |
| G₃EGDG₆PHSRNG | 126 ± 8^{b} | 58 ± 12^{b,c} | 23 ± 4^{b,c} | 14 ± 4^{c} | 6 ± 5^{c} | 0 ± 0^{c} |
| G₃PHSRNG₆RGDG | 183 ± 27^{b} | 69 ± 6^{b,c} | 30 ± 5^{b,c} | 16 ± 4^{c} | 12 ± 5^{c} | 3 ± 1^{c} |
| G₃RDGG | 75 ± 16 | 36 ± 5^{c} | 15 ± 3^{c} | 15 ± 6^{c} | 9 ± 3^{c} | 3 ± 2^{c} |
| No grafted peptide | 74 ± 26 | 37 ± 4^{c} | 14 ± 2^{c} | 19 ± 3^{c} | 6 ± 3^{c} | 1 ± 1^{c} |

^{a}All values expressed in mean ± s.e.m. (n = 3).
^{b}Represents p < 0.05 vs. respective values of “no grafted peptide” controls.
^{c}Represents p < 0.05 vs. respective values at day 4 of the same sample type.

### Example 4

### Interpenetrating Membranes Comprising Modified Hydrogels

Interpenetrating networks (IPNs) are hydrogels synthesized by reacting a first polymer around a second material to form an intermeshing structure. IPNs are free of cross-linkers used to create other biomedical hydrogels. In addition to the benefit of being free of potentially toxic chemicals used in conventional cross-linking procedures, photopolymerization has the advantages that the desired amount of drug can be easily loaded into the matrix, and the cross-linking density, which can affect the drug release rate, can be controlled. Furthermore, IPNs can be formed in situ and used in places less suitable for prefabricated materials.

The focus of this Example was to investigate the swelling and drug release kinetics of gelatin-based IPNs of varying gelating backbone modification, weight percent of gelatin, pH, and the molecular weight of polyethylene glycol diacrylate (PEGdA). Based on our results, these IPNs are quite suitable for tissue scaffolds and drug release vehicles.

Polyethyleneglycol (PEG) (Aldrich; 2, 4.6, and 8 kDa) was modified with acrylolyl chloride (Aldrich) and TEA (Aldrich) in a 1:4:6 molar ratio at room temperature for 3 hours to produce polyethylene glycol diacrylate (PEGdA). The final PEGdA product purity was checked with the same reverse phase HPLC system as used in Example 1. The elution time of the PEGdA was approximately 13.2 minutes with a purity of approximately 100 wt % PEGdA.

Monomethoxypolyethyleneglycol (mPEG) (Fluka; 2 kDa) was modified with acetic anhydride (Aldrich) and DMSO (Fisher) in a 1:80:140 molar ratio at room temperature to form an mPEG monoaldehyde (mPmA). The reaction takes 8 to 24 hours and was monitored periodically with HPLC. The mPmA had an elution time of approximately 11.9 minutes and a purity close to 75 wt % mPmA. The compositions of PEGdA and mPEGmonoaldehyde were also confirmed with ¹H-NMR.

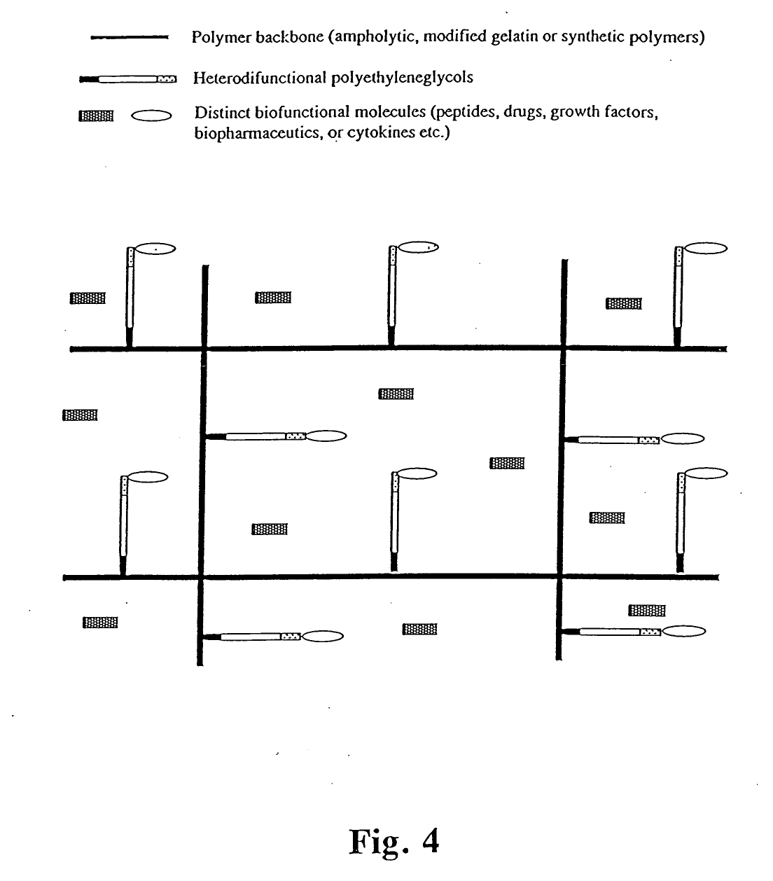

Gelatin (G) (Sigma, Type A: from porcine skin, 300 bloom) lysyl groups were modified with EDTAD in a 1:0.034 weight ratio for 3 hours at pH=10 to form EDTAD-G (EG). Gelatin lysyl groups were also modified with mPmA and sodium cyanoborohydride (NaCNBH₃) (Aldrich) in a 1:0.66:0.186 weight ratio for 24 hours at 50 to 60° C. to form mPmiAG (FIG. 4). EG was further modified with mPmA in a procedure similar to the mPmiAG procedure. The percent of the gelatin lysyl residues modified by EDTAD and/or mPmA was determined using the trinitrobenzene sulfonic acid spectrophotometric method. The IPNs used in this study were prepared from the same modified gelatins.

IPNs were created using modified and unmodified gelatin, PEGdA (2, 4.6, or 8 kDa molecular weight), initiator (2,2-dimethoxy-2-phenylacetophenone, DMPA), and a long wavelength UV source. Gelatin was dissolved in deionized water with heat (80° C.) to form a 20 wt % gelatin solution. PEGdA was dissolved in deionized water, without heat, in an aluminum foil wrapped glass vial to form a 100 wt % PEGdA solution. The gelatin solution was then added to the PEGdA solution and the mixture was agitated thoroughly. DMPA was then added to the gelatin/PEGdA mixture and this final mixture was again agitated and then heated (80° C.) throughout the rest of the procedure. IPNs were created through injection molding. The final gelatin/PEGdA/DMPA mixture was injected with a Pasteur pipette into a Teflon mold that was clamped between 2 glass slides. The mold has the approximate dimensions of 20 mm long by 10 mm wide by 1.6 mm thick. The mold/IPN mixture was then irradiated with UV light from the top and bottom for approximately 3 minutes. During this time, the UV light initiates the cross-linking of PEGdA, entrapping the gelatin within the PEGdA cross-links. The mold/IPN was allowed to cool before the IPN was removed from the mold.

IPNs were named based on the weight percent of gelatin, the type of gelatin, the weight percent of PEGdA, and the molecular weight of the PEGdA used to synthesize the IPN. For example, 4G6P2k indicates 40 wt % gelatin, 60 wt % PEGdA, 2 kDa PEGdA. The following key describes the code used to identify IPN formulations.

Key: Each formulation is identified by a code of the formula “XYZk”, where X is the wt % gelatin, Y is the type of gelatin, Z is the wt % PEGdA, and k is the molecular weight of the PEGdA:

$$\begin{matrix}X = wt \% gelatin \\ 4 = 40 wt \% \\ 6 = 60 wt \%\end{matrix}$$

$$\begin{matrix}Y = type of gelatin \\ G = gelatin \\ EG = EDTAD \text{-} modified gelatin \\ mPMaG = mPmA \text{-} modified gelatin \\ mPmAEG = mPmA / EDTAD \text{-} modified gelatin\end{matrix}$$

$$\begin{matrix}Z = wt \% PEGdA \\ 4 = 40 wt \% \\ 6 = 60 wt \%\end{matrix}$$

$$\begin{matrix}k = molecular weight PEGdA \\ 2 k = 2000 Da \\ 4.6 k = 4 \text{,} 600 Da \\ 8 k = 8 \text{,} 000 Da\end{matrix}$$

The swelling/degradation kinetics of the IPNs were characterized by weighing swollen IPNs at predetermined times (up to 8 weeks). The IPNs were added to test tubes containing 5 ml deionized water with environmental pHs of 4.5, 7.0, and 7.4. The test tubes were then placed in water baths at 37° C. At the predetermined times, the samples were removed with extreme caution from the test tubes using a bent spatula, blotted dry, weighed, and then placed back in the same test tube. This was done until the sample had degraded completely or until the sample had degraded into too many pieces and they could no longer be removed from the test tube. The swelling weight ratio at each time point for each IPN was calculated as: (Wₛ−Wₒ/Wₒ), where Wₛ is the weight of the swollen IPN and Wₒ is the original weight of the IPN. The maximum swelling weight ratio that occurred over 8 weeks and the time it occurred was calculated (Rₘₐₓ, Tₘₐₓ). The last attainable swelling weight ratio (due to IPN degradation) and the time it occurred was also calculated (R_{fail}, T_{fail}).

The level of host biocompatability and inflammatory reaction of the IPNs was determined via the in vivo subcutaneous cage implant system described in the previous Examples. IPNs were placed inside cylindrical (1 cm diameter by 3.5 cm long) medical grade stainless steel wire mesh cages. These cages along with empty cages, controls, were implanted subcutaneously at the back of 3-month old female Sprague-Dawley rats. Inflammatory exudates that collected in the cages were withdrawn at 4, 7, 14, and 21 days post-implantation and analyzed for the quantitative evaluation of cellular and humoral response to the IPN samples using standard hematology techniques. Using these techniques the distributions of polymorphonuclear leukocyte (PMN), lymphocyte, and monocyte subpopulations in the exudates were determined. In addition to the host response, the degradation of the IPNs was determined as percent weight lost ((final IPN weight/initial IPN weight)×100).

The IPNs fabricated as described hereinabove were opaque, flexible, rubbery, and slightly tacky. The opacity increased with decreasing gelatin concentration and with increasing PEGdA molecular weight. Increasing the gelatin concentration increased the flexibility and the tackiness of the IPN. The flexibility of the IPNs also seemed to increase with increasing PEGdA molecular weight.

The mechanical properties of the IPNs were tested using ASTM testing standards. The IPNs for mechanical testing were made in a similar fashion as stated above, however the molds used were made of polydimethylsiloxane and the IPN final dimensions were 280 mm thick, 11 mm gauge length, and 2 mm neck width (the dimensions required for ASTM D38-98 type IV specimens). The IPNs were subjected to tensile testing per ASTM D638-98 standards, using an Instron Model 5548 testing machine.

The preliminary mechanical tests indicated that the average Young's Modulus of the 4G6P2K IPNs was 1.26±0.14 N/nm². The ultimate tensile stress and strain were 0.39±0.10 N/nm² and 0.49±0.07 mm/mm, respectively.

Swelling/degradation studies (Table 9) showed that increasing the molecular weight of the PEGdA to 4.6 kDa and 8 kDa increased the maximum swelling ratio (Rₘₐₓ). Modifying gelatin with EDTAD and mPmA did not appear to affect Rₘₐₓ. The time to Rₘₐₓ (Tₘₐₓ) increased with increasing PEGdA molecular weight and by modifying gelatin. The swelling ratio at failure (R_{fail}) decreased when the wt % of gelatin was decreased from 60 to 40 when PEGdA molecular weight was held constant at 2 kDa. In addition, when the PEGdA molecular weight was 2 kDa and the gelatin was 60 wt %, modifying the gelatin did not improve R_{fail}. The time to reach R_{fail} (T_{fail}) was not affected by increasing the molecular weight of PEGdA or by modifying the gelatin. Table 9 shows Rₘₐₓ, Tₘₐₓ, R_{fail}, and T_{fail} for each composition of IPNs tested at pH=7. These trends were comparable at pH of 4.5 and 7.4 (results not shown). The release kinetics and bioactivity of human serum albumin, chlorhexidine gluconate, and b-FGF (1%) from these IPNs in vitro are currently being quantified.

TABLE 9
Rₘₐₓ, Tₘₐₓ, R_{fail}, and T_{fail} for Various IPN Formulations at pH 7.0

| Formulation | Rₘₐₓ | Tₘₐₓ | R_{fail} | T_{fail} |
|---|---|---|---|---|
| 6G4P2K | 0 | 0 | −0.736 | 9 |
| 4G6P2K | 0.754 | 1 | <0.444 | >1344 |
| 6G4P4.6K | 1.733 | 225.667 | <0.505 | >1344 |
| 4G6P4.6K | 2.2 | 4.333 | <1.538 | >1344 |
| 6G4P8K | 1.646 | 225 | 0.758 | 451.333 |
| 4G6P8K | 3.911 | 227.333 | <1.542 | >1344 |
| 6EG4P2K | 0.128 | 1 | −0.214 | 336.33 |
| 4EG6P2K | 0.712 | 27 | <0.532 | >1344 |
| 6EG4P4.6K | 2.288 | 35.333 | <0.818 | >1344 |
| 4EG6P4.6K | 1.452 | 17.667 | <0.773 | >1344 |
| 6EG4P8K | 2.639 | 5 | 0.794 | 960 |
| 4EG6P8K | 3.026 | 1 | −0.252 | 1097.3 |
| 6mPmAG4P2K | 0.469 | 1.667 | <−0.23 | >1344 |
| 4mPmAG6P2K | 0.891 | 672 | <0.827 | >1344 |
| 6mPmAG4P4.6K | 2.467 | 226.667 | <0.621 | >1344 |
| 4mPmAG6P4.6K | 2.578 | 616 | <2.445 | >1344 |
| 6mPmAG4P8K | 3.854 | 336.667 | 2.717 | 944 |
| 4mPmAG6P8K | 6.075 | 337 | <2.626 | >1344 |
| 6mPmAEG4P8K | 2.715 | 2.333 | <1.265 | >1344 |
| 4mPmAEG6P8K | 4.224 | 192.333 | <1.472 | >1344 |

Forty (40) wt % gelatin, 60 wt % PEGdA 2 kDa (4G6P2K) IPNs were used in a preliminary in vivo study. The presence of a high concentration of PMN in the exudates, relative to the control, indicates an acute inflammation response, due to the onset of implantation, which attenuates with time. Acute inflammation is followed by a high concentration of monocytes and lymphocytes in the exudates, chronic inflammation. The study showed that there was a statistically higher inflammatory response to the IPNs after 4, 7, and 14 days of implantation compared to the empty cage controls. The study also revealed that almost 70% of the sample mass was lost after 4 days, and decreased another 10% after 21 days.

Currently an in vivo study is underway. The study is investigating the drug release and effect of dexamethasone from IPNs of composition 40 wt % gelatin, 60 wt % PEGdA 2 kDa, and 60 wt % PEGdA 2 kDa.

The Example illustrates that IPNs made according to the present invention can serve as tissue scaffolds and drug delivery vehicles.

### Example 5

### Synthesis of Ac-PEG-COOH in the Construction of Cell Non-Adhesive Substrates

To synthesize COOH-PEG-Ac with high purity on a large scale (i.e., grams), HO-PEG-EtAt (2 KDa), 1 eq. mol PEG (2 KDa) was dissolved in dry THF and 1.7 eq. mol sodium hydride (NaH) and stirred at room temperature for 1 h in an argon bag (Inflatable Glove Chamber, Instrument for Research and Industry X-37-37). 1.7 eq. mol ethyl bromoacetate was added to the solution, stirred under argon at room temperature for 2 h, and filtered. The filtrate was precipitated in cold hexane, filtered and dried in a vacuum oven to obtain a mixture of PEG and HO-PEG-EtAt.

The dried mixture of PEG and HO-PEG-EtAt was then dissolved in deionized water and 1.7 eq. mol 1N NaOH² was added. The solution was stirred for 2 h, adjusted to pH 12.5, and extracted by methylene chloride once. The aqueous phase solution was adjusted to pH 3 with 1N HCl, and extracted again with methylene chloride. The organic phase solution was evaporated under vacuum using a Rotavapor (BUCCI R-114, Switzerland), dried in a vacuum oven overnight. This yielded HO-PEG-COOH (2K Da) with a purity of 99% in 15% yield.

To synthesize COOH-PEG-Ac, 1 eq. mol HO-PEG-COOH (2058 Da) was dissolved in dry THF with 2.3 eq. mol acryloyl chloride and 2.5 eq. mol triethylamine. The solution was stirred at room temperature for 4 h and filtered. The filtrate was precipitated in cold hexane, filtered and dried in a vacuum oven to obtain COOH-PEG-Ac¹⁴ with a purity of around 98% and a final yield of around 10% of the starting material, PEG 2K. The overall reaction is shown below.

The purified COOH-PEG-Ac 2K was then polymerized into trimethylolpropane triacrylate (TMPTA) based networks. (See Table 10.) MPEG-Ac 2K or 454 (added to minimize non-specific peptide/protein absorption) and COOH-PEG-Ac 2K were conjugated into networks based on a previously developed polymer network that has been described in detail.¹⁰⁻¹³ Varying concentrations of each of MPEG-Ac 2K or 454 and COOH-PEG-Ac-2K were tested (see Table 11).

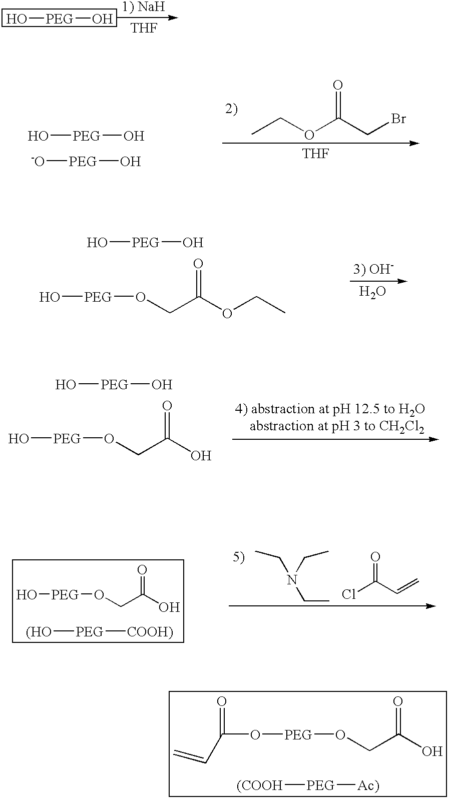

Briefly, MPEG-Ac 2K was dissolved in TMPTA at 80° C. followed by the addition of COOH-PEG-Ac 2K. The mixture was vortexed thoroughly, followed by the addition of a photo initiator, 2,2-dimethoxy-2-phenyl-actone (3.6 mg/ml). The heated solution was poured into circular polytetrafuoroethylene molds measuring 0.6 cm in diameter and 0.1 cm in thickness, and clamped between two pieces of cover glass covered by two glass slides. The assembly was heated at 80° C. for 2 min followed by UV (BLACK-RAY, B 100 AP, UVP, maximum intensity at 365nm, 21,700 mWatts/cm² at 2.5cm) treatment for 40 sec at 80° C. The network discs were removed from the molds and post-cured by UV light for another 5 min, put in DMF for 5 h to leach out unreacted materials, and transferred to sterilized water for storage.

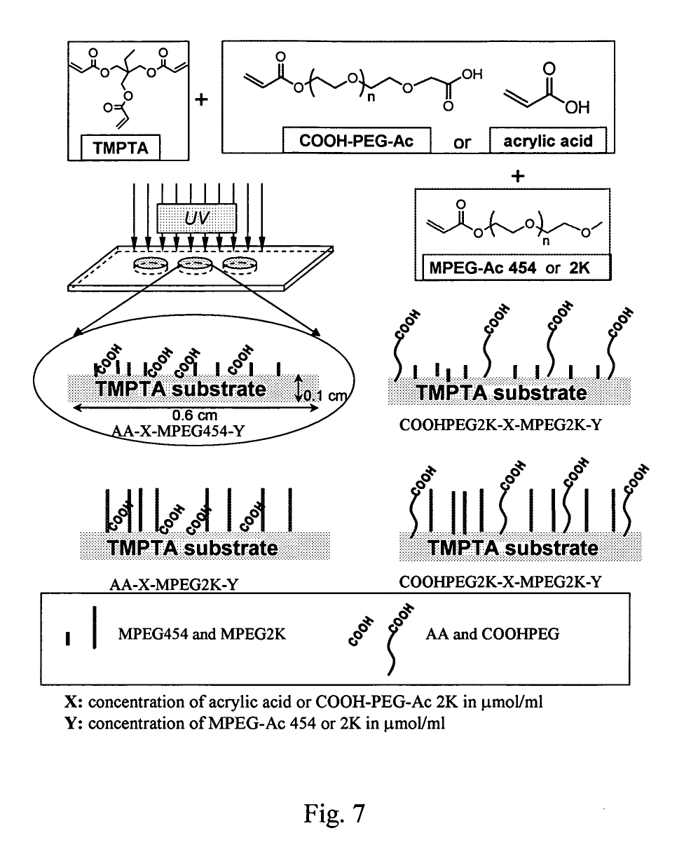

See FIG. 7 for a schematic illustration of how the TMPTA networks having COOH groups as grafting sites for peptides are fabricated.

TABLE 10
Compositions of the TMPTA networks containing COOH-PEG-Ac 2K
and MPEG-Ac 454 or 2K.

| | | Molar | | |
|---|---|---|---|---|
| | MW | Concentration | Wt/Vol. | |
| Substance | (g/mol) | (μmol/ml) | Conc. (/ml) | |
| COOH-PEG-Ac 2K | 2112 | 0.2 | 0.0004 | g |
| | | 2 | 0.004 | g |
| | | 20 | 0.04 | g |
| | | 200 | 0.4 | g |
| MPEG-Ac 454 | 454 | 100 | 41.55 | μl |
| | | 200 | 83.1 | μl |
| | | 400 | 166.2 | μl |
| MPEG-Ac 2K | 2000 | 100 | 0.2 | g |
| | | 200 | 0.4 | g |
| | | 400 | 0.8 | g |

TABLE 11
TMPTA Networks Compositions.

| | Molecular | Molar | | |
|---|---|---|---|---|
| | Weight | Conc. | Weight/Volume | |
| Substance | (g/mol) | (μmol/ml) | Conc. (/ml) | |
| Acrylic Acid | 72 | 20 | 1.36 | μl |
| MPEG-Ac 454 | 454 | 100 | 41.55 | μl |
| | | 200 | 83.1 | μl |
| | | 400 | 166.2 | μl |
| MPEG-Ac 2K | 2000 | 100 | 0.2 | g |
| | | 200 | 0.4 | g |
| | | 400 | 0.8 | g |

Tables 12 and 13 summarize the types of networks constructed. Four concentrations were used for networks containing COOH-PEG-Ac 2K (i.e. 0.2, 2, 20 and 200 μmol/ml) and one concentration of MPEG-Ac 2K (20 μmol/ml). The concentration of COOH-PEG-Ac 2K was the determining parameter for the network formulations, as networks with COOH-PEG-Ac 2K at concentrations greater than 200 μmol/ml were opaque and cracked easily. Networks containing COOH-PEG-Ac 2K at a concentration of 20 μmol/ml was the highest concentration that maintained the integrity of the networks.

TABLE 12
Types of TMPTA-Based Networks Containing COOH-PEG-Ac 2K and
MPEG-Ac 454 or 2K

| | | |
|---|---|---|
| COOHPEG2K-0.2-MPEG454-100 | COOHPEG2K-0.2-MPEG454-200 | COOHPEG2K-0.2- |
| | | MPEG454-400 |
| COOHPEG2K-2-MPEG454-100 | COOHPEG2K-2-MPEG454-200 | COOHPEG2K-2- |
| | | MPEG454-400 |
| COOHPEG2K-20-MPEG454-100 | COOHPEG2K-20-MPEG454-200 | COOHPEG2K-20- |
| | | MPEG454-400 |
| COOHPEG2K-200-MPEG454-100 | COOHPEG2K-200-MPEG454-200 | COOHPEG2K-200- |
| | | MPEG454-400 |
| COOHPEG2K-0.2-MPEG2K-100 | COOHPEG2K-0.2-MPEG2K-200 | COOHPEG2K-0.2- |
| | | MPEG2K-400 |
| COOHPEG2K-2-MPEG2K-100 | COOHPEG2K-2-MPEG2K-200 | COOHPEG2K-2- |
| | | MPEG2K-400 |
| COOHPEG2K-20-MPEG2K-100 | COOHPEG2K-20-MPEG2K-200 | COOHPEG2K-20- |
| | | MPEG2K-400 |
| COOHPEG2K-200-MPEG2K-100 | COOHPEG2K-200-MPEG2K-200 | COOHPEG2K-200- |
| | | MPEG2K-400 |

TABLE 13
Types of TMPTA-Based Networks Containing Acrylic Acid and MPEG-
Ac 454 or 2K

| | | |
|---|---|---|
| AA-20-MPEG454-100 | AA-20-MPEG454-200 | AA-20-MPEG454-400 |
| AA-20-MPEG2K-100 | AA-20-MPEG2K-200 | AA-20-MPEG2K-400 |

To determine the non-adhesiveness of the networks containing COOH-PEG-Ac 2K, a total of 50,000 human dermal fibroblasts in 1 ml fibroblast basal medium (FBM, Cambrex) supplemented with basic human fibroblast growth factor, insulin, and 5% fetal bovine serum (Cambrex) were cultured with the networks in a 48-well culture plate with a seeding density of 50,000 cells/cm². Tissue culture polystyrene (TCPS) was used as positive control. After 2, 24 or 48 h, networks were fixed with Wrights' stain (Sigma) and the adherent fibroblasts were imaged using a computer-assisted video analysis system (MetaMorph V.4. 1) coupled to an inverted light microscope (PHOTOMETRICS, SenSys). The adherent fibroblast cell density and morphology were evaluated. All experimental results were expressed in mean±standard deviation (SD). Each sample was independently repeated 3 times (n=3). Comparative analyses were performed with Statview® 4.5 using analysis of variance and Fisher's protected least significant difference test at 95% confidence level (p<0.05). See Tables 14 and 15, below.

PEG has two identical terminal HO groups, both of which can undergo reactions with other reagents. Molar ratios of other reagents, versus PEG play an important role in determining the conversion ratio of PEG bis-carboxylate (PEG-bis-COOH) and HO-PEG-COOH from PEG. In the reaction scheme illustrated in the immediately preceding chemical reaction, there were five steps in the pathway. Steps 1, 2 and 3 were essentially one step because step 1 was to ionize PEG in order to facilitate step 2. Step 3 was to hydrolyze the ethyl acetate group to obtain COOH group. In the synthesis of COOH-PEG-Ac, the intermediate product, HO-PEG-COOH, was a mixture with PEG and PEG-bis-COOH. The goal then was to maximize the conversion to PEG-bis-COOH from PEG (60%), while maintaining product purity.

In order to obtain HO-PEG-COOH with high purity, the starting material (i.e. PEG) and the side product (i.e. PEG-bis-COOH) must be minimized. The abstraction technique, described herein, succeeded in separating HO-PEG-COOH and PEG-bis-COOH from PEG. At pH 12.5, the PEG terminal COOH groups were ionized and remained in aqueous phase, whereas PEG was abstracted to methylene chloride due to the high solubility of PEG in methylene chloride. However, no pH point could be found to completely separate HO-PEG-COOH from PEG-bis-COOH. Therefore, the formation of PEG-bis-COOH should be minimized. A series of molar ratios were experimented before a preferred molar ratio of 1:1.7 for PEG versus other reagents was selected for steps 1-3. Applying this preferred molar ratio, only PEG and HO-PEG-COOH resulted without the formation of PEG-bis-COOH after step 3. The conversion ratio of HO-PEG-COOH was around 15%. After the abstraction procedure, HO-PEG-COOH resulted with a purity of around 99% (See FIG. 7) and a final yield of around 15%. Starting with 20 g PEG, around 2 g HO-PEG-COOH with high purity was obtained, a large amount compared to PEG derivatives purified by any chromatogram technique that works on 1 to 100 mg scale. The purity of HO-PEG-COOH also ensured the purity of COOH-PEG-Ac and the series of hPEG derivatives described in Example 6.

The goal for the fibroblast adhesion Example was to determine optimum formulations of the substrates that do not crack and are transparent, have minimum concentration of MPEG-acrylate 454 or 2K for minimum fibroblast adhesion, and also have a maximum concentration of acrylic acid or acrylate-PEG-COOH 2K for peptide grafting.

TMPTA based networks containing COOH-PEG-Ac 2K at 200 mol/ml were of poor physical property (i.e. opaque and cracked), hence were excluded from the fibroblast cell adhesion study. The molecular weight of MPEG-Ac, the concentration of COOH-PEG-Ac 2K and the concentration of MPEG-Ac all played significant roles in mediating fibroblast adhesion (See all of Tables 13, 14, and 15). Specifically, the adherent cell density decreased with increasing concentrations of COOH-PEG-Ac 2K, MPEG-Ac 454 and/or 2K. The adherent cell density also decreased with increasing molecular weight of MPEG-Ac. Cell densities on all TMPTA networks were significantly lower than those on TCPS after 24 h of culture. Cell densities on all TMPTA networks decreased as time increased. At the same concentration of MPEG-Ac 454 or 2K, fibroblast cell density was higher on TMPTA networks containing acrylic acid than that on TMPTA networks containing COOH-PEG-Ac at the same concentration.

Thus networks containing COOH-PEG-Ac 2K at the highest concentration (i.e., 20 μmol/ml) and MPEG-Ac 454 or 2K at a concentration of 200 μmol/ml support minimum non-specific protein adsorption while providing a sufficient number of surface COOH groups as grafting sites for peptides.

TABLE 14
Adherent fibroblast cell density on TMPTA containing networks containing
COOH-PEG-Ac 2K and MPEG-Ac 454 or 2K (cells/mm²):

| | | | | | | | | |
|---|---|---|---|---|---|---|---|---|
| 0.2:100 | 0.2:200 | 0.2:400 | 2:100 | 2:200 | 2:400 | 20:100 | 20:200 | 20:400 |

COOH-PEG2K μM:MPEG-Ac-454 μM

| | | | | | | | | | |
|---|---|---|---|---|---|---|---|---|---|
| 2 h | 56 ± 9 | 66 ± 11 | 61 ± 7 | 38 ± 8 | 32 ± 8 | 36 ± 10 | 23 ± 10 | 22 ± 8 | 19 ± 9 |
| 24 h | 23 ± 14 | 22 ± 9 | 12 ± 7 | 32 ± 11 | 04 ± 6 | 06 ± 4 | 32 ± 11 | 04 ± 6 | 06 ± 4 |
| 48 h | 36 ± 8 | 29 ± 16 | 15 ± 21 | 10 ± 7 | 17 ± 5 | 17 ± 12 | 21 ± 4 | 20 ± 18 | 02 ± 6 |

COOH-PEG2K μM:MPEG-Ac-2K μM

| | | | | | | | | | |
|---|---|---|---|---|---|---|---|---|---|
| 2 h | 20 ± 3 | 12 ± 3 | 11 ± 2 | 35 ± 4 | 38 ± 8 | 10 ± 5 | 11 ± 3 | 13 ± 11 | 06 ± 7 |
| 24 h | 21 ± 17 | 14 ± 9 | 19 ± 5 | 25 ± 10 | 08 ± 8 | 07 ± 6 | 14 ± 4 | 16 ± 19 | 06 ± 3 |
| 48 h | 32 ± 13 | 18 ± 8 | 21 ± 13 | 12 ± 14 | 18 ± 10 | 00 ± 1 | 02 ± 2 | 12 ± 11 | 03 ± 2 |

Substrate Controls

| | TMPT | TCPS |
|---|---|---|
| 2 h | 63 ± 13 | 135 ± 0 |
| 24 h | 61 ± 22 | 142 ± 0 |
| 48 h | 71 ± 17 | 209 ± 0 |

TABLE 15
Adherent fibroblast cell density on TMPTA networks containing AA and
MPEG-Ac 454 or 2K (cells/mm²):

| | AA μM:MPEG-Ac 454 μM | | | Substrate Controls | |
|---|---|---|---|---|---|
| | 20:100 | 20:200 | 20:400 | TMPTA | TCPS |
| 2 h | 64 ± 25 | 69 ± 14 | 55 ± 16 | 73 ± 14 | 149 ± 25 |
| 24 h | 59 ± 40 | 77 ± 26 | 62 ± 46 | 53 ± 22 | 164 ± 12 |
| 48 h | 33 ± 13 | 54 ± 9 | 45 ± 45 | 39 ± 16 | 176 ± 14 |

AA μM:MPEG-Ac 2K μM

| | 20:100 | 20:200 | 20:400 |
|---|---|---|---|
| 2 h | 77 ± 37 | 29 ± 17 | 5 ± 1 |
| 24 h | 53 ± 22 | 41 ± 12 | 0 ± 0 |
| 48 h | 13 ± 7 | 15 ± 17 | 0 ± 0 |

*All values are expressed in cells/mm² (mean ± SD, n = 3)

All experimental results were expressed in mean±standard deviation (SD). Each sample was independently repeated 3 times (n=3). Comparative analyses were performed with Statview® 4.5 using analysis of variance and Fisher's protected least significant difference test at 95% confidence level (p<0.05).

### Example 6

### Solution Phase PEG Peptide Conjugation

As described in the previous Example, networks containing α-carboxy-ω-acryloyl-PEG (COOH-PEG-Ac) 2K or acrylic acid at a concentration of 20 μmol/ml and α-methoxy-ω-acryloyl-PEG (MPEG-Ac) 454 or 2K at a concentration of 200 μmol/ml were selected to perform peptide grafting and cell adhesion studies. The formulations of trimethylolpropane triacrylate (TMPTA)-based networks were optimized with minimum non-specific protein adsorption and maximum COOH groups as grafting sites for peptides. To ensure successful grafting of peptides on the networks, solution phase polyethylene glycol (PEG) activation and peptide conjugation was explored.

The synthesis of PEG peptide conjugates is essential in that it provides a means for introducing bioactive molecules in the construction of drug delivery systems or bioactive materials using polymer materials. Among various polymers, PEG has been commonly used as a carrier system for biological proteins, due to PEG's low toxicity, low immunogenicity, and good solubility in both aqueous and organic solvents. PEGylation of proteins have been well documented. However, relatively few studies have focused on the PEGylation of small peptides, possibly due to the assumption that modification of a small bioactive peptide with a large molecule, such as PEG, would result in a loss of activity of the peptide.

All starting compounds were used as received without additional purification. All chemicals were purchased from Aldrich except for those specified. All peptides were synthesized by the University of Wisconsin Biotechnology Center Peptide Synthesis Facility (Madison, Wis.). THF was dried prior to use. Both ACS grade DMF and anhydrous DMF were used. If not noted, ACS grade DMF was used. Intermediate and final products were characterized by a reverse phase high performance liquid chromatogram (HPLC) system (Gilson, 10% to 100% acetonitrile at a flow rate of 1 ml/min in 30 min coupled with a UV/Vis detector and an evaporative light scattering detector (ELSD). In this HPLC system, compounds with earlier elution times are of higher hydrophilicity. All PEG derivatives were synthesized from PEG 2K.

Synthesis of α-Hydrox-ω-N-Succinimidyl-Acetate-PEG (HO-PEG-NSu):

Two methods were used to synthesize HO-PEG-NSu. In the first method, a total of 0.206 g HO-PEG-COOH (2058 Da, 0.1 mmol) was dissolved in 2 ml THF or DMF followed by the addition of 0.0288 g N-hydroxysuccinimide (N-HOSu, 115 Da, 0.25 mmol) and 0.0515 g 1,3-dicyclohexylcarbodiimide (DCC, 206 Da, 0.25mmol), and stirred under argon for 2 h. The solution in THF was dried using a drying procedure in which the solution was filtered and the filtrate was precipitated in cold hexane, filtered again and dried in a vacuum oven to obtain HO-PEG-NSu. The solution in DMF was not processed, because precipitating PEG derivatives from DMF results in low yield. Also the purpose of the procedure was not to obtain a solid form of HO-PEG-NSu, but to investigate whether the terminal COOH group of PEG could be activated in DMF.

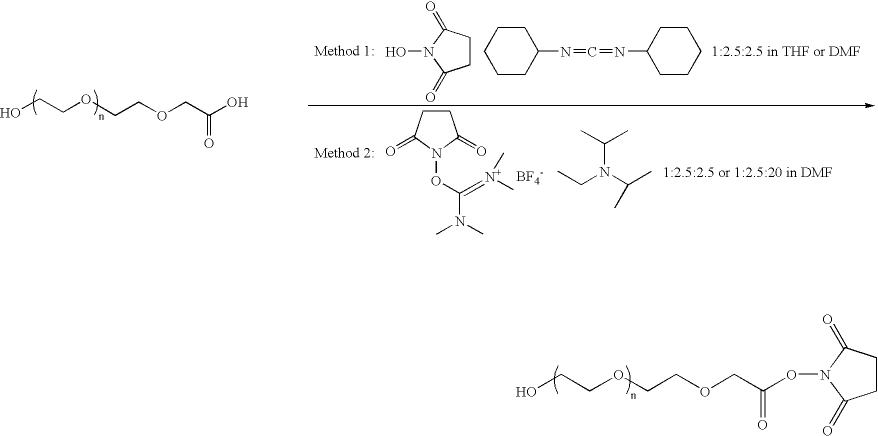

### Synthesis of HO-PEG-NSu 2K

In the second method, 20 mg HO-PEG-COOH (0.01 mmol) was dissolved in 0.4 ml DMF followed by the addition of 7.5 mg N,N,N′,N′-Tetramethyl-O—(N-succinimidyl)uronium (TSTU, 301 Da, 0.025 mmol) and 4.3 ul or 434 ul N,N-diisopropyl ethylamine (DIPEA, 129 Da, 0.742 g/ml, 0.025 or 2.5 mmol). The solutions were stirred at room temperature and monitored with HPLC up to 21h. These solutions were not processed for the same reasons as described above.

Conjugation of HO-PEG-NSu with lysine (K):

Conjugation with K was first performed to determine optimum conditions for grafting K onto COOH-presenting substrate surfaces. K possesses a side chain NH₂ group. Fluorospheres with surface aldehyde groups can be conjugated with the side chain NH₂ group of K grafted on network surfaces. The fluorescence labeling method was used to validate the peptide grafting procedure in solid phase. Four batches of 21.6 mg HO-PEG-NSu 2K (2155 Da, 10 μmol) were dissolved in 0.432 ml DMF, followed by the addition of K (146.2 Da, 3.8 mg, 30 μmol) directly or dissolved in 76 μl 0.1 N 2-(N-morpholino)ethanesulfonic acid (MES) buffer before the addition. A total of 5.2 μl DIPEA (30 μmol) was then added to two of the solutions. See Table 16. MES and DIPEA were used in a combination of four conditions to explore whether and how these two conditions would contribute to the conjugation of HO-PEG-NSu with K.

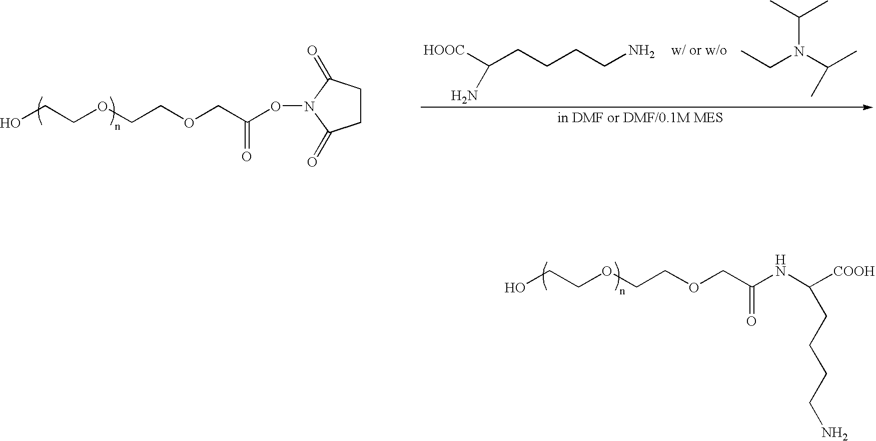

### Conjugation of HO-PEG-NSu with K

TABLE 16
Specific amounts of reagents in the conjugation of HO-PEG-NSu with K

| Reaction | HO-PEG-COO-NSu | DMF | K | 0.1M MES | DIPEA |
|---|---|---|---|---|---|
| 1 | 21.6 mg (10 μmol) | 0.432 ml | 38 mg (30 μmol) | — | — |
| 2 | 21.6 mg (10 μmol) | 0.432 ml | 38 mg (30 μmol) | — | 5.2 μl (30 μmol) |
| 3 | 21.6 mg (10 μmol) | 0.432 ml | 38 mg (30 μmol) | 76 μl | — |
| 4 | 21.6 mg (10 μmol) | 0.432 ml | 38 mg (30 μmol) | 76 μl | 5.2 μl (30 μmol) |

The solutions were stirred at room temperature and monitored with HPLC up to 5 h.

Conjugation of HO-PEG-NSu with RGDW in Four Different Solvents (DMF, DMF/PBS, THF/PBS):

The chemistry of conjugating PEG with peptide sequences differs from that with single amino acids because peptides are more complex. Hence conjugation of HO-PEG-NSu with peptides was also explored. To conjugate HO-PEG-NSu with Arg-Gly-Asp-Trp (RGDW), four conditions were explored using a combination of two types of solvents (THF and DMF) and PBS buffer. Two types of solvents were used to examine the reactivity of the activated COOH group in the two solvents. PBS buffer was used to examine whether buffer could facilitate the dissolution of peptides in the conjugation with HO-PEG-NSu. Four batches of 21.6 mg HO-PEG-NSu (10 μmol) were dissolved in 0.432 ml THF or DMF, followed by the addition of 10.7 mg RGDW (532.6 Da, 20 μmol) directly or dissolved in 107 μl pH 7.4 PBS buffer before the addition to the solution. A total of 3.5 μl DIPEA (20 μmol) was then added in each one of the solutions (Table 17). The solutions were stirred under argon and monitored by HPLC up to 1 h.

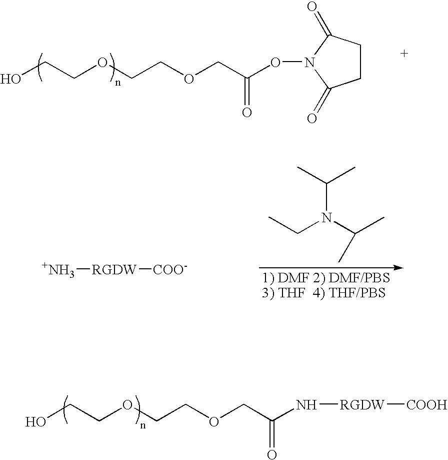

### Conjugation of HO-PEG-NSu with RGDW

TABLE 17
Specific amounts of reagents in the conjugation of HO-PEG-NSu with RGDW

| Reaction | HO-PEG-COO-NSu | THF | DMF | RGDW | PBS | DIPEA |
|---|---|---|---|---|---|---|
| 1 | 21.6 mg (10 μmol) | 0.432 ml | — | 11.7 mg (20 μmol) | — | — |
| 2 | 21.6 mg (10 μmol) | 0.432 ml | — | 11.7 mg (20 μmol) | 107 μl | 3.5 μl (20 μmol) |
| 3 | 21.6 mg (10 μmol) | — | 0.432 ml | 11.7 mg (20 μmol) | — | — |
| 4 | 21.6 mg (10 μmol) | — | 0.432 ml | 11.7 mg (20 μmol) | 107 μl | 3.5 μl (20 μmol) |

Test of the Stability of the Acrylate Group in DMF or DMF/PBS in the Presence of DIPEA:

To construct networks with covalently conjugated peptides, two methods were explored. The first one method was to construct networks with COOH-PEG-Ac resulting in substrate with surface COOH groups. The substrate surface COOH was then activated and conjugated with peptides. The second method was to find a way to synthesize Ac-PEG-peptide and construct Ac-PEG-peptide directly into the network without further treatment in solid phase. To synthesize Ac-PEG-peptide conjugates, the stability of the arcylate group in the presence of DIPEA in different solutions was critical. To test the stability of the acrylate group, two batches of 0.1 g MPEG-Ac (2 KDa, 0.05 mol) were dissolved in 1 ml DMF or a co-solvent of 1 ml DMF and 0.5 ml PBS followed by the addition of 13 μl DIPEA (0.075 mol). The solutions were stirred under argon and monitored by HPLC for up to 18 h.

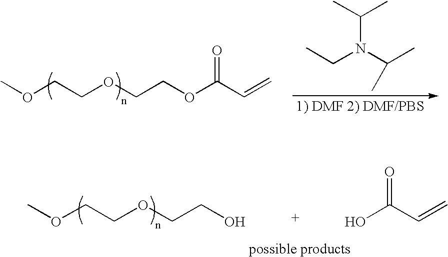

### Stability of acrylate Group in DMF or DMF/PBS Co-Solvent

Conjugation of HO-PEG-NSu with RGD and Test of the Stability of HO-PEG-NSu in the Presence of DIPEA in DMF:

In the series of reactions conjugating HO-PEG-NSu with RGDW, hydrolysis dominated in the presence of PBS. Whether the product was pure α-hydrox-ω-RGDW-acetate-PEG (HO-PEG-RGDW) or a combination of HO-PEG-RGDW and HO-PEG-COOH must be further examined. Hence, two reactions were performed, one using RGD and the other without RGD, to examine the stability of NSu group in the presence of DIPEA.

In trial runs, two batches of 21.6 mg HO-PEG-NSu (10 μmol) were dissolved in DMF (0.432 ml). One solution was added with 3.46 mg RGD (346.2 Da, 10 μmol) followed by the addition of 1.74 μl DIPEA (10 μmol). Only 1.74 μl DIPEA was added to the other solution. The two solutions were stirred at room temperature and monitored by HPLC for up to 1 h.

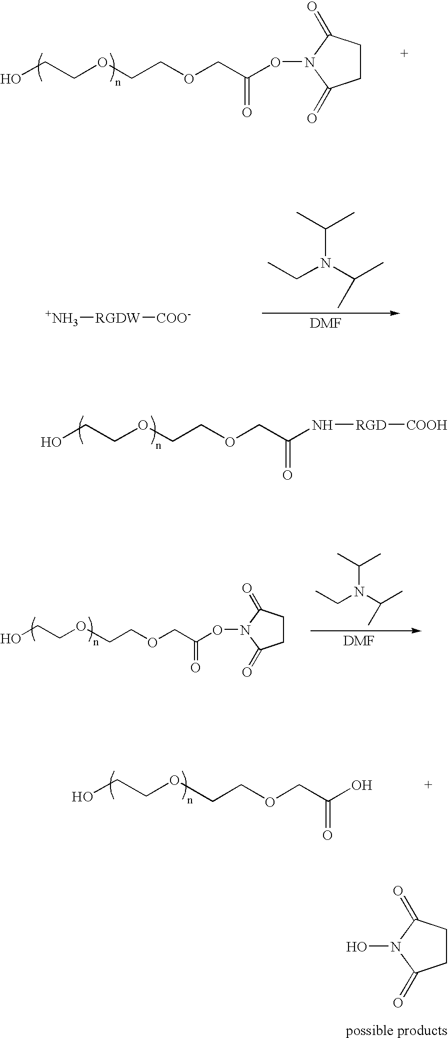

### Conjugation of HO-PEG-NSu with RGD and Test of the Stability of NSu

Conjugation of HO-PEG-NSu with Trp and Test of the Stability of HO-PEG-NSu in the Presence of DI]PEA in DMF at a Very Anhydrous Conditions:

The NSu moiety hydrolyzed in the presence of DI[PEA in reactions described above. It was hypothesized that trace amounts of water in the system hydrolyzed HO-PEG-NSu in the presence of DIPEA. Therefore, two reactions were designed to be performed under anhydrous conditions in which one reaction was with Trp and the other was without Trp in the presence of DIPEA. All the experimental materials for the reaction including round-bottom (RB) flasks, syringes and needles were dried at 80° C. overnight. The flasks were capped with septum lids and cooled down while being purged with argon. Two batches of 0.216 g HO-PEG-NSu (0.1I mmol) were transferred to dried and cooled RB flasks, capped with a septum and purged with argon for 10 min. Anhydrous DMF was obtained from a solvent system (Glasscontour) and transferred to the two RB flasks via a syringe. One of the flasks was added with 27.9 mg Trp (0. 15 mmol). Both flasks were purged with argon followed by the addition of 26 μl DIPEA (0.15 mmol) via a syringe. The two solutions were stirred, purged with argon and monitored up to 17 h. The precautions taken in this experiment will be referred as “anhydrous conditions” and will be used thereafter to avoid redundancy. 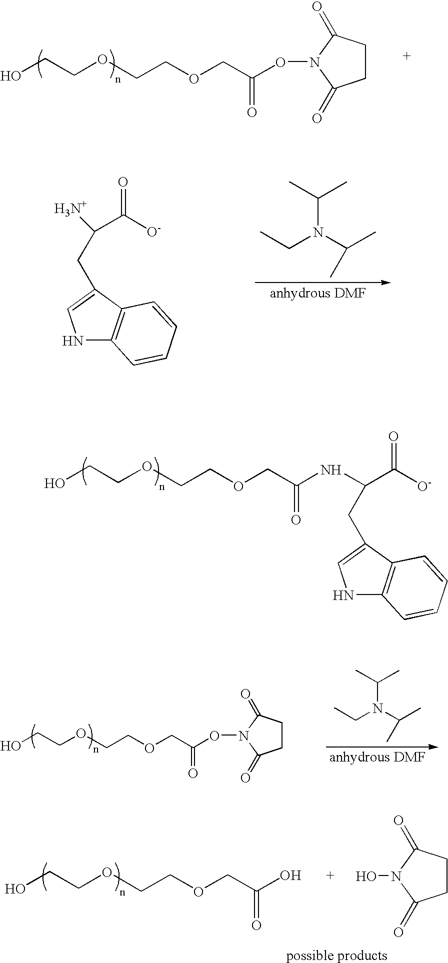
Conjugation of HO-PEG-NSu with a Series of Peptides:

Anhydrous DMF was used to conjugate HO-PEG-NSu with a series of peptides to examine whether the same conjugation conditions as described above applied to all peptide identities.

The molar ratios of all the conjugations were 1:1.5:1.5 for HO-PEG-NSu:peptide:DIPEA. Under anhydrous conditions, a total of 387.9 mg HO-PEG-NSu (0.18 mmol) was dissolved in 7.76 ml anhydrous DMF from the solvent system, added to 9 vials with an aliquot of 8.7 ml for each vial, followed by the addition of 9 types of peptides, 0.03 mmol for each peptide (i.e., RGD: 10.4 mg, PHSRN: 609 Da, 18.3 mg, PHSRNRGD: 938 Da, 28.1 mg, PHSRNG₃RGD: 1109 Da, 33.3 mg, PHSRNG6RGD: 1281 Da, 38.4 mg, PHSRNGP₄GRGD: 1441 Da, 43.2 mg, G₇: 418 Da, 12.5 mg, G₃RGDG: 648 Da, 17.2 mg or G₃PHSRNG: 835 Da, 25.1 mg) to each vial. The solutions were stirred and purged with argon for 19 h, precipitated in cold ether and dried in a vacuum oven for overnight. The reactions were monitored by HPLC and the final products were characterized with MS-spec (Ion Spec, Fourier Transform Mass Spectrometer): 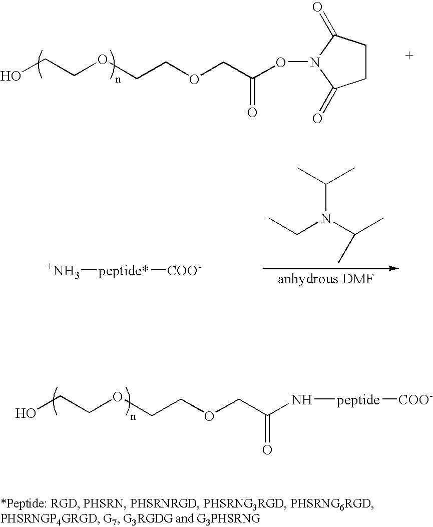
Synthesis of α-Acryloyl-ω-N-Succinimidyl-Acetate-PEG (Ac-PEG-NSu):

Another route to create substrates with surface-bound peptide was to synthesize Ac-PEG-peptide first and directly construct into networks without further treatment in the solid phase. To synthesize Ac-PEG-peptide, synthesis of Ac-PEG-NSu is the first step. Two methods were explored to synthesize Ac-PEG-NSu. In the first method, 1.67 g HO-PEG-NSu (0.775 mmol) was dissolved in 9 ml THF followed by the addition of 0.189 ml acryloyl chloride (AC, 90.5 Da, 1.114 g/ml, 2.325 mmol) and 0.377 ml triethylamine (TEA, 101.19 Da, 0.728 g/ml, 2.71 mmol), stirred under argon at room temperature for 2 h, dried using the drying procedure to obtain Ac-PEG-NSu.

In the second method, HO-PEG-NSu was acrylated before being precipitated from THF. A total of 6.17 g HO-PEG-COOH (3 mmol) was dissolved in 30 ml dry THF followed by the addition of 0.863 g N-HOSu (7.5 mmol) and 1.545 g DCC (7.5 mmol). The solution was stirred under argon at room temperature for 2 h, followed by the addition of 0.73 ml AC (9 mmol) and 1.46 ml TEA (10.5 mmol). The reaction was stirred under argon at room temperature for 2 h, dried using the drying procedure to obtain Ac-PEG-NSu:

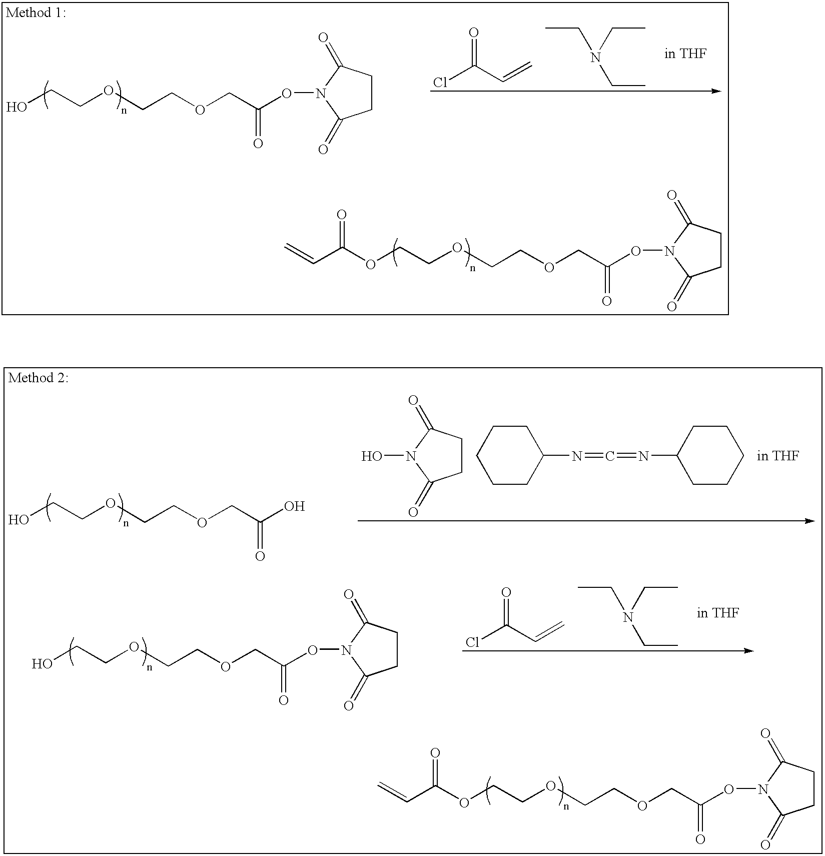

### Synthesis of HO-PEG-NSu

Conjugation of Ac-PEG-NSu with RGD or RGDW:

To synthesize α-acryloyl-ω-RGDW-acetate-PEG (Ac-PEG-RGDW), 88.4 mg Ac-PEG-NSu (2210 Da, 0.04 mmol) was dissolved in DMF followed by the addition of 32 mg RGDW (0.06mmol) and purged with argon. A total of 10.4 ul DIPEA (0.06 mmol) was added to the solution via a syringe. The solution was stirred and purged with argon for 1 h, precipitated in cold ether, filtered and dried in a vacuum oven overnight to obtain Ac-PEG-RGDW:

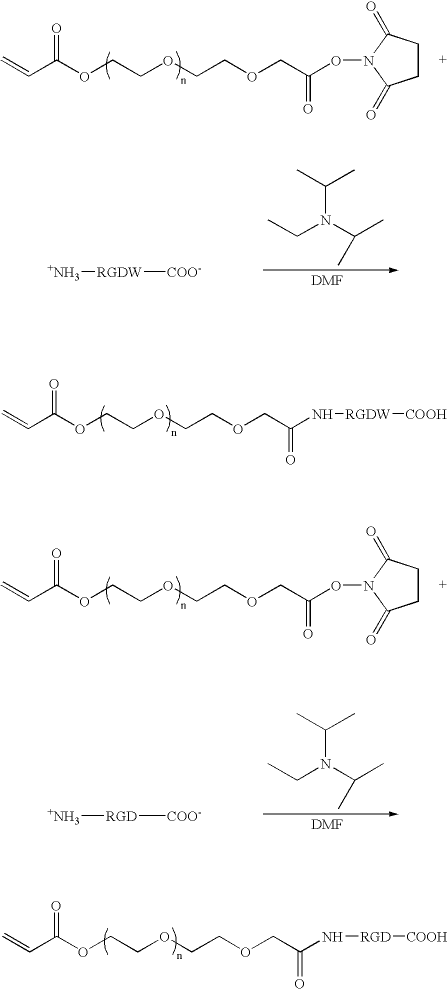

### Conjugation of Ac-PEG-NSu with RGDW or RGD

Synthesis of Ac-PEG-RGD was the same as described above with exception that different amounts of reagents were used (i.e. 222 mg Ac-PEG-NSu (0.1 mmol), 52 mg RGD (0.15 mmol) and 26 ul DIPEA (0.15 mmol) ) (FIG. 4-9).

Conjugation of Ac-PEG-NSu with RGD, Trp, RGDW and PHSRN in Anhydrous DMF and Under Sonication:

During the reaction with RGDW and RGD described above, aggregates with a diameter of 0.1˜0.5 mm formed in both solutions. The presence of a hydrophobic group (i.e., Ac) and a hydrophilic group (i.e., RGD or RGDW) could have facilitated the self-assembly of the molecules. To resolve the problem, sonication was introduced into the reaction system. Under anhydrous conditions, four batches of 0.221 g Ac-PEG-NSu (0.1 mmol) were dissolved in 4.42 ml anhydrous DMF, followed by the addition of 30.6 mg Trp (0.15mmol), 69 mg RGD (0.2 mmol), 80 mg RGDW (0.15 mmol) or 91.4 mg PHSRN (0.15 mmol) respectively. Containers of the four solutions were placed in a sonicator and sonication was applied 5 min every 20 min. The solutions were stirred while being purged with argon at room temperature, and monitored by HPLC up to 17 h, precipitated with cold ether, filtered and dried in a vacuum oven overnight:

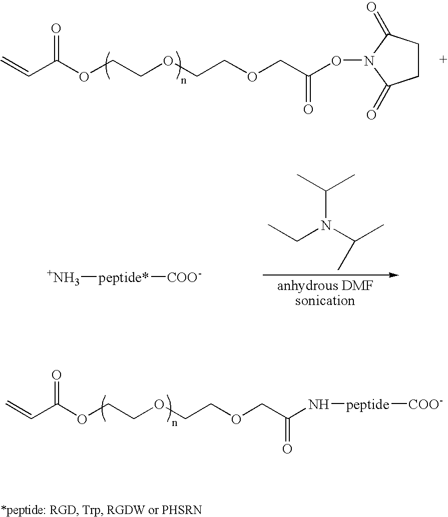

HO-PEG-COOH was conjugated with peptides in solution phase under the same conditions applied to the peptide grafting procedure described herein. As described above, a synthesis scheme was developed and optimized to obtain HO-PEG-COOH with high purity (99%) in a large scale (i.e., 2 g of product from 20 g PEG). Both N-HOSu and TSTU were employed to activate COOH groups. N-HOSu was used to determine its efficiency in activating COOH using THF as a solvent. Whereas activating COOH with TSTU cannot proceed in THF, THF is the preferred solvent in PEG derivatization because PEG or PEG derivatives can be precipitated easily from THF with cold hexane, whereas precipitating PEG or PEG derivatives from DMF results in low yield. TSTU was used to probe the validity of activating procedure in the established protocol noted earlier. The activated HO-PEG-COOH should be the same product, HO-PEG-NSu. HO-PEG-NSu was then either acrylated or conjugated with K. Acrylating HO-PEG-NSu allowed us to explore whether the substrate activating procedure could be moved to the solution phase. Conjugating with K was to investigate the validity of the grafting procedure. The reaction scheme is as follows: 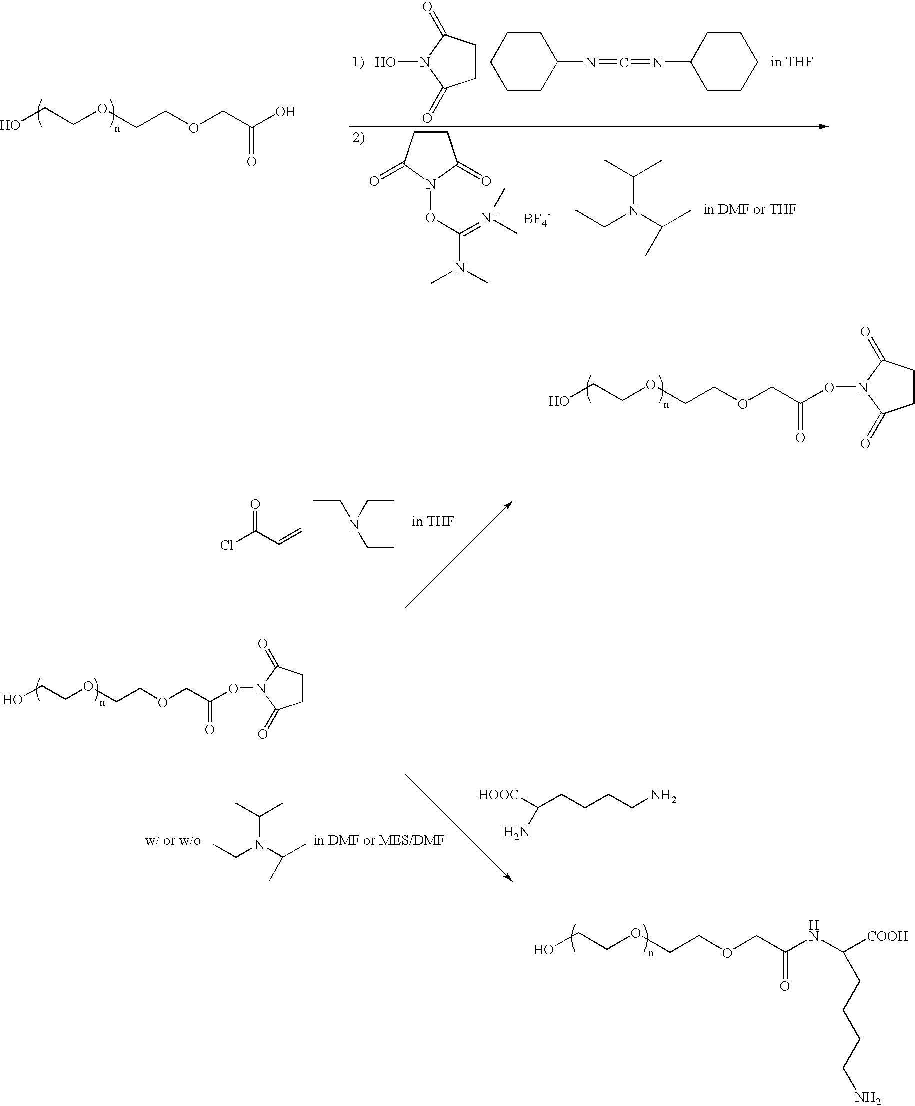
Scheme for Activating HO-PEG-COOH in Solution Phase to Obtain HO-PEG-NSu, and then Acrylating HO-PEG-NSu or Conjugating HO-PEG-NSu with K

Examining the structure of HO-PEG-COOH and HO-PEG-NSu, HO-PEG-NSu was seen to be more hydrophobic than HO-PEG-COOH. The elution time for HO-PEG-COOH (2K Da) in a 30-min HPLC run was around 8.1 min. A compound eluted at 12.0 min represented the formation of HO-PEG-NSu. The formation of HO-PEG-NSu was also confirmed by ¹H and 13C NMR (See Table 18). From the HPLC chromatograms of the reactions (data not shown), HO-PEG-COOH was converted to HO-PEG-NSu with N-HOSu and DCC resulting in a conversion ratio of around 98% after 1 h in THF and after 30 min in DMF.

TABLE 18
NMR characterization for PEG derivatives and the intermediate products in the
construction of cell non-adhesive networks
Chemical shifts of designated carbon (in superscript) in
compounds with the following general structure:

| | X-C(α₁)H₂C(β₁)H₂O(CH₂CH₂O)nC(β2)H₂C(α₂)H₂—Y | | | | | | | | | | Terminal functional group | |
|---|---|---|---|---|---|---|---|---|---|---|---|---|
| | nCH2 | C(a1) | C(b1) | C(a2) | C(b2) | C(1) | C(2) | C(3) | C(4) | C(5) | X | Y |
| MPEG-Ac | 71 | 64 | 68 | 67 | 71 | 165 | 128 | 131 | 58 | — | —OC(1)OC(2)HC(3)H₂ | —O—C(4)H₃ |
| COOH-PEG- | 71 | 65 | 69 | 71 | 71 | 169 | 128 | 131 | 73 | 173 | —OC(1)OC(2)HC(3)H₂ | —OC(4)H₂—C(5)OOH |
| Ac | | | | | | | | | | | | |
| HO-PEG- | 71 | 62 | 69 | 71 | 71 | 73 | 173 | — | — | — | —OH | —OC(1)H₂—C(2)OOH |
| COOH | | | | | | | | | | | | |
| HO-PEG- COO-NSu | 71 | 62 | 69 | 71 | 71 | 71 | 178 | 170 | 26 | — | —OH | 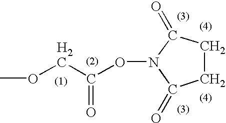 |

As for the reaction with TSTU and DIPEA, the reaction did not proceed in THF. In DMF the reaction was slow prior to 2 h. However, the conversion of HO-PGE-COOH to HO-PEG-NSu increased dramatically to 90% at 4 h when the molar ratio of HO-PEG-COOH:TSTU:DIPEA was 1:2:2 and then decreased to 73% after 21 h. When excess amounts of DIPEA was used (i.e. 1:2:200 for HO-PEG-COOH:TSTU:DIPEA), the conversion reached a maximum of 72% after 4h and decreased to 57% after 21 h. Excess amounts of DIPEA was used in the established protocol for grafting peptides to surface COOH groups to be described in chapter 5. The same condition used in solution phase resulted in lower conversion-of HO-PGE-COOH to HO-PEG-NSu over long period of time indicating that the conditions used for activating substrate surface COOH groups in the established protocol was not efficient.

A common obstacle in PEGylateing proteins or peptides is the transformation of the hydroxy terminal of PEG to an active group. Two important classes of methods are used. One involves alkylating PEGs and the other involves acylating PEGs. Alkylating PEG includes PEG-aldehyde that gives a permanent linkage after Shiff base formation followed by cyanoborohydride reduction. This method is slow and pH sensitive. PEG-tresyl chloride activation is another method of synthesizing alkylated PEGS. However, the chemistry or conjugation and the conjugation products are not unique and well defined. Epoxy PEG has been used, but the reactivity is low and the specificity is not certain since hydroxy groups may also react. A main method to acylate PEGs is to conjugate N-HOSu with carboxylated PEGs, a method employed in our synthesis scheme. It is important to know that the distance between the active ester (—NSu) and PEG ether can vary in different available products; by up to four methylene units. This has profound influence on the reaction towards amino groups on the protein or peptide as well as water. As an example, the t^{1/2} (i.e. half life) of the hydrolysis of PEG-O—CH₂—CH₂—CH₂—COOSu is 23 h, while that for PEG-O—CH₂—COOSu is 0.75 h.

N-HOSu activated PEG is used extensively in modifying enzymes (e.g., lipase, superoxide dismutase, methioninase, adn arginine deiminase) to increase enzyme stability, decrease thermal sensitivity and enhance process versatility and was also modified by PEG-NSu. PEG-NSu was also used to modify doxorubicin and monoclonal antibodies for extend antitumor effects. HO-PEG-NSu was very sensitive to water and hydrolyzed readily. Storage at 4° C. under argon prevented the hydrolysis.

Conjugation of HO-PEG-NSu with K:

In conjugating NSu activated PEG carboxylate with amine groups on peptide or protein, a combination of DMF and 0.1M MES solvents were used. DMF was employed to dissolve PEG and MES buffer was used to dissolve peptides. DIPEA, an organic amine, was used to deionize the amine group in an amino acid making the amine group a better nucleophile to substitute NSu and conjugate to the PEG terminal COOH group. The incorporation of DIPEA in the conjugation of HO-PEG-NSu with peptides facilitates the reaction. A combination of these conditions was used to explore the function of MES and DIPEA in the conjugation of activated PEG COOH group with amine groups on K.

The conjugation did not proceed without MES and DIPEA. For the reaction with DIPEA alone, after 30 min a peak eluting at around 8.3 min dominated in a 30-min HPLC. For the reaction with MES alone, a peak eluting at around 9.7 min dominated. For reaction with both MES and DIPEA, there were three peaks eluting at 8.1 min, 9.2 min and 9.7 min respectively.

There were two NH2 groups on K, both of which could conjugate with activated COOH. The pKa of the side chain NH₂ was 10.6 whereas the pKa of the terminal NH₂ was around 9. For the reaction with DIPEA alone, DIPEA deprotonized NH₂ making it a better nucleophile to attack NSu activated COOH. Because the pKa of the terminal NH₂ is lower, the terminal NH₂ should be deprotonized the first. Hence, the majority of activated COOH should conjugate with terminal NH₂. For the reaction with MES alone, the NH₂ on the side chain of K is more flexible, thus interacted more readily with the NSu activated COOH group. In the reaction where both DIPEA and MES were used, both the terminal and the side NH₂ groups could conjugate with the COOH group. To verify the above speculations, two reactions in which either MES or DIPEA was used were repeated using K with the side chain NH₂ protected (i.e. N-ε-(tert-butoxycarbonyl)-L-Lysine (N-ε-tBoc-K)). In the reaction with DIPEA, after 5 min of reaction, a single peak eluted off at around 8.6 min. The same peak eluted off after 30 min in the reaction with MES. The side chain NH₂ group was protected in these two reactions and could not conjugate with the COOH, leaving terminal NH₂ the only group to be able to conjugate with HO-PEG-NSu. HO-PEG-K-N-ε-tBoc eluted off at 8.6 min whereas the product resulted from conjugation of HO-PEG-NSu with K in the presence of DIPEA eluted at 8.3 min, and the product resulted from conjugation of HO-PEG-NSu with K in the presence of MES eluted at 9.7 min. The protected K was more hydrophobic rendering the conjugated product, HO-PEG-K-N-ε-tBoc, more hydrophobic than HO-PEG-K. Therefore, the product in the reaction with DIPEA must be K conjugated to HO-PEG-NSu through the terminal NH₂ and the product in the reaction with MES must be K conjugated to HO-PEG-NSu through the side chain NH₂. The result of the two series of reactions verified that in the presence of DIPEA, the reaction was much faster, only 5 min in case of K. The conjugation site was more specific to the terminal NH₂ because the pKa of the terminal NH₂ is lower than that of the side chain NH₂ in K. The finding indicated that when HO-PEG-NSu conjugates with the series of peptides containing side amine groups (i.e., arginine in RGD and PHSRN), the binding site would be more localized to the terminal NH₂.

Conjugation of HO-PEG-NSu with RGDW in Four Different Solvents (DMF, DMF/PBS, THF, THF/PBS):

To construct networks with covalently tethered peptides, two methods were explored. One method was to construct networks with Ac-PEG-COOH resulting in substrates with surface COOH groups. Surface COOH groups on the substrate were then activated and conjugated with peptides using conditions optimized in the solution phase. Another method was to find a way to synthesize Ac-PEG-peptide and construct Ac-PEG-peptide into the network directly without further treatment in solid phase.

The series of reactions performed were to examine the reactivity of the activated COOH group with RGDW. The result would provide reference for the condition of substrate peptide grafting and the solution phase synthesis of Ac-PEG-RGDW. Another buffer, PBS, was used in two of the reactions to examine whether a biological buffer would facilitate dissolution of the peptide for conjugation with HO-PEG-NSu. All reactions completed within 30 min as indicated by the disappearance of the peak at around 11.6 min (HO-PEG- NSu) and the appearance of a peak at around 8.1-8.6 min (HO-PEG-COOH or HO-PEG-RGDW). HO-PEG-RGDW formed in solvents of DMF, DMF/PBS or THF, because the peak was associated with a characteristic absorbance of Trp at 280 nm. However, in THF/PBS, HO-PEG-COOH formed since no UV absorbance was associated with the peak. In the presence of PBS, hydrolysis of the NSu groups dominated. The reaction proceeded in THF indicating that this solvent may be used in the synthesis of PEG peptide conjugates.

Test of the Stability of Acrylate Group in DMF, or DMF/PBS, or THF in the Presence of DIPEA:

One method to create substrates with surface tethered peptides was to find a way to synthesize Ac-PEG-peptide and construct Ac-PEG-peptide into the network directly without further treatment in solid phase. To make Ac-PEG-peptide, the stability of the acrylate group in the presence of DIPEA was critical in the conjugation reaction. The acrylate group was stable in all the solvents up to 18 h as the peak at around 11.6 min (HO-PEG- NSu) remained unchanged in the HPLC chromatograms (data not shown).

Conjugation of HO-PEG-NSu with RGD and Test of the Stability of HO-PEG-NSu in the Presence of DIPEA in DMF:

As described earlier, the elution times for HO-PEG-COOH and HO-PEG-RGDW were very close. Because in the presence of PBS, hydrolysis of HO-PEG-NSu occurred, two reactions were performed to examine whether the peak around 8.1-8.6 min was pure HO-PEG-RGDW or a combination of HO-PEG-RGDW and HO-PEG-COOH. In the reaction using RGD, HO-PEG-RGD formed after 30 min demonstrated by the disappearance of the peak at around 11.6 min and the appearance of a peak at around 8.2 min with weak UV absorbance at 200 nm (data not shown). In the reaction using no peptide, HO-PEG- NSu was not stable in DMF in the presence of DIPEA demonstrated by the disappearance of the peak at around 11.6 min and the appearance of a peak at around 8.1 min without any UV absorbance at 200 nm.

It was proposed that the presence of H₂O was the reason NSu group hydrolyzed in the presence of DIPEA by the following reaction scheme: 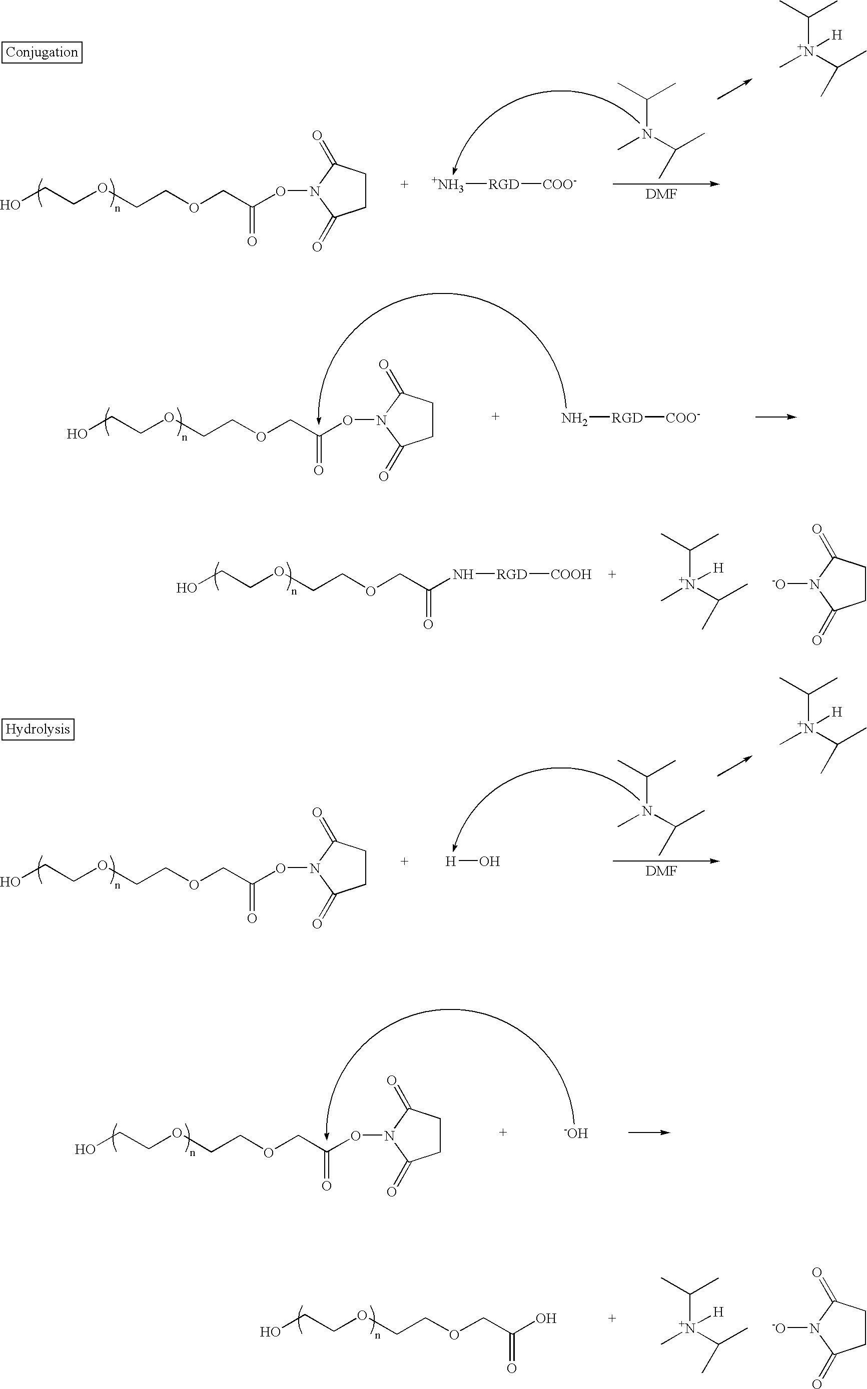
Conjugation of HO-PEG-NSu with Trp and Test of the Stability of HO-PEG-NSu in the Presence of DIPEA in DMF at Very Anhydrous Conditions:

To verify the proposed mechanism presented above, two reactions were performed in anhydrous DMF involving the addition of Trp or the absence of Trp. The conjugation of HO-PEG-NSu with Trp completed in 30 min in the presence of DIPEA demonstrated by the disappearance of the peak at around 11.6 min and the appearance of a peak at around 8.1 min associated with a strong UV absorbance at 280 nm. The compound remained stable up to 17 h. HO-PEG-Nsu was stable in andydrous DMF in the presence of DIPEA up to 17 h. The result from these two reactions showed that the reaction system for conjugating HO-PEG-NSu with peptides in the presence of DIPEA must be anhydrous.

Conjugation of HO-PEG-NSu with Peptides:

Anhydrous DMF was used to conjugate HO-PEG-NSu with peptides to examine whether the conjugation would proceed for all peptide identities at the same condition described previously.

All reactions took place in 3 h demonstrated by the disappearance of the peak at around 11.6 min and the appearance of new peaks at around 8.1 min for RGD, G₃RGDG and G₇, and at around 10.8 min for peptides containing PHSRN (i.e., PHSRN, PHSRNRGD, PHSRNG₃RGD, PHSRNG6RGD, PHSRNGP₄GRGD and G₃PHSRNG) (See Table 19). The dried products were small in quantity, and were difficult to measure. In order to prepare samples for MS-spec characterization, the dried products were dissolved in water directly and the concentrations of the products were semi-quantified by HPLC. Based on the ELSD channel signals which correlates the mass concentration of the molecules on HPLC chromatograms, concentrations of the products were estimated and were diluted to 0.1˜1 mg/ml for MS-spec. The MS-spec chromatograms verified the formation of PEG-peptide conjugates.

TABLE 19
Summery of the elution time on HPLC chromatograms, peak molecular
weights (Mp) as determined from the MS-spec and the estimated
molecular weights of the PEG-peptide conjugates

| | Elution | | |
|---|---|---|---|
| PEG-peptide conjugates | time (min) | Mp | MW estimated |
| HO-PEG-RGD | 8.21 | 2431 | 2386 |
| HO-PEG-PHSRN | 10.58 | 2721 | 2649 |
| HO-PEG-PHSRNRGD | 10.72 | 2775 | 2978 |
| HO-PEG-PHSRNG₃RGD | 10.65 | 3078 | 3149 |
| HO-PEG-PHSRNG₆RGD | 10.59 | 3249 | 3321 |
| HO-PEG-PHSRNGP₄GRGD | 10.63 | 3409 | 3481 |
| HO-PEG-G₃RGDG | 7.93 | 2628 | 2615 |
| HO-PEG-G₃PHSRNG | 10.50 | 2878 | 2878 |
| HO-PEG-G7 | 8.10 | 2256 | 2458 |

Thus, a complete schematic reaction route of synthesizing PEG-peptide conjugates from HO-PEG-COOH is briefly summarized as follows:
HO-PEG-COOH Activation: 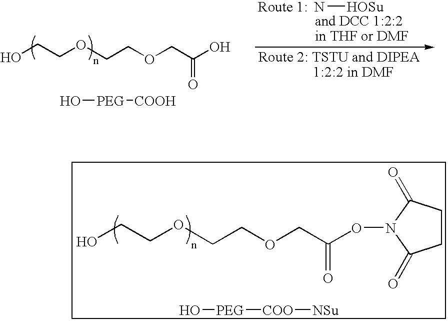
PEG-RGD Conjugation: 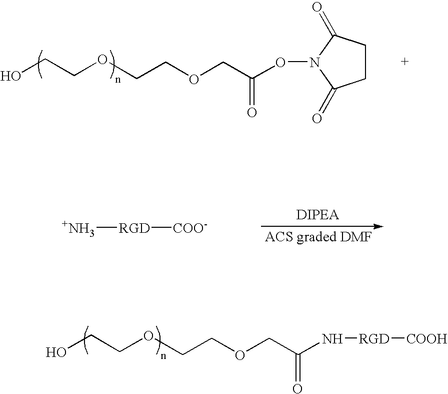
Problem (Hydrolysis of NSu): 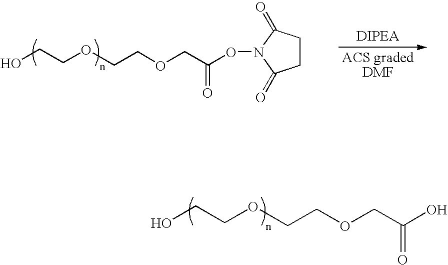
Structures of the Reagents 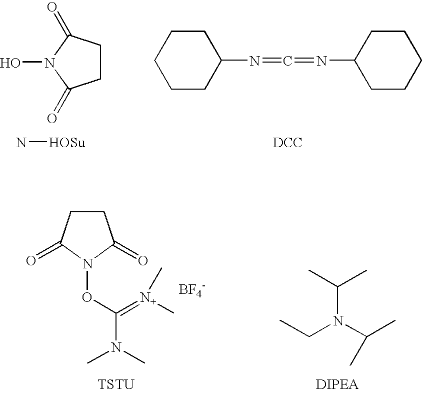
Proposed Reaction Mechanism: 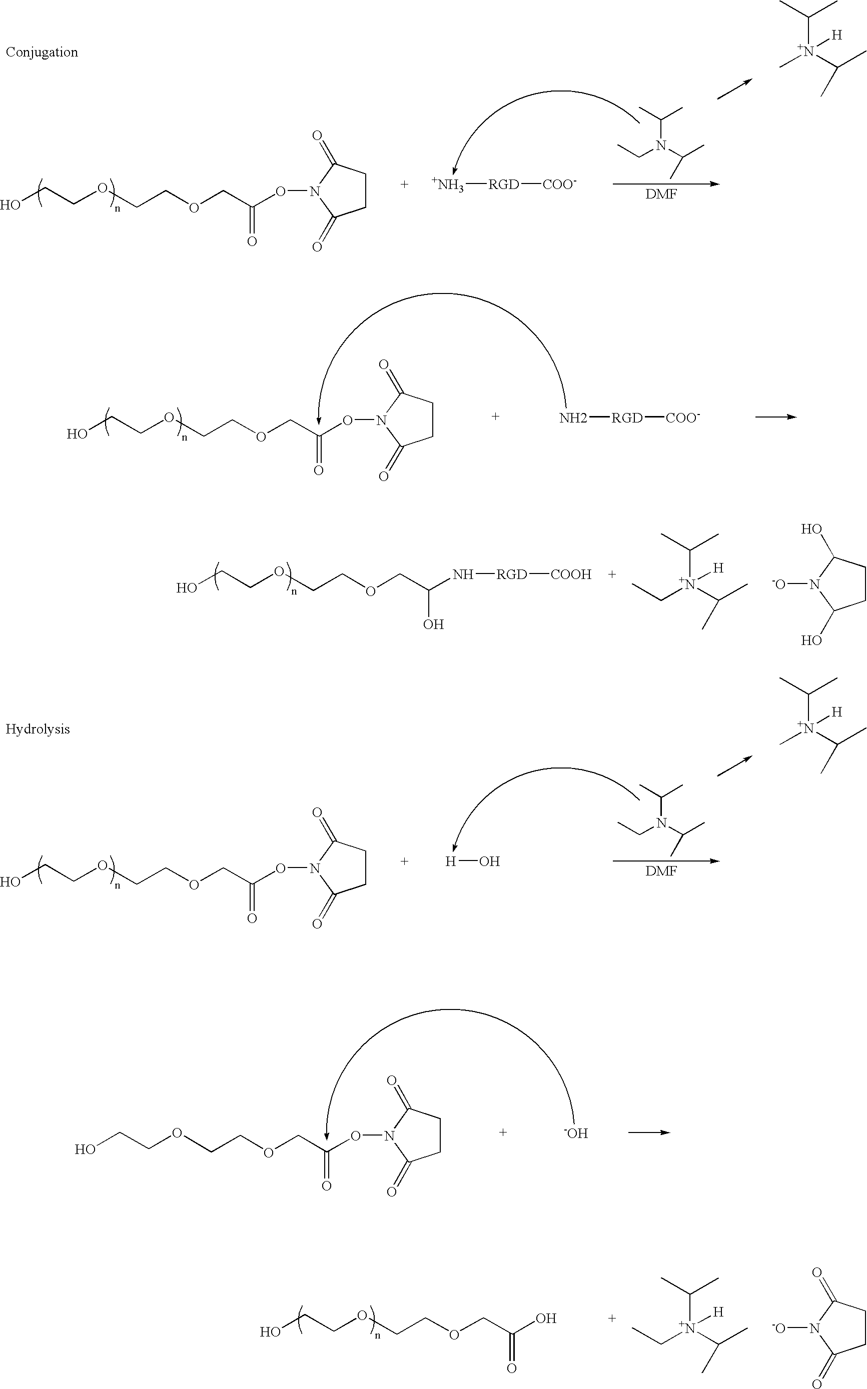
Solution for the Hydrolysis Problem: 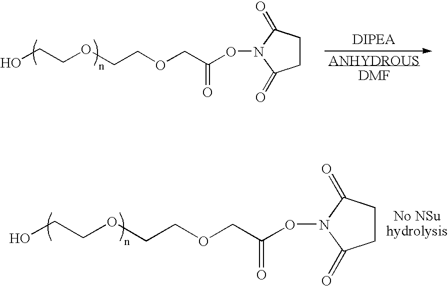
Optimized Conjugation Condition: 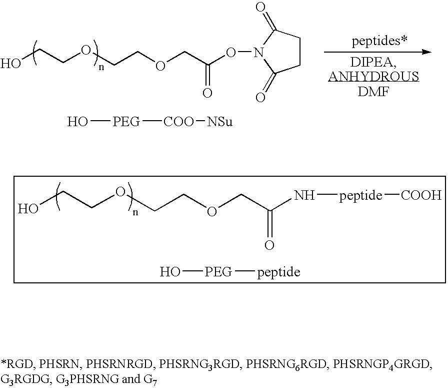
4.3.8 Acrylation of HO-PEG-NSu:

As noted earlier, another route to create substrates with surface bound peptide was to synthesize Ac-PEG-peptide first and then construct into networks directly without further treatment in solid phase. To do so, synthesizing Ac-PEG-NSu was the first step. The elution time for HO-PEG-NSu was around 11.6 min. The conversion of HO-PEG-NSu to a new compound was demonstrated by the appearance of a new peak eluted around 12.9 min. Because Ac-PEG-NSu was more hydrophobic than HO-PEG-NSu, longer elution time indicated the formation of Ac-PEG-NSu. Formation of Ac-PEG-NSu was also verified by the ¹H and ¹³C NMR (data not shown here). Converting HO-PEG-COOH to HO-PEG-NSu and to Ac-PEG-NSu all took place in THF. To simplify the procedure of synthesizing Ac-PEG-NSu directly from HO-PEG-COOH, HO-PEG-NSu was not precipitated out from THF before acrylation. The molar ratio was 1:2.5:2.5 for HO-PEG-COOH:N-HOSu:DCC during NSu activation and the molar ratio for the acrylation step was 1:3:3.5 for HO-PEG-COOH:AC:TEA. The conversion was greater than 98% with a yield of greater than 90%.

Synthesis of Ac-PEG-RGDW and Ac-PEG-RGD:

The reaction proceeded in DMF and completed after 1 h demonstrated by the disappearance of the peak at around 13.1 min for Ac-PEG-NSu and the appearance of a new peak at around 8.8 min. The new peak was associated with UV absorbance at 280 nm for Ac-PEG-RGDW and UV absorbance at 200 nm for Ac-PEG-RGD. After precipitation in cold ether and filtration, solids resulted for both reactions. However, the solids did not dissolve in acetonitrile completely. The solids were used to construct TMPTA networks but did not dissolve in TMPTA at 80° C.

Conjugation of Ac-PEG-NSu with RGD, Trp or PHSRN in DMF and Under Periodic Sonication:

During the reaction with RGDW and RGD discussed earlier, aggregates with diameters of 0.1˜0.5 mm formed in both solutions. The presence of a hydrophobic group (Ac) and a hydrophilic group (RGD or RGDW) could have facilitated the self-assembly of the molecules. To resolve the issue, sonication was introduced into the reaction system. Reaction solution was clear without any macroscopic aggregates. Applying sonication prevented the formation of macroscopic aggregates. The result also confirmed the hypothesis that aggregates formed were acrylated PEG-peptide conjugates brought together by electrostatic forces.

For the synthesis of Ac-PEG- RGD, the molar ratio of Ac-PEG-NSu:RGD:DIPEA was 1:2:2. The conversion ratio reached the highest at 77.7% after 3.5 h and decreased to 75.3% after 17 h reaction. After the product was dried, the conversion increased to 81.5%. A total of 143.6 mg product was obtained out from 220.9 mg, a yield of around 59%. However, after 17 h of reaction, the peak denoting Ac-PEG-RGD became broader. This may be due to the presence of a guanidinium group at the side chain of Arg that could compete with the N-terminal of the peptide and conjugate to the activated COOH terminal after long period of reaction time.

For the synthesis of Ac-PEG-RGDW, the molar ratio of Ac-PEG-NSu:RGDW:DIPEA was 1:1.5:1.5. The conversion peaked at 17 h with a conversion ratio of 74.1%. After the product was dried, the conversion increased to 88.6%. However, the solid product was absorbed by the filter paper and could not be collected.

For the synthesis of Ac-PEG-PHSRN, the molar ratio of Ac-PEG-NSu : PHSRN: DIPEA was 1:1.5:1.5. The conversion was close to 100% after 40 min and was stable after 17 h. After being dried, 243.5 mg product was obtained out from 220.9 mg starting material, a yield of around 90%.

For the synthesis of acrylated PEG peptide conjugates, the conversion percentage, the reaction time and the yield all varied with peptide identities.

Dried Ac-PEG-RGD and Ac-PEG-PHSRN were used to make TMPTA networks. However, the two materials did not dissolve in TMPTA at 80° C. Instead, lumps of gel-like materials formed at the bottom of the vials. Sonication might help to break down the self-association or aggregation of the two materials, but to maintain a constant temperature at 80° C. during sonication was challenging. A regulator might be used to maintain the temperature. However, the sonicator and regulator will certainly introduce more complexity into the film making procedure. The route of synthesizing Ac-PEG-peptide in the construction of TMPTA networks was excluded due to the solubility problem of PEG-peptide conjugates in TMPTA. The Ac-PEG-peptide molecules can be explored in other applications.

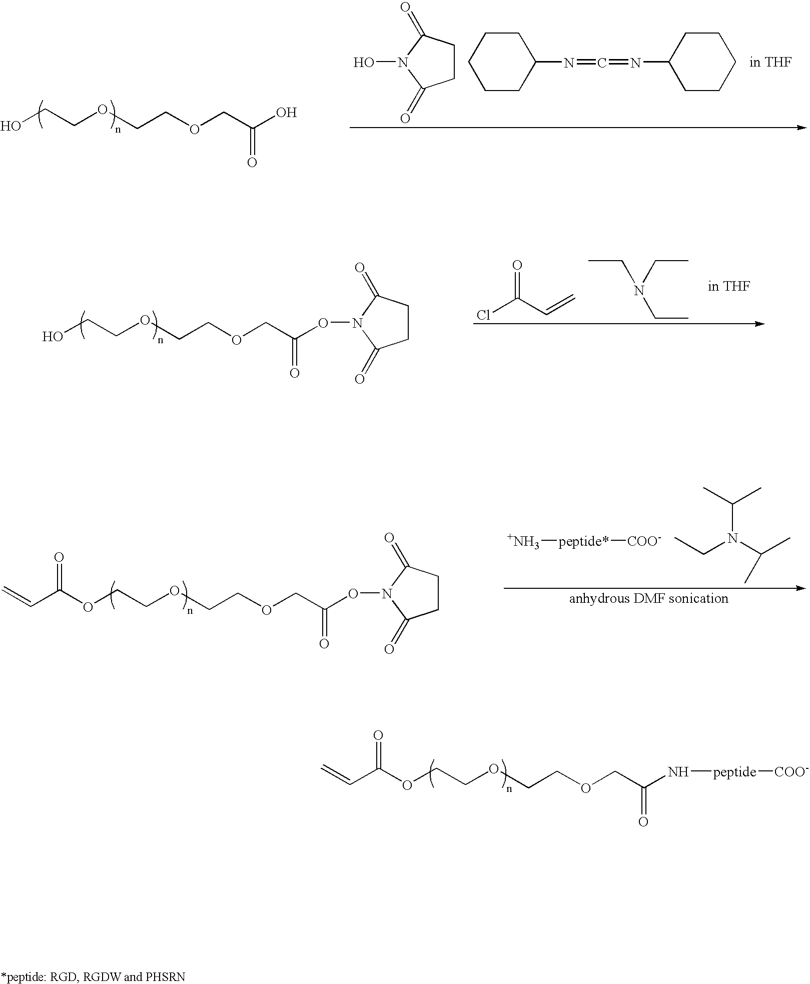

### Synthesis of Acrylated PEG-Peptide Conjugates

Two major synthesis schemes were established to make PEG-peptide conjugates. One was to synthesize HO-PEG-peptide. HO-PEG-COOH was first converted to HO-PEG-NSu by N-HOSu in dry THF with DCC as the coupling reagent. HO-PEG-NSu was then conjugated with the series of peptides (i.e., K, RGD, PHSRN, PHSRNRGD, PHSRNG₃RGD, PHSRNG₆RGD, PHSRNGP₄GRGD, G₃RGDG or G₃PHSRNG) with DIPEA as the catalysis in anhydrous DMF. The other scheme was to synthesize Ac-PEG-peptide. HO-PEG-COOH was converted to HO-PEG-NSu by N-HOSu in dry THF with DCC as the coupling reagent. In the same solution, AC and TEA were added to convert HO-PEG-NSu to Ac-PEG-NSu. Conjugation of Ac-PEG-NSu with peptides proceeded in anhydrous DMF with periodical sonication. The solution phase HO-PEG-COOH activation and peptide conjugation provided a facile way to optimize conditions for activate and graft peptides to surface COOH groups on solid substrates.

### REFERENCES

1. Y. Inada, M. Furukawa, H. Sasaki, Y. Kodera, M. Hiroto, H. Nishimura, and A. Matsushima, Trends Biotechnol. 13:86 (1995).
2. C. Delgado, G. E. Francis, and D. Fisher, Crit. Rev. Ther. Drug Carrier Syst. 9:249 (1992).
3. R. Mehvar, J. Pharm. Pharm. Sci. 3:125 (2000).
4. G. Fortier, Biotechnol. Genet. Eng. Rev. 12:329 (1994).
5. J. M. Harris and S. Zalipsky, “Poly(ethylene glycol) Chemistry and Biological Applications,” American Chemical Society, Washington, D.C. (1997).
6. S. Zalipsky and G. Barany, J. Bioact. Biocompatible Polym. 5:227 (1990).
7. M. Yokoyama et al., Bioconjugate Chem. 3:275 (1992).
8. T. Nakamura, Y. Nagasaki, et al., Bioconjugate Chem. 9:300 (1998).
9. Y. Nagasaki et al., Macromolecules, 30:6489 (1997).
10. P. D. Drumheller and J. A. Hubbell, J. Biomed. Mater. Res. 29:207 (1995).
11. W. J. Kao and J. A. Hubbell J A, Biotech. Bioengrn., 59:2 (1998).
12. W. J. Kao, D. Lee, J. C. Schense, and J. A. Hubbell, J. Biomed. Mater. Res., 55(1), 79-88 (2001).
13. W. J. Kao and D. Lee, Biomaterials, 22(21), 2901-9 (2001). 14. R. D. Brown, R. Champion, P. S. Elmes, and P. D. Godfrey. J. Am. Chem. Soc., 107:4109 (1985).
15. The Chemistry of Acrylonitrile. 2d ed. The American Cyanamid Company, New York, N.Y., 17 (1959).
16. H. A. Bruson. Organic Reactions, 5:79 (1949).
17. H. Houben-Weyl, E. Muller, und T. Verlag. “Methoden der Organischen Chemie.” Stuttgart, XIII, 377 (1970).
18. A. F. Buckmann and M. Morr. Makromol. Chem., 182:1379 (1981).
19. J. M. Harris, J. M. Dust, M. R. Sedaghat-Herati et. al., Am. Chem. Soc., Polymer Preprints, 30:356 (1989).
20. J. M. Harris. Macromol. Chem. Phys., C25:325 (1985).

1. A hydrogel comprising:
a first polymer matrix; and
a bifunctional modifier comprising a poly(alkylene glycol) molecule having a substituted or unsubstituted α-terminus and a substituted or unsubstituted ω-terminus, and wherein at least one of the α- or ω-termini is covalently bonded to the first polymer matrix.

2. The hydrogel of claim 1, further comprising a pharmacologically-active agent covalently bonded to one of the α- or ω-termini that is not bonded to the first polymer matrix.

3. The hydrogel of claim 1, wherein the first polymer matrix is proteinaceous.

4. The hydrogel of claim 1, wherein the first polymer matrix contains an amino group and wherein at least one of the α- or ω-termini is covalently bonded to the amino group.

5. The hydrogel of claim 1, wherein the first polymer matrix is selected from the group consisting of gelatin, calcium alginate, calcium/sodium alginate, collagen, oxidized regenerated cellulose, carboxymethylcellulose, amino-modified cellulose, and whey protein.

6. The hydrogel of claim 1, wherein the first polymer matrix is selected from the group consisting of gelatin and collagen.

7. The hydrogel of claim 1, wherein the first polymer matrix is cross-linked with a cross-linking reagent.

8. The hydrogel of claim 1, wherein the first polymer matrix is cross-linked with glutaraldehyde.

9. The hydrogel of claim 1, wherein the first polymer matrix further comprises EDTAD moieties bonded to it.

10. The hydrogel of claim 1, wherein the α-terminus and the ω-terminus of the poly(alkylene glycol) molecule are different from one another.

11. The hydrogel of claim 1, wherein the α-terminus and the ω-terminus are substituted with a moiety selected from the group consisting of halo, hydroxy, C₁-C₂₄-alkyl, C₁-C₂₄-alkenyl, C₁-C₂₄-alkynyl, C₁-C₂₄-alkoxy, C₁-C₂₄-heteroalkyl, C₁-C₂₄-heteroalkenyl, C₁-C₂₄-heteroalkynyl, cyano-C₁-C₂₄-alkyl, C₃-C₁₀-cycloalkyl, C₃-C₁₀-cycloalkenyl, C₃-C₁₀-cycloalkynyl, C₃-C₁₀-cycloheteroalkyl, C₃-C₁₀-cycloheteroalkenyl, C₃-C₁₀-cycloheteroalkynyl, acyl, acyl-C₁-C₂₄-alkyl, acyl-C₁-C₂₄-alkenyl, acyl-C₁-C₂₄-alkynyl, carboxy, C₁-C₂₄-alkylcarboxy, C₁-C₂₄-alkenylcarboxy, C₁-C₂₄-alkynylcarboxy, carboxy-C₁-C₂₄-alkyl, carboxy-C₁-C₂₄-alkenyl, carboxy-C₁-C₂₄-alkynyl, aryl, aryl-C₁-C₂₄-alkyl, aryl-C₁-C₂₄-alkenyl, aryl-C₁-C₂₄-alkynyl, heteroaryl, heteroaryl-C₁-C₂₄-alkyl, heteroaryl-C₁-C₂₄-alkenyl, heteroaryl-C₁-C₂₄-alkynyl, sulfonate, arylsulfonate, and heteroarylsulfonate.

12. The hydrogel of claim 11, wherein the moiety on the α-terminus is different from the moiety on the ω-terminus.

13. The hydrogel of claim 11, wherein the moieties on the α-terminus and the ω-terminus are substituted or unsubstituted, and when substituted bear a substituent selected from the group consisting of alkyl, aryl, acyl, halogen, hydroxy, amino, alkoxy, alkylamino, acylamino, thioamido, acyloxy, aryloxy, aryloxyalkyl, mercapto, thia, aza, oxo, saturated cyclic hydrocarbon, unsaturated cyclic hydrocarbon, heterocycle, aryl, and heteroaryl.

14. The hydrogel of claim 1, further comprising a pharmacologically-active agent entrained within the hydrogel.

15. The hydrogel of claim 1, further comprising living cells entrained within the hydrogel.

16. The hydrogel of claim 1, further comprising a second polymer matrix, wherein the second polymer matrix interpenetrates with the first polymer matrix.

17. The hydrogel of claim 16, wherein the second polymer matrix comprises a photopolymerized poly(acrylate).

18. The hydrogel of claim 16, wherein the second polymer matrix comprises one or more monomers selected from the group consisting of α-acrylate-ω-acrylate-poly(alkylene glycol), trimethylolpropane triacrylate, acrylic acid, and acryloyl halide.

19. The hydrogel of claim 16, further comprising a pharmacologically-active agent covalently bonded to one of the α- or ω-termini that is not bonded to the first polymer matrix.

20. The hydrogel of claim 16, further comprising a pharmacologically-active agent entrained within the hydrogel.

21. The hydrogel of claim 16, further comprising living cells entrained within the hydrogel.

22. A hydrogel comprising:
a first polymer matrix containing reactive amino acid moieties;
a second polymer matrix, wherein the second polymer matrix interpenetrates with the first polymer matrix; and
a bifunctional modifier comprising a compound of formula: 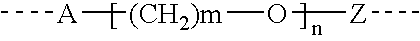
wherein at least one of the “A” or “Z” moieties is covalently bonded to the reactive amino moieties of the polymer matrix; and wherein “A” and “Z” are independently selected from the group consisting of hydrogen, halo, hydroxy, C₁-C₂₄-alkyl, C₁-C₂₄-alkenyl, C₁-C₂₄-alkynyl, C₁-C₂₄-alkoxy, C₁-C₂₄-heteroalkyl, C₁-C₂₄-heteroalkenyl, C₁-C₂₄-heteroalkynyl, cyano-C₁-C₂₄-alkyl, C₃-C₁₀-cycloalkyl, C₃-C₁₀-cycloalkenyl, C₃-C₁₀-cycloalkynyl, C₃-C₁₀-cycloheteroalkyl, C₃-C₁₀-cycloheteroalkenyl, C₃-C₁₀-cycloheteroalkynyl, acyl, acyl-C₁-C₂₄-alkyl, acyl-C₁-C₂₄-alkenyl, acyl-C₁-C₂₄-alkynyl, carboxy, C₁-C₂₄-alkylcarboxy, C₁-C₂₄-alkenylcarboxy, C₁-C₂₄-alkynylcarboxy, carboxy-C₁-C₂₄-alkyl, carboxy-C₁-C₂₄-alkenyl, carboxy-C₁-C₂₄-alkynyl, aryl, aryl-C₁-C₂₄-alkyl, aryl-C₁-C₂₄-alkenyl, aryl-C₁-C₂₄-alkynyl, heteroaryl, heteroaryl-C₁-C₂₄-alkyl, heteroaryl-C₁-C₂₄-alkenyl, heteroaryl-C₁-C₂₄-alkynyl, sulfonate, arylsulfonate, and heteroarylsulfonate;
“m” is an integer of from 2 to 8
“n” is an integer equal to or greater than 100.

23. The hydrogel of claim 22, wherein the second polymer matrix comprises a photopolymerized poly(acrylate).

24. The hydrogel of claim 22, wherein the second polymer matrix comprises one or more monomers selected from the group consisting of α-acrylate-ω-acrylate-poly(alkylene glycol), trimethylolpropane triacrylate, and acrylic acid.

25. The hydrogel of claim 22, further comprising a pharmacologically-active agent covalently bonded to one of the “A” or “Z” moieties that is not bonded to the first polymer matrix.

26. The hydrogel of claim 22, further comprising a pharmacologically-active agent entrained within the hydrogel.

27. The hydrogel of claim 22, further comprising living cells entrained within the hydrogel.

28. A hydrogel comprising:
a first polymer matrix;
a bifunctional modifier comprising a poly(alkylene glycol) molecule having a substituted or unsubstituted α-terminus and a substituted or unsubstituted ω-terminus, and wherein at least one of the α- or ω-termini is covalently bonded to the first polymer matrix; and
a second polymer matrix, wherein the second polymer matrix interpenetrates with the first polymer matrix.

29. The hydrogel of claim 28, wherein the first polymer matrix is proteinaceous and the second polymer matrix comprises a photopolymerized poly(acrylate).

30. The hydrogel of claim 28, wherein the first polymer matrix is selected from the group consisting of gelatin and collagen, and the second polymer matrix comprises a photopolymerized poly(acrylate).

31. The hydrogel of claim 28, further comprising a pharmacologically-active agent covalently bonded to one of the α- or ω-termini that is not bonded to the first polymer matrix.

32. The hydrogel of claim 28, further comprising a pharmacologically-active agent entrained within the hydrogel.

33. The hydrogel of claim 28, further comprising living cells entrained within the hydrogel.

34. A method of making a hydrogel comprising:
reacting a first polymer matrix with a bifunctional modifier comprising a poly(alkylene glycol) molecule having a substituted or unsubstituted α-terminus and a substituted or unsubstituted ω-terminus, whereby at least one of the α- or ω-termini is covalently bonded to the polymer matrix.

35. The method of claim 34, further comprising cross-linking the first polymer matrix with a cross-linking reagent.

36. The method of claim 35, wherein the first polymer matrix is cross-linked with glutaraldehyde.

37. The method of claim 34, further comprising reacting EDTAD with the first polymer matrix for a time and under conditions wherein the EDTAD binds to the polymer matrix.

38. The method of claim 34, further comprising reacting the bifunctional modifier with a pharmacologically-active agent, whereby the pharmacologically-active agent is covalently bonded to one of the the α- or ω-termini that is not bonded to the first polymer matrix.

39. The method of claim 34, wherein the c-terminus and the o)-terminus of the bifunctional modifier are different from one another.

40. The method of claim 34, wherein the first polymer matrix is selected from the group consisting of gelatin, calcium alginate, calcium/sodium alginate, collagen, oxidized regenerated cellulose, carboxymethylcellulose, amino-modified cellulose, and whey protein.

41. The method of claim 34, wherein the first polymer matrix is selected from the group consisting of gelatin and collagen.

42. The method of claim 34, further comprising entraining a pharmacologically-active agent within the hydrogel.

43. The method of claim 34, further comprising contacting the first polymer matrix with a plurality of monomers and then polymerizing the monomers to yield a second polymer matrix, wherein the second polymer matrix interpenetrates with the first polymer matrix.

44. The method of claim 43, wherein the plurality of monomers comprises photopolymerized poly(acrylates) and the monomers are polymerized by exposure to infrared, visible, or ultraviolet radiation.

45. The method of claim 43, wherein the plurality of monomers comprises one or more monomers selected from the group consisting of α-acrylate-ω-acrylate-poly(alkylene glycol), trimethylolpropane triacrylate, acrylic acid, and acryloyl halide.

46. The method of claim 43, further comprising covalently bonding a pharmacologically-active agent to one of the α- or ω-termini that is not bonded to the first polymer matrix.

47. The method of claim 43, further comprising entraining a pharmacologically-active agent within the hydrogel.

48. The method of claim 43, further comprising entraining living cells within the hydrogel.

49. A method of administering pharmacologically-active agents or cells to a patient in need thereof, the method comprising entraining pharmacologically-active agents or cells within a hydrogel as recited in claim 1, and then administering the hydrogel to a patient in need of the pharmacologically-active agent or cells.

50. A method of administering pharmacologically-active agents or cells to a patient in need thereof, the method comprising entraining pharmacologically-active agents or cells within a hydrogel as recited in claim 22, and then administering the hydrogel to a patient in need of the pharmacologically-active agent or cells.

51. A method of administering pharmacologically-active agents or cells to a patient in need thereof, the method comprising entraining pharmacologically-active agents or cells within a hydrogel as recited in claim 28, and then administering the hydrogel to a patient in need of the pharmacologically-active agent or cells.
